(19) United States
(12) Patent Application Publication
(10) Pub. No.: US 2015/0275307 A1
(43) Pub. Date: Oct. 1, 2015

(54) COMPOSITIONS AND METHODS FOR DETECTING SESSILE SERRATED ADENOMAS/POLYPS

(71) Applicant: UNIVERSITY OF UTAH RESEARCH FOUNDATION, Salt Lake City,, UT (US)

(72) Inventors: Curt Hagedorn, Salt Lake City, UT (US); Don Delker, Farmington, UT (US); Randall Burt, Sandy, UT (US)

(21) Appl. No.: 14/436,100

(22) PCT Filed: Oct. 16, 2013
(86) PCT No.: PCT/US13/65305

§ 371 Date: Apr. 16, 2015

Related U.S. Application Data
(60) Provisional application No. 61/714,482, filed on Oct. 16, 2012.
(60) Provisional application No. 61/780,930, filed on Mar. 13, 2013.

(51) Int. Cl. C12Q 1/68; G01N 33/50

(52) U.S. Cl. CPC C12Q 1/6886; G01N 33/5091; C12Q 2600/16; C12Q 2600/158; C12Q 2600/156; G01N 2800/60; G01N 2800/06; C12Q 2600/112

(57) ABSTRACT

Provided are methods of predicting the likelihood that a colorectal polyp in a subject will develop into colorectal cancer. Further provided are methods of increasing the likelihood of detecting colorectal cancer at an early stage, the methods including predicting the likelihood that a colorectal polyp in a subject will develop into colorectal cancer, and when there is an increased likelihood that the colorectal polyp will develop into colorectal cancer, the frequency of colonoscopies administered to the subject are increased. Further provided are kits for predicting the likelihood that a colorectal polyp in a subject will develop into colorectal cancer.

### CROSS-REFERENCE TO RELATED APPLICATIONS

This application claims priority to U.S. Provisional Patent Application No. 61/714,482, filed Oct. 16, 2012, and U.S. Provisional Patent Application No. 61/780,930, filed Mar. 13, 2013, each of which is incorporated herein by reference in its entirety.

### STATEMENT REGARDING FEDERALLY SPONSORED RESEARCH

This invention was made with government support under grants CA148068, CA073992, and CA146329 awarded by the National Institutes of Health. The government has certain rights in the invention.

### FIELD

This disclosure relates to compositions and methods for detecting and diagnosing sessile serrated polyps and determining risk of progression to colorectal cancer.

### INTRODUCTION

Colon cancer remains the second leading cause of death among cancer patients in the United States. Each year more than 100,000 new cases of colon cancer are diagnosed and more than 50,000 deaths occur due to colon cancer. Current preventative strategies include screening colonoscopies every 10 years in men and women over 50 years of age and more frequently in individuals with first degree relatives with colon cancer. The presence of large and/or many polyps throughout the colon are suggestive of an increased risk for cancer since many polyps may progress to malignant adenocarcinoma. Although much is known regarding the progression of classic adenomatous polyps to colon cancer, less is known regarding the progression of serrated polyps to colon cancer. Serrated polyps are also frequently found during routine colonoscopies but due to their often small size and lack of dysplastic features have been frequently overlooked as benign lesions. Recent studies suggest that large, right-sided, sessile serrated adenomas/polyps (SSA/Ps) have a significant risk of developing into adenocarcinoma, and that such polyps probably account for 20-30% of colon cancers. SSA/Ps are characterized by their exaggerated serration, horizontally extended crypts, nuclear atypia, and a mucus cap that often makes endoscopic detection difficult. Small SSA/Ps can increase in size and the exact relationship between size of SSA/Ps and risk for colon cancer remains to be defined. However, it is frequently difficult to distinguish, both endoscopically and histologically, small SSA/Ps from hyperplastic polyps that are considered to have no significant risk for progression to colon cancer.

The term “serrated adenoma” was first suggested as colorectal polyps that exhibited the architectural but not the cytologic features of a hyperplastic polyp. The early evidence of “hyperplastic polyposis” was presented when “multiple metaplastic polyps” were noted in patients that had multiple colon polyps exhibiting features of hyperplastic polyps. Later, “serrated adenomatous polyposis” were described in patients with morphological features of serrated polyps and some also having evidence of adenocarcinoma. Serrated polyp pathway has been described that suggests an alternative route of colon cancer development in patients with serrated polyps. Hyperplastic polyposis or serrated polyposis syndrome is an extreme phenotype with occurrence of multiple serrated polyps and a high risk for colon cancer.

The term “hyperplastic polyposis” was changed to “serrated polyposis” by the World Health Organization (WHO) classification due to occurrence of sessile serrated adenoma/polyps (SSA/P) in this syndrome. As per the classification, “serrated polyposis” is defined as patients with (a) at least five serrated polyps proximal to the sigmoid colon with two or more of these being more than 10 mm; (b) any number of serrated polyps proximal to the sigmoid colon in an individual who has a first-degree relative with serrated polyposis; or (c) more than 20 serrated polyps of any size, but distributed throughout the colon.

Serrated polyposis syndrome (SPS) has been shown to have higher risk of colorectal cancer. Prior large cohorts (n>40) of SPS patients have shown 7% to 42% increased risk of colorectal cancer development. Some smaller cohorts have shown CRC risk up to 77%. Family history and high risk of CRC in relatives of SPS has been documented, suggesting a genetic predisposition. However, a genetic basis for serrated polyposis syndrome has not been found.

### SUMMARY

In some aspects, provided are methods of predicting the likelihood that a colorectal polyp in a subject will develop into colorectal cancer. The methods may include determining an expression level of at least one gene selected from MUC17, VSIG1, and CTSE in a sample obtained from the colorectal polyp; comparing the expression level to a control value associated with that same gene; and predicting the likelihood that the colorectal polyp will develop into colorectal cancer based on the relative difference between the expression level and the control value associated with each gene, wherein an increase in the expression level at least one of MUC17, VSIG1, and CTSE relative to the control value associated with each gene correlates with an increased likelihood of the colorectal polyp developing into colorectal cancer. In some embodiments, the methods further include determining an expression level of TFF2 in the sample obtained from the colorectal polyp, wherein an increase in the expression level of TFF2 relative to the control value associated with TFF2 correlates with an increased likelihood of the colorectal polyp developing into colorectal cancer. In some embodiments, the methods further include determining an expression level of at least one gene selected from TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, in a sample obtained from the colorectal polyp, wherein an increase in the expression level at least one of TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, and ONECUT2 relative to the control value associated with each gene correlates with an increased likelihood of the colorectal polyp developing into colorectal cancer, and wherein a decrease in the expression level at least one of SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1 relative to the control value associated with each gene correlates with an increased likelihood of the colorectal polyp developing into colorectal cancer. In some embodiments, the methods further include determining the expression level of at least one gene selected from MUC5AC, KLK10, TFF1, DUOX2, CDH3, S100P, and GJB5 in the sample obtained from the colorectal polyp, wherein an increase in the expression level of at least one of MUC5AC, KLK10, TFF1, DUOX2, CDH3, S100P, and GJB5 relative to the control value associated with the gene correlates with an increased likelihood of the colorectal polyp developing into colorectal cancer. In some embodiments, the methods further include determining the expression level of at least one gene selected from SLC14A2, CD177, ZG16, and AQP8 in the sample obtained from the colorectal polyp, wherein a decrease in the expression level of at least one of SLC14A2, CD177, ZG16, and AQP8 relative to the control value associated with the gene correlates with an increased likelihood of the colorectal polyp developing into colorectal cancer.

In some embodiments, when the expression level of at least one of MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, MUC5AC, KLK10, TFF1, DUOX2, CDH3, S100P, and GJB5 is greater than the control value, the method further includes diagnosing the polyp as being a sessile serrated adenoma/polyp. In some embodiments, when the control value is greater than the expression level of at least one of SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, TMIGD1, SLC14A2, CD177, ZG16, and AQP8, the method further includes diagnosing the polyp as being a sessile serrated adenoma/polyp. In some embodiments, the methods further include diagnosing the subject as having serrated polyposis syndrome.

In some embodiments, the control value associated with each gene is determined by determining the expression level of that gene in one or more control samples, and calculating an average expression level of that gene in the one or more control samples, wherein each control sample is obtained from healthy colonic tissue of the same or a different subject. In some embodiments, determining the expression level of at least one gene comprises measuring the expression level of an RNA transcript of the at least one gene, or an expression product thereof.

In some embodiments, measuring the expression level of the RNA transcript of the at least one gene, or the expression product thereof, includes using at least one of a PCR-based method, a Northern blot method, a microarray method, and an immunohistochemical method. In some embodiments, the methods include determining the expression level of at least three genes.

In other aspects, provided are methods of determining the frequency of colonoscopies for a subject. The methods may include predicting the likelihood that a colorectal polyp in a subject will develop into colorectal cancer according to the methods detailed herein, wherein when there is an increased likelihood that the colorectal polyp will develop into colorectal cancer, increasing the frequency of colonoscopies administered to the subject.

In other aspects, provided are methods of increasing the likelihood of detecting colorectal cancer at an early stage. The methods may include predicting the likelihood that a colorectal polyp in a subject will develop into colorectal cancer according to the methods detailed herein, wherein when there is an increased likelihood that the colorectal polyp will develop into colorectal cancer, increasing the frequency of colonoscopies administered to the subject.

In other aspects, provided are kits for predicting the likelihood that a colorectal polyp in a subject will develop into colorectal cancer. The kit may include at least one primer, each adapted to amplify an RNA transcript of one gene independently selected from TM4SF4, VSIG1, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, and instructions for use. In some embodiments, the kits further include at least one additional primer, each adapted to amplify an RNA transcript of one gene independently selected from MUC5AC, KLK10, CTSE, TFF2, MUC17, TFF1, DUOX2, CDH3, S100P, GJB5, SLC14A2, CD177, ZG16, and AQP8.

In other aspects, provided are kits for predicting the likelihood that a colorectal polyp in a subject will develop into colorectal cancer. The kit may include one or more probes, each adapted to specifically bind to an RNA transcript, or an expression product thereof, of one gene independently selected from TM4SF4, VSIG1, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, and instructions for use. In some embodiments, the kits further include one or more additional probes, each adapted to specifically bind to an RNA transcript, or an expression product thereof, of one gene independently selected from MUC5AC, KLK10, CTSE, TFF2, MUC17, TFF1, DUOX2, CDH3, S100P, GJB5, SLC14A2, CD177, ZG16, and AQP8. In some embodiments, at least one probe comprises an antibody to an expression product. In some embodiments, at least one probe comprises an oligonucleotide complementary to an RNA transcript.

The disclosure provides for other aspects and embodiments that will be apparent in light of the following detailed description and accompanying Figures.

### BRIEF DESCRIPTION OF THE DRAWINGS

FIG. 1. Endoscopic phenotype of four representative sessile serrated polyps/adenomas (SSA/Ps) located in the ascending colon of patients with the serrated polyposis syndrome. Panel A. Large 15 mm diameter SSA/P with a mucus cap. Panel B. 20 mm diameter SSA/P. Panel C. 10 mm diameter SSA/P. Panel D. Small 4 mm diameter SSA/P. The size of polyps was estimated using biopsy forceps as a reference. Histopathology analyses were consistent with SSA/Ps.

FIG. 2. Differentially expressed genes in sessile serrated adenoma/polyps (SSA/Ps) by RNA sequencing (RNA-seq) and microarray analyses. Panel A. RNA-seq analysis identified 1294 genes (875 increased, 419 decreased) that were significantly differentially expressed (fold change ≧1.5, FDR<0.05) in SSA/Ps as compared to control colon biopsies. Differentially expressed genes in SSA/Ps that were found by RNA-seq analysis (red) and those found in a microarray study (green; 101 total, 59 increased, 42 decreased) are shown in the Venn diagram (23). Panel B. Hierarchical clustering of the differentially expressed genes in Panel A. Note: only 782 genes could be compared in the hierarchical clustering analysis because fewer genes were interrogated in the microarray analysis. Panel C. Hierarchical clustering of differentially expressed genes in SSA/Ps identified by RNA-seq analysis and in adenomatous polyps (APs) identified by microarray analysis (24). 136 genes (75 increased, 61 decreased) with a fold change ≧10 and FDR of <0.05 from both datasets were compared. Four distinct clusters are shown, cluster 1 represents genes increased in only SSA/Ps, cluster 2 represents genes increased in both SSA/Ps and APs, cluster 3 represents genes decreased only in APs, and cluster 4 represents genes decreased in both SSA/Ps and APs. Note: the full range of fold change is not reflected in color bar scale, the maximum fold change in RNA-seq analysis was 582-fold (MUC5AC) in SSA/Ps and 208-fold (GCG) in APs by microarray analysis.

FIG. 3. Expression of mucin 17 (MUC17), V-set and immunoglobulin domain containing 1 (VSIG1), gap junction protein, beta 5 (GJB5) and regenerating islet-derived family member 4 (REG4) in SSA/Ps, adenomatous polyps (APs) and controls as measured by RNA-seq analysis. Panel A1. MUC17 RNA-seq results. The y-axis represents the number of uniquely mapped sequencing reads per kilobase of transcript length per million total reads (RPKM) mapped to the MUC17 locus. The x-axis represents the chromosome (Chr) 7 coordinates and gene structure of the MUC17 transcript. Analysis showed an 82-fold increase in MUC17 mRNA in SSA/Ps (red, n=7 polyps) compared to uninvolved colon (patient matched uninvolved, blue, n=6) and control colon (screening colon without polyps; green, n=2). The sequencing read length was 50 base pairs. Panel A2. MUC17 expression measured by qPCR analysis in SSA/Ps, adenomatous polyps and controls in additional patients. Relative mRNA levels of MUC17 in large (>1 cm) and small (<1 cm) SSA/Ps (n=21), adenomatous polyps (n=10), uninvolved colon and normal control colon biopsies (n=10 each) are shown. In small and large SSA/Ps, MUC17 expression was increased by 38 and 71-fold, respectively, compared to controls. qPCR results were normalized to β-actin. The average MUC17 expression level in uninvolved colon tissue was chosen as the baseline. P-values were calculated using the Mann-Whitney U-test. Panel B1. VSIG1 (Chr X) RNA-seq results. A 106-fold increase in expression of VSIG1 was found in SSA/Ps as compared to controls. Panel B2. VSIG1 qPCR results. In small and large SSA/Ps, VSIG1 expression was increased 969 and 1393-fold, respectively. Panel C1. GJB5 (Chr 1) RNA-seq results. A 27-fold increase in GJB5 mRNA was found in SSA/Ps. Panel C2. GJB5 qPCR results. In small and large SSA/Ps, GJB5 expression was increased 446 and 523-fold, respectively. Panel D1. REG4 (Chr 1) RNA-seq results. An 87-fold increase in REG4 mRNA was found in SSA/Ps. Panel D2. REG4 qPCR results. In small and large SSA/Ps, REG4 mRNA was increased 68 and 116-fold, respectively.

FIG. 4. Immunostaining for VSIG1, MUC17, CTSE and TFF2 in control colon, SSA/Ps, hyperplastic and adenomatous polyps. Representative images of immunoperoxidase staining with affinity purified polyclonal antibodies and formalin-fixed, paraffin-embedded biopsies of patient matched and normal control colon (Panel A, n≧15, see Methods), syndromic SSA/Ps (Panel B, n≧10), sporadic SSA/Ps (Panel C, n≧15), hyperplastic polyps (Panel D, n≧10) and adenomatous polyps (Panel E, n≧10) are shown. Representative immunohistochemical stains for REG4 in control and polyp specimens are provided in FIG. 6.

FIG. 5. Expression of adolase B (ALDOB) in mRNA SSA/Ps, adenomatous polyps (Adenoma) and controls. Panel A. ALDOB RNA sequencing results. The y-axis represents RPKM. The x-axis represents the coordinates and gene structure of the ALDOB transcript. Bioinformatic analysis revealed a 20-fold increase in ALDOB mRNA in SSA/Ps (red, n=7 polyps) compared to controls (blue and green). Panel B. Relative mRNA levels of ALDOB in small and large SSA/Ps n=21), adenomatous polyps (n=10), right uninvolved colon of serrated polyposis syndrome patients (n=10) and control right colon (screening colonoscopy with no polyps; (n=10) were measured by qPCR relative to β-actin. In small and large SSA/Ps ALDOB expression was greater by 33 and 38-fold, respectively, compared to controls.

FIG. 6. Immunostaining for REG4 in control colon, SSA/Ps, hyperplastic and adenomatous polyps and higher magnification view of VSIG1 staining of an SSA/P. Representative images of immunoperoxidase staining with affinity purified polyclonal antibodies and formalin-fixed, paraffinembedded biopsies of control colon (Panel A, n≧15), syndromic SSA/Ps (Panel B, n≧9), sporadic SSA/Ps (Panel C, n≧15), hyperplastic polyps (Panel D, n≧10) and adenomatous polyps (Panel E, n≧10) are shown. Immunostaining methods are described in detail in Methods. A representative higher magnification view of VSIG1 immunostaining of an SSA/P is shown (Panel F).

FIG. 7. Table of the top 50 gene transcripts increased in sessile serrated polyps (SSA/P) in serrated polyposis patients compared to controls. Fold change is reported for seven right-sided sessile serrated polyps, from five serrated polyposis patients (age 26-62 years, 3 female and 2 male), compared to surrounding uninvolved colon and normal colon from healthy volunteers (controls, n=8). Fold-change (Fold) and false discovery rate (FDR) are provided. The fold change and FDR in sex matched adenomatous polyps (AP) (age 55-79 years, five right-sided and two left-sided) with low dysplasia compared to uninvolved colon (n=7) from a previous microarray study are provided (Sabates-Bellver, et al., 2007; PMID 18171984). Genes with an asterisk have not been previously reported to be differentially expressed in SSA/Ps. “na” denotes transcripts not analyzed in the microarray study.

FIG. 8. Table of the top 25 gene transcripts decreased in sessile serrated polyps (SSA/P) in serrated polyposis patients compared to controls. Fold change is reported for seven right-sided sessile serrated polyps (four >1 cm), from five serrated polyposis patients (age 26-62 years, three female and two male), compared to surrounding uninvolved colon and normal colon from healthy volunteers controls, (n=8). Fold-change (Fold) and false discovery rate (FDR) are shown. The fold change and FDR in sex matched adenomatous polyps (AP) (age 55-79 years, five right-sided and two left-sided) with low dysplasia compared to uninvolved colon (n=7) from a previous microarray study (Sabates-Bellver, et al., 2007; PMID 18171984). Genes with an asterisk have not been previously reported to be differentially expressed in SSA/Ps. “na” denotes transcripts not analyzed in the microarray study.

### DETAILED DESCRIPTION

The inventors have characterized the transcriptome of sessile serrated adenomas/polyps (SSA/Ps) in serrated polyposis patients. As detailed in the Examples, the transcriptome was characterized using a novel approach of RNA sequencing of 5′ capped RNAs from colon biospecimens that increases the sensitivity in identifying differentially expressed genes. Colon tissue biopsies were obtained from the ascending colon to reduce gene expression differences that may occur when comparing different segments of the colon. Colon tissue biopsies from large (more than 1 cm) right-sided SSA/Ps were also used because they are the most strongly associated with progression to colon cancer. As detailed in the Examples, differentially expressed genes in serrated polyposis patients have been discovered, including multiple genes important in colon mucosa integrity, cell adhesion, and cell development. The genes are unique to SSA/Ps and are not differentially expressed in adenomatous polyps. The gene expression results were confirmed with quantitative PCR of select RNA transcripts in additional syndromic patients. The gene expression data on syndromic SSA/Ps detailed herein reveals a panel of differentially expressed genes that are unique to SSA/Ps, may be used to improve the diagnosis of these lesions, and are novel markers for serrated polyposis. As serrated polyposis syndrome (SPS) has been shown to have higher risk of colorectal cancer, the genes disclosed herein may also be used as novel markers for determining the risk of developing colorectal cancer. The genes disclosed herein may also be used as novel markers for determining the frequency of screenings such as colonoscopies. Thus, in a broad sense, the disclosure relates to compositions and methods for detecting and diagnosing sessile serrated polyps and determining risk of progression to colorectal cancer.

In certain embodiments, provided are methods of predicting the likelihood that a colorectal polyp in a subject will develop into colorectal cancer. A subject can be an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In some embodiments, the subject is a mammal. In further embodiments, the mammal is a human.

The methods may include determining an expression level of at least one gene selected from MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, in a sample obtained from the colorectal polyp. In some embodiments, the methods include determining the expression level of at least two genes, at least three genes, or at least four genes. In some embodiments, the methods include determining the expression level of at least one of MUC17, VSIG1, and CTSE. In some embodiments, the methods further include determining the expression level of TFF2.

As used herein, the term “sample” or “biological sample” relates to any material that is taken from its native or natural state, so as to facilitate any desirable manipulation or further processing and/or modification. A sample or a biological sample can comprise a cell, a tissue, a fluid (e.g., a biological fluid), a protein (e.g., antibody, enzyme, soluble protein, insoluble protein), a polynucleotide (e.g., RNA, DNA), a membrane preparation, and the like, that can optionally be further isolated and/or purified from its native or natural state. A “biological fluid” refers to any a fluid originating from a biological organism. Exemplary biological fluids include, but are not limited to, blood, serum, plasma, and colonic lavage. A biological fluid may be in its natural state or in a modified state by the addition of components such as reagents, or removal of one or more natural constituents (e.g., blood plasma). Methods well-known in the art for collecting, handling, and processing samples, are used in the practice of the present disclosure. The sample may be used directly as obtained from the subject or following pretreatment to modify a characteristic of the sample. Pretreatment may include extraction, concentration, inactivation of interfering components, and/or the addition of reagents. A sample can be from any tissue or fluid from an organism. In some embodiments the sample is from a tissue that is part of, or associated with, a colon polyp of the organism.

The methods described herein can include any suitable method for evaluating gene expression. Determining expression of at least one gene may include, for example, detection of an RNA transcript or portion thereof, and/or an expression product such as a protein or portion thereof. Expression of a gene may be detected using any suitable method known in the art, including but not limited to, detection and/or binding with antibodies, detection and/or binding with antibodies tethered to or associated with an imaging agent, real time RT-PCR, Northern analysis, magnetic particles (e.g., microparticles or nanoparticles), Western analysis, expression reporter plasmids, immunofluorescence, immunohistochemistry, detection based on an activity of an expression product of the gene such as an activity of a protein, any method or system involving flow cytometry, and any suitable array scanner technology. For example, an mRNA transcript of a gene may be detected for determining the expression level of the gene. Based on the sequence information provided by the GenBank™ database entries, the genes can be detected and expression levels measured using techniques well known to one of ordinary skill in the art. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can be also carried out on a DNA array, such as a microarray. The expression level of a protein may be evaluated by immunofluorescence by visualizing cells stained with a fluorescently-labeled protein-specific antibody, Western blot analysis of protein expression, and RT-PCR of protein transcripts. The antibody or fragment thereof may suitably recognize a particular intracellular protein, protein isoform, or protein configuration.

As used herein, an “imaging agent” or “reporter” is any compound or composition that enhances visualization or detection of a target. Any type of detectable imaging agent or reporter may be used in the methods disclosed herein for the detection of an expression product. Exemplary imaging agents and reporters may include, but are not limited to, compounds and compositions comprising magnetic beads, fluorophores, radionuclides, and nuclear stains (e.g., DAPI), and further comprising a targeting moiety for specifically targeting or binding to the target expression product. For example, an imaging agent may include a compound that comprises an unstable isotope (i.e., a radionuclide), such as an alpha- or beta-emitter, or a fluorescent moiety, such as Cy-5, Alexa 647, Alexa 555, Alexa 488, fluorescein, rhodamine, and the like. In some embodiments, suitable radioactive moieties may include labeled polynucleotides and/or polypeptides coupled to the targeting moiety. In some embodiments, the imaging agent may comprise a radionuclide such as, for example, a radionuclide that emits low-energy electrons (e.g., those that emit photons with energies as low as 20 keV). Such nuclides can irradiate the cell to which they are delivered without irradiating surrounding cells or tissues. Non-limiting examples of radionuclides that are can be delivered to cells may include, but are not limited to, ¹³⁷Cs, ¹⁰³Pd, ¹¹¹In, ¹²⁵I, ²¹¹At, ²¹²Bi, and ²¹³Bi, among others known in the art. Further imaging agents may include paramagnetic species for use in MRI imaging, echogenic entities for use in ultrasound imaging, fluorescent entities for use in fluorescence imaging (including quantum dots), and light-active entities for use in optical imaging. A suitable species for MRI imaging is a gadolinium complex of diethylenetriamine pentacetic acid (DTPA). For positron emission tomography (PET), ¹⁸F or ¹¹C may be delivered. Other non-limiting examples of reporter molecules are discussed throughout the disclosure. In some embodiments, determining the expression level of at least one gene includes measuring the expression level of an RNA transcript of the at least one gene, or an expression product thereof. In some embodiments, measuring the expression level of the RNA transcript of the at least one gene, or the expression product thereof, includes using at least one of a PCR-based method, a Northern blot method, a microarray method, and an immunohistochemical method.

The expression level of at least one gene in the sample obtained from the colorectal polyp may be compared to a control value associated with that same gene. A control may include comparison to the level of expression in a control cell, such as a non-cancerous cell, a non-sessile serrated polyp cell, or other normal cell. The control may be from a non-cancerous or non-sessile serrated polyp from the same subject, or it may be from a different subject. Alternatively, a control may include an average range of the level of expression from a population of normal cells. Those skilled in the art will appreciate that a variety of controls may be used. In some embodiments, the control value associated with each gene may be determined by determining the expression level of that gene in one or more control samples, and calculating an average expression level of that gene in the one or more control samples, wherein each control sample is obtained from healthy colonic tissue of the same or a different subject.

The likelihood that the colorectal polyp will develop into colorectal cancer may be predicted based on the relative difference between the expression level and the control value associated with each gene. An increase in the expression level at least one of MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, and ONECUT2 relative to the control value associated with each gene may correlate with an increased likelihood of the colorectal polyp developing into colorectal cancer. The expression of the gene may be increased relative to the expression level of a control by an amount of at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, at least about 100-fold, at least about 150-fold, at least about 200-fold, at least about 250-fold, at least about 300-fold, at least about 350-fold, at least about 400-fold, at least about 450-fold, at least about 500-fold, or at least about 550-fold. In some embodiments, the expression of the gene may be increased relative to the expression level of a control by an amount of at least about 1.5-fold, at least about 5-fold, or at least about 10-fold.

A decrease in the expression level of at least one of SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1 relative to the control value associated with each gene may correlate with an increased likelihood of the colorectal polyp developing into colorectal cancer. The expression of a control may be increased relative to the expression level of the gene by an amount of at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, at least about 100-fold, at least about 150-fold, at least about 200-fold, at least about 250-fold, at least about 300-fold, at least about 350-fold, at least about 400-fold, at least about 450-fold, at least about 500-fold, or at least about 550-fold. In some embodiments, the expression of a control may be increased relative to the expression level of the gene by an amount of at least about 1.5-fold, at least about 2-fold, or at least about 3-fold.

In some embodiments, when the expression level of at least one of MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, and ONECUT2 is greater than the control value, the method further includes diagnosing the polyp as being a sessile serrated adenoma/polyp. In some embodiments, the method further includes diagnosing the subject as having serrated polyposis syndrome, such as when the patient exhibits other symptoms of the syndrome as defined by the WHO (as discussed above). In some embodiments, the method includes increasing the frequency of colonoscopies for the subject.

In some embodiments, when the control value is greater than the expression level of at least one of SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, the method further includes diagnosing the polyp as being a sessile serrated adenoma/polyp. In some embodiments, the method further includes diagnosing the subject as having serrated polyposis syndrome, such as when the patient exhibits other symptoms of the syndrome as defined by the WHO (as discussed above). In some embodiments, the method includes increasing the frequency of colonoscopies for the subject.

In some embodiments, the methods further include determining the expression level of at least one gene selected from MUC5AC, KLK10, TFF1, DUOX2, CDH3, S100P, and GJB5 in the sample obtained from the colorectal polyp, wherein an increase in the expression level of at least one of MUC5AC, KLK10, TFF1, DUOX2, CDH3, S100P, and GJB5 relative to the control value associated with the gene correlates with an increased likelihood of the colorectal polyp developing into colorectal cancer. In some embodiments, the methods further include determining the expression level of at least one gene selected from SLC14A2, CD177, ZG16, and AQP8 in the sample obtained from the colorectal polyp, wherein a decrease in the expression level of at least one of SLC14A2, CD177, ZG16, and AQP8 relative to the control value associated with the gene correlates with an increased likelihood of the colorectal polyp developing into colorectal cancer.

In some aspects, provided are methods of increasing the likelihood of detecting colorectal cancer at an early stage. The methods may include predicting the likelihood that a colorectal polyp in a subject will develop into colorectal cancer according to the method described above, and when there is an increased likelihood that the colorectal polyp will develop into colorectal cancer, the frequency of colonoscopies administered to the subject are increased.

In some aspects, provided are methods for determining the colonoscopy frequency for a patient. Using conventional methods, such as those including histopathology, a number of patients (estimated to be about 20% to about 50%) are being misdiagnosed as having hyperplastic polyps instead of SSA/Ps. Methods described herein including immunohistochemistry diagnostics for SSA/Ps improve cancer screening protocols. Using the methods detailed herein, many patients diagnosed with conventional methods as having hyperplastic polyps (primarily based on standard histology analysis) and recommended to have a follow up surveillance colonoscopy at about 10 years would instead be reclassified as having SSA/Ps and have follow up colonoscopies recommended at earlier time periods such as in about 1, 2, 3, 4, 5 years, or 6 years. For example, a subject having a polyp classified as an SSA/P according to the methods detailed herein and the polyp having diameter of at least about 10 mm would have a subsequent colonoscopy in about 2 years to about 4 years, or about 3 years. For example, a subject having a polyp classified as an SSA/P according to the methods detailed herein and the polyp having of diameter of less than about 5 mm would have a subsequent colonoscopy in about 4 years to about 6 years, or about 5 years. A subject having a polyp classified as an SSA/P according to the methods detailed herein and being of diameter of about 5 mm to about 10 mm would have a subsequent colonoscopy in about 2 years to about 6 years, about 3 to about 5 years, or about 4 years. More frequent colonoscopies may be suggested for patients having multiple SSA/P polyps. By more accurately diagnosing a polyp as a sessile serrated polyp instead of as a hyperplastic polyp, a subject may be more frequently screened by colonoscopy, leading to a reduced incidence of colon cancer and deaths due to colon cancer.

In some aspects, provided are kits for predicting the likelihood that a colorectal polyp in a subject will develop into colorectal cancer. The kits may include at least one primer, each adapted to amplify an RNA transcript of one gene independently selected from MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, and instructions for use. In some embodiments, the kits may further include at least one additional primer, each adapted to amplify an RNA transcript of one gene independently selected from MUC5AC, KLK10, TFF1, DUOX2, CDH3, S100P, GJB5, SLC14A2, CD177, ZG16, and AQP8.

In some aspects, provided are kits for predicting the likelihood that a colorectal polyp in a subject will develop into colorectal cancer. The kits may include one or more probes, each adapted to specifically bind to an RNA transcript, or an expression product thereof, of one gene independently selected from MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, and instructions for use. In some embodiments, the kits may further include one or more additional probes, each adapted to specifically bind to an RNA transcript, or an expression product thereof, of one gene independently selected from MUC5AC, KLK10, TFF1, DUOX2, CDH3, S100P, GJB5, SLC14A2, CD177, ZG16, and AQP8. In some embodiments, at least one probe includes an antibody to an expression product. In some embodiments, at least one probe includes an oligonucleotide complementary to an RNA transcript.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including but not limited to”) unless otherwise noted. All methods described herein can be performed in any suitable order unless otherwise indicated herein or otherwise clearly contradicted by context. The use of any and all examples, or exemplary language (e.g., “such as”) provided herein, is intended merely to illustrate aspects and embodiments of the disclosure and does not limit the scope of the claims.

It will be understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

Also, it is to be understood that the phraseology and terminology used herein is for the purpose of description and should not be regarded as limiting. The use herein of terms such as “comprising,” “including,” “having,” and variations thereof is meant to encompass the items listed thereafter and equivalents thereof as well as additional items. “Comprising” encompasses the terms “consisting of” and “consisting essentially of.” The use of “consisting essentially of” means that the composition or method may include additional ingredients and/or steps, but only if the additional ingredients and/or steps do not materially alter the basic and novel characteristics of the claimed composition or method.

All patents publications and references cited herein are hereby fully incorporated by reference.

While the following examples provide further detailed description of certain embodiments of the invention, they should be considered merely illustrative and not in any way limiting the invention, as defined by the claims.

### EXAMPLES

### Materials and Methods

Patients—

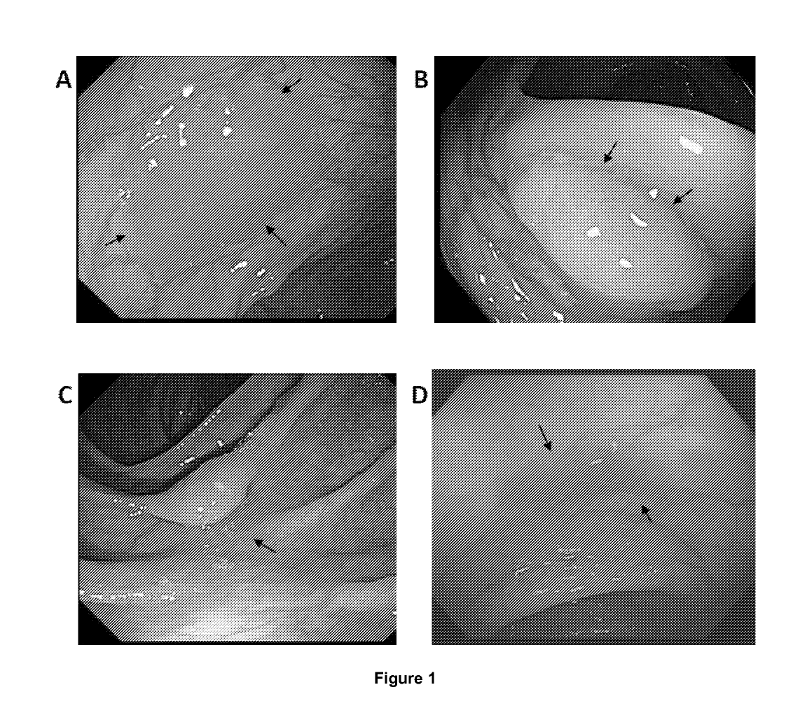

Ethics Statement, all participants provided their written informed consent to participate in this study and all research, including the consent procedure, was approved by the University of Utah Institutional Review Board (IRB). SSA/P and patient matched surrounding uninvolved right colon biopsy specimens were collected from eleven patients with the serrated polyposis syndrome (SPS) seen at the Huntsman Cancer Institute (Table 1, FIG. 1). All polyps (n=21, 10≧1 cm) were collected from the right colon (ascending or proximal transverse) of patients. Normal control colon (right colon; n=10; screening colonoscopy and no polyps) and adenomatous polyp biopsy (n=10; 5-10 mm diameter; right sided; from seven patients) specimens were collected from patients undergoing routine screening colonoscopy at the University of Utah Hospital (Table 4). Biopsy specimens were placed in RNAlater (Invitrogen) immediately following collection and stored at 4° C. overnight prior to total RNA isolation the following day. It was found that this collection method resulted in higher quality RNA than freezing biopsies in liquid nitrogen, storage at −80° C. and subsequent isolation of RNA.

Biospecimens, RNA Isolation, and RNA Sequencing—

All biopsy specimens were collected from the cecum to the splenic flexure (designated right colon) and reviewed by an expert GI pathologist (Table 5). Serrated polyps were classified according to the recent recommendations of the Multi-Society Task Force on Colorectal Cancer for post-polypectomy surveillance that recommended classifying serrated lesions into hyperplastic polyps without subtypes, SSA/P with and without dysplasia, and traditional serrated adenomas (TSAs) that are relatively rare. If a serrated polyp had one or more of the following, size >1 cm, right-sided location, morphologic features of predominantly dilated serrated crypts extending to the mucosal base, or dysmaturation of crypts, it was designated as SSA/P. Other serrated polyps were designated hyperplastic polyps without subtypes. Hyperplastic polyps were not subclassified because of their overlapping histological features and because there is little evidence for any utility in clinical care for subclassifying them. Biopsies taken for RNA sequencing (RNA-seq) analysis were placed immediately into RNAlater® (Invitrogen) and stored at 4° C. overnight prior to total RNA isolation using TRIzol (Invitrogen) the following day. Total RNA was prepared from biopsies of SSA/Ps (n=21, 10≧1 cm diameter) plus patient matched uninvolved colon (n=10) from SPS patients, adenomatous polyps (APs, n=10, 5-10 mm) plus uninvolved colon (n=10) and normal control colon (n=10, screening colonoscopy with no polyps) as described previously. The quantity of RNA recovered from samples was measured by NanoDrop analysis and only samples with a RIN of ≧7 determined by Agilent 2100 Bioanalyzer analysis were used in this study. 5′ capped RNA was isolated, PCR amplified cDNA sequencing libraries prepared using random hexamers following the Illumina RNA sequencing protocol, and single-end 50 bp RNA-seq reads (Illumina HiSeq 2000) performed on seven SSA/Ps, six SPS patient matched uninvolved colon and two normal control colon samples as described previously. Total RNA (RIN of ≧7) from adenomatous polyps and uninvolved colonic mucosa from 17 patients undergoing screening colonoscopy (seven with adenomas and ten without polyps) was used for qPCR analysis (Table 4). Total RNA from SSA/Ps and patient matched uninvolved colonic mucosa from eleven serrated polyposis syndrome (SPS) patients was used for qPCR.

Bioinformatic Analysis—

Sequencing reads were aligned to the GRCh37/Hg19 human reference genome using the Novoalign application (Novocraft). Visualization tracks were prepared for each dataset using the USeqReadCoverage application and viewed using the Integrated Genome Browser (IGB) as described previously. Visualization tracks were scaled using reads per kilobase of gene length per million aligned reads (RPKM) for each Ensemble gene. The USeqOverdispersedRegionScanSeqs (ORSS) application was used to count the reads intersecting exons of each annotated gene and score them for differential expression in uninvolved colon and colon polyps. These p-values were controlled for multiple testing using the Benjamini and Hochberg false discovery method as in prior studies. A normalized ratio was also used to score and filter differentially expressed genes (FDR<0.05, 5 out of 100 false) by their enrichment (≧1.5-fold). The RNA-seq datasets described in this study have been deposited in GEO (GSE46513). Hierarchical clustering of log 2 ratios (polyp/control) comparing RNA-Seq and microarray data (adenomatous polyps GSE8671 and SSA/Ps GSE12514) were performed using Cluster 3.0 and Java treeview software. The fold change and false discovery rate of differentially expressed genes in the microarray datasets were determined using the “multtest” R programming script. Gene set enrichment analysis of differentially expressed gene lists was performed using the Molecular Signatures Database (MSigDB, Broad Institute). Four tubular and three tubulovillous adenomas showing low dysplasia, part of a curated gene set available in the MSigDB, were selected for comparison to SSA/Ps. The adenomas were sex matched (4 females, 3 males), between 1.0 and 3.0 cm in diameter (1.8 mean diameter) and from right (n=3) and left (n=4) colon.

Real-Time PCR (qPCR)—

qPCR analysis was done with the Roche Universal Probe Library and Lightcycler 480 system (Roche Applied Science) on control, uninvolved, SSA/P and AP colon samples. cDNA was prepared from total RNA isolated from polyp and colon specimens and assayed for mRNA levels of selected genes to verify changes observed in the RNA-seq analysis. First-strand cDNA was synthesized using Moloney Murine Leukemia Virus reverse transcriptase (SuperScript III; Invitrogen) with 2 to 5 μg of RNA at 50° C. (60 min) with oligo(dT) primers. Each PCR reaction was carried out in a 96-well optical plate (Roche Applied Science) in a 20 μL reaction buffer containing LightCycler 480 Probes Master Mix, 0.3 μM of each primer, 0.1 μM hydrolysis probe and approximately 50 ng of cDNA (done in triplicate). Triplicate incubations without template were used as negative controls. The qPCR thermo cycling was 95° C. for 5 min, 45 cycles at 95° C. for 10 sec, 60° C. for 30 sec and 72° C. for 1 sec. The relative quantity of each RNA transcript, in polyps compared to controls, was calculated with the comparative Ct (cycling threshold) method using the formula 2^{ΔCt}. β-actin (ACTB) was used as a reference gene.

BRAF Mutation Analysis—

PCR amplicons of BRAF from SSA/Ps, hyperplastic polyps and patient matched uninvolved colon were sequenced for V600E BRAF mutations. Amplicons spanning exons 13-18 of the BRAF gene including the V600E mutation region were prepared (forward primer 5′-AGGGCTCCAGCTTGTATCAC-3′ (SEQ ID NO: 1) and reverse primer 5′-CGATTCAAGGAGGGTTCTGA-3′ (SEQ ID NO: 2), 20 ng of cDNA was amplified with 40 cycles of 95° C. for 30 seconds, 53° C. for 30 sec, and 72° C. for 30 sec) and sequenced in both directions with a Applied Biosystems 3130 Genetic Analyzer.

Immunohistochemistry—

Representative SSA/Ps from patients with serrated polyposis syndrome, sporadic SSA/Ps, hyperplastic polyps, adenomatous polyps and patient matched uninvolved plus normal control colon biopsies were analyzed for VSIG1, MUC17, CTSE, TFF2, and REG4 protein expression by immunohistochemistry. Each polyp and control immunohistochemistry slide was reviewed and scored by an expert GI pathologist (MPB) in a blinded fashion. Polyclonal antigen affinity purified goat, sheep and rabbit primary antibodies were purchased from R&D Systems (anti-VSIG1, cat. #AF4818; anti-CTSE, cat #AF1294; anti-REG4, cat.#AF1379), Sigma-Aldrich (anti-MUC17, cat #HPA031634), ProteinTech (anti-TFF2, cat #12681-1-AP. Four-micron sections of formalin-fixed, paraffin-embedded tissue were mounted on positively charged super-frost/plus slides. Section were deparaffinized with Neo-Clear® Xylene Substitute (Millipore cat. #65351) and rehydrated in a graded series of alcohol to distilled water. Antigen retrieval was performed per the suppliers instructions for each antibody by heating on water bath at 95° C. for 30 min either in 10 mM citrate buffer (pH 6.0) or 10 mM Tris-EDTA Buffer (pH 9.0). Prior to incubation with primary antibodies tissue sections were incubated with a blocking solution of 2.5% normal horse serum (Vector laboratories, cat# S-2012) for 30 min at room temperature. Tissue sections were incubated for 1 hour at room temperature with optimal dilutions of each primary antibody. Samples were washed with 1×PBS (phosphate-buffered saline) and 1×PBS+1% Tween 20. Peroxidase immunostaining was performed, after treatment with BLOXALL™ (Vector Laboratories) endogenous peroxidase blocking solution, using the ImmPRESS polymer system and ImmPACT DAB substrate (Vector Laboratories) per the manufacturer's instructions. Sections were counterstain with hematoxylin QS (Vector Laboratories cat # H-3404). Controls included no primary antibody.

### Example 1

### Gene Expression Analysis

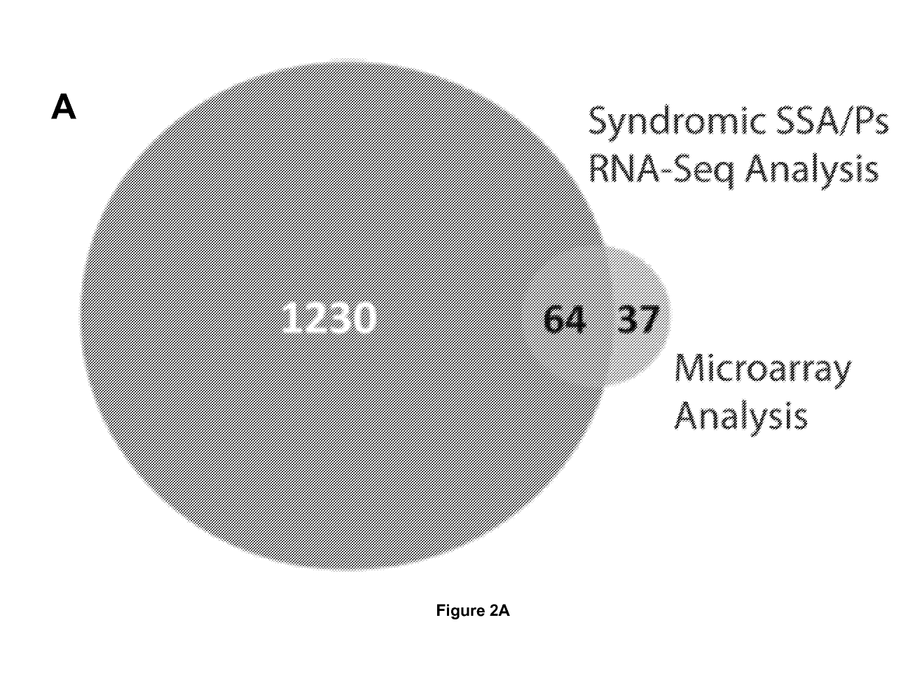

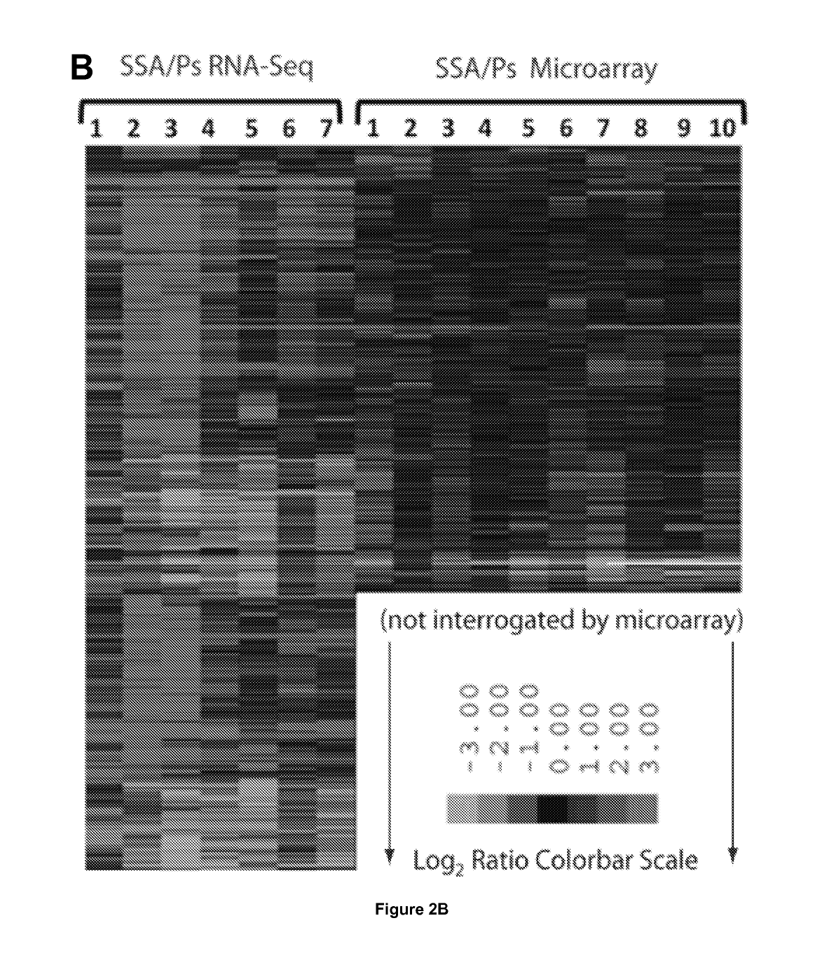

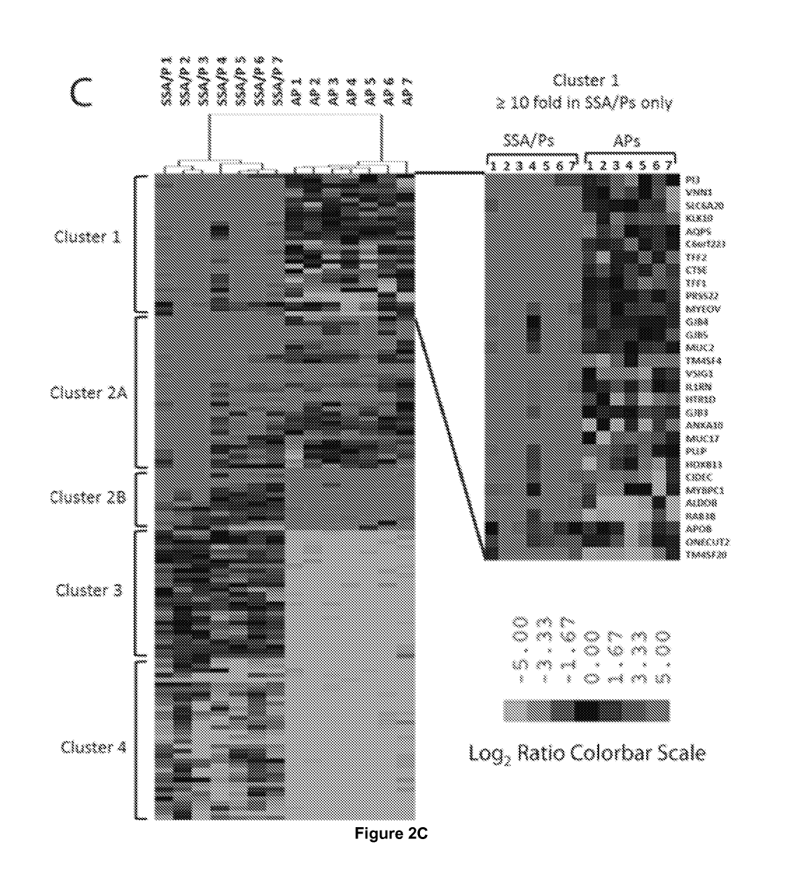

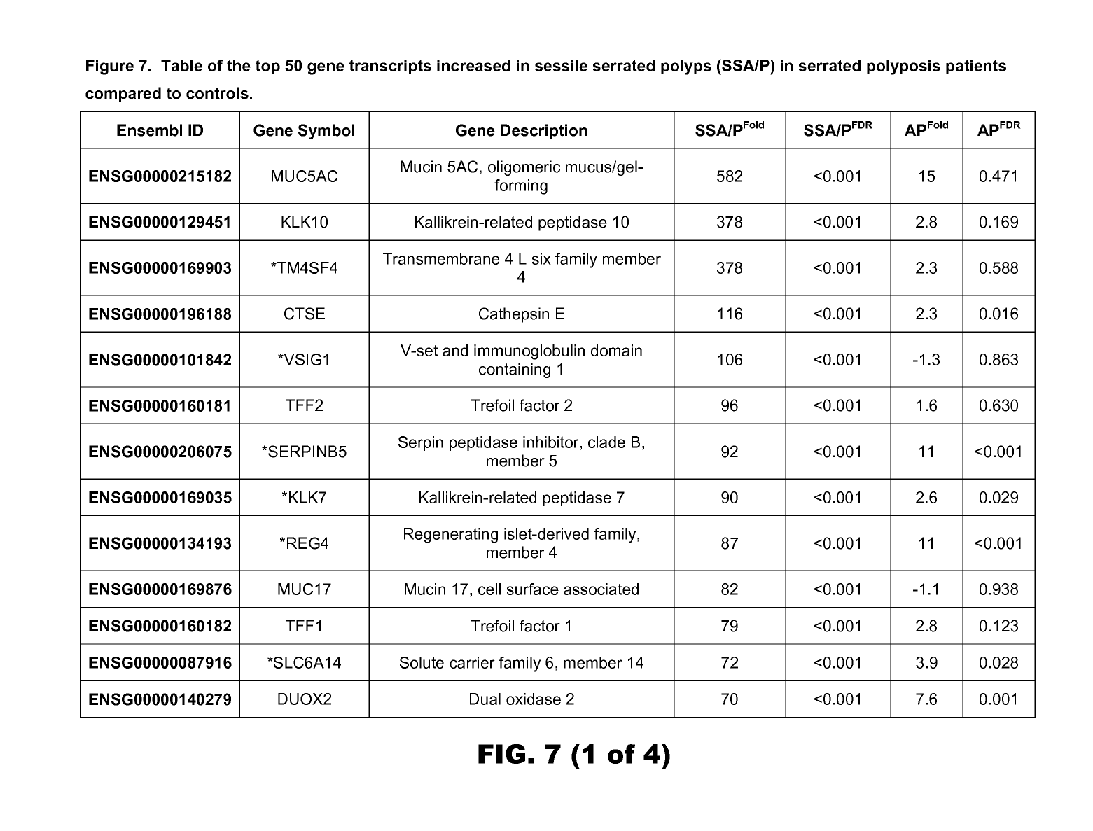

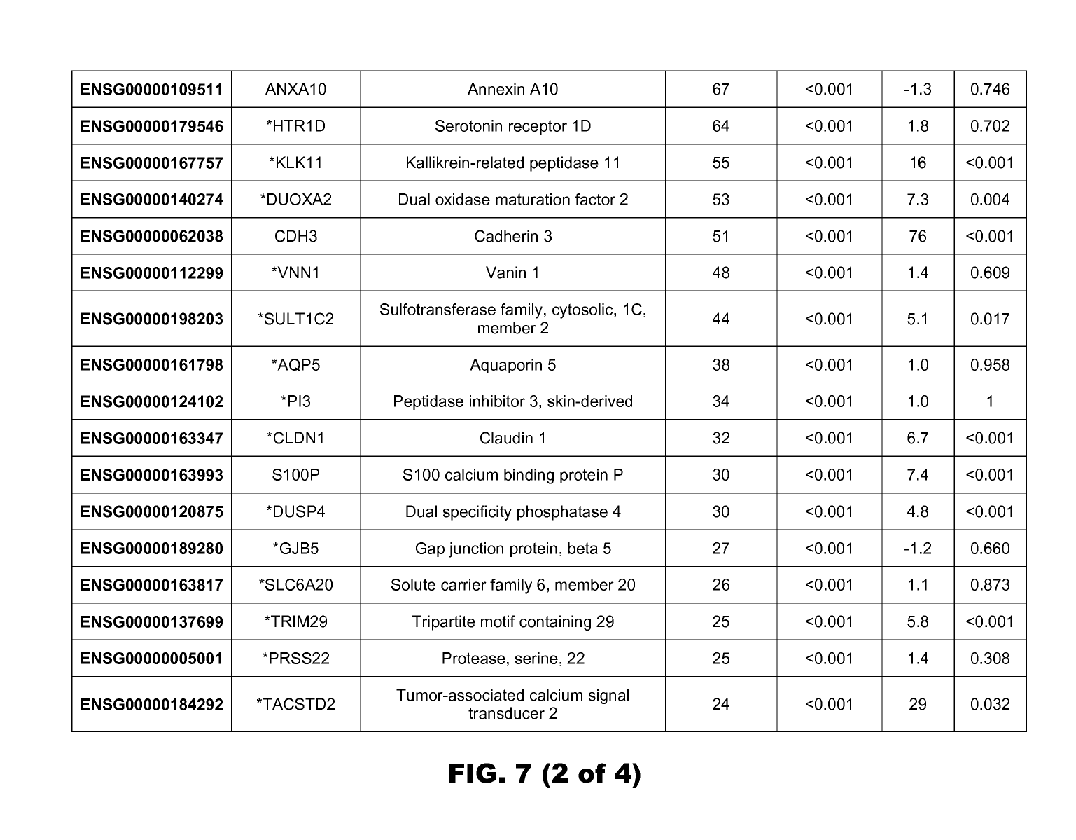

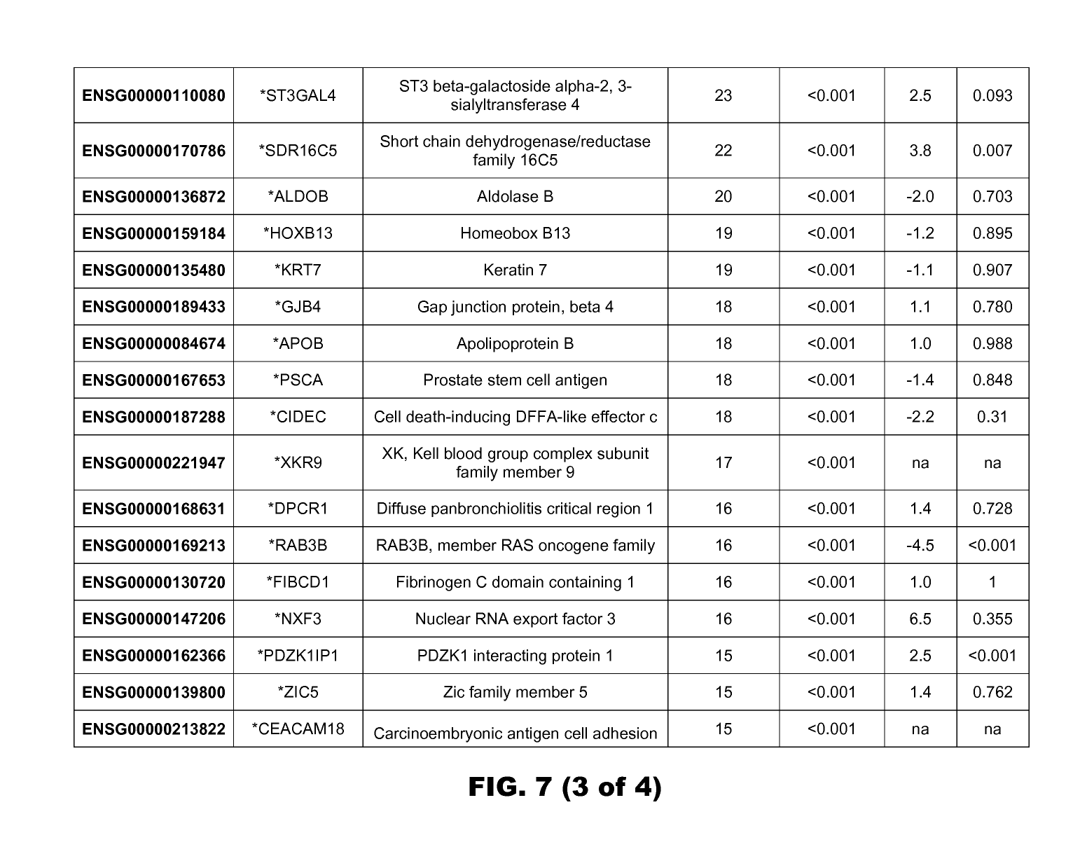

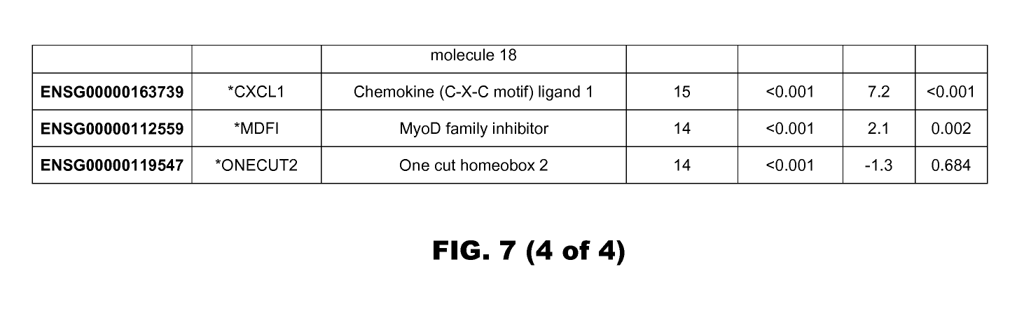

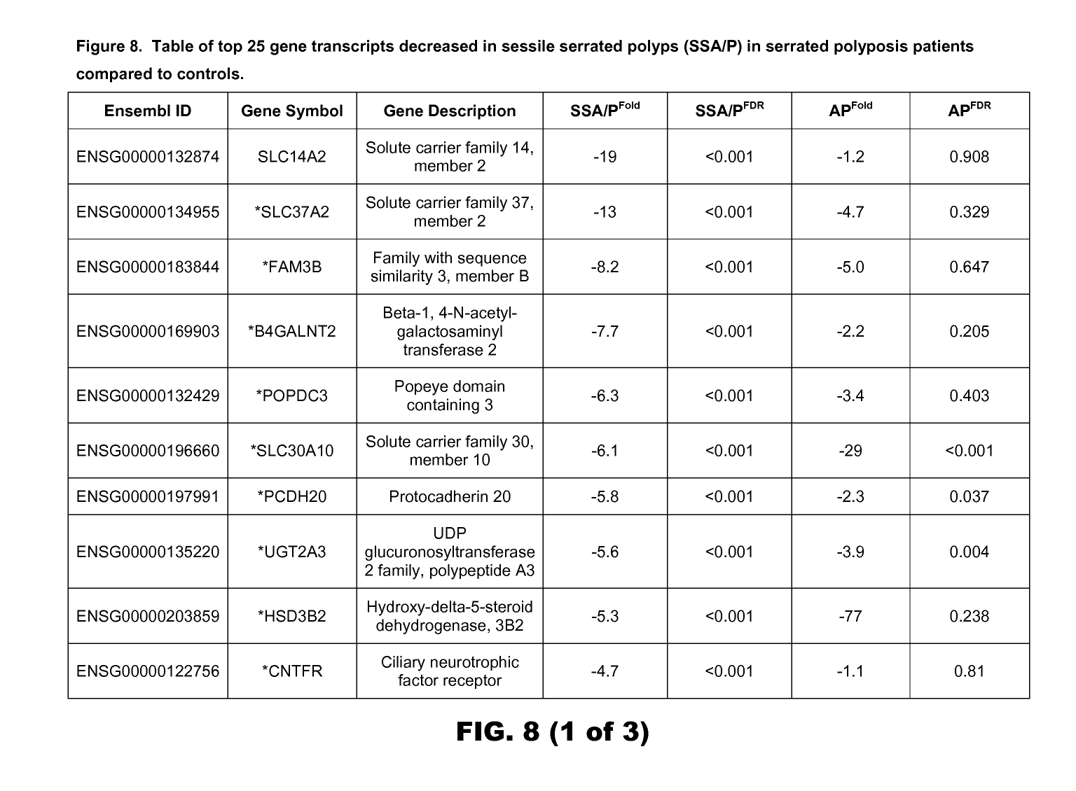

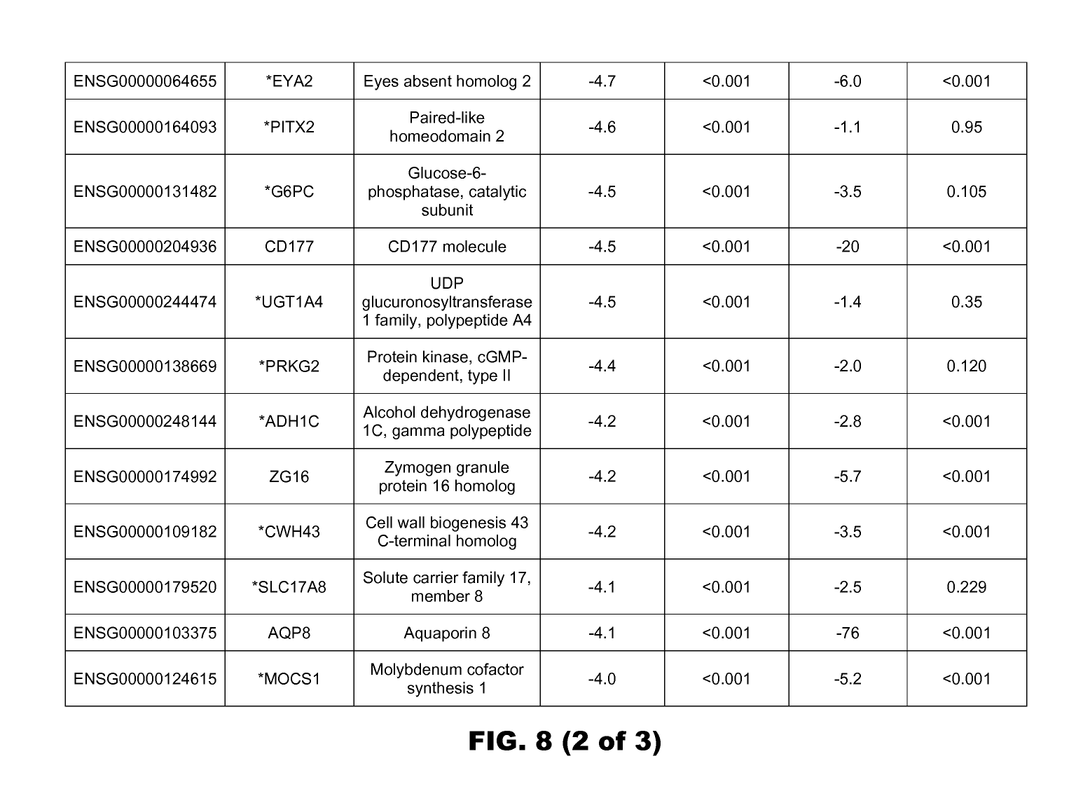

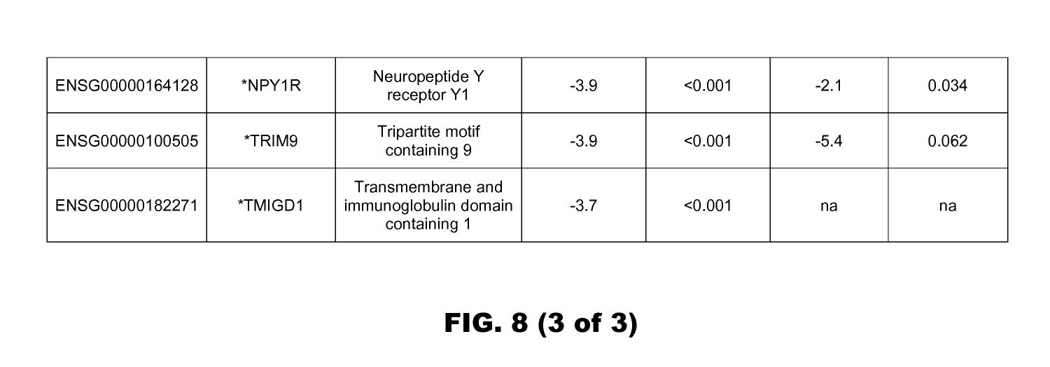

Right-sided (cecum, ascending and transverse colon) SSA/Ps were collected from eleven patients with SPS (Table 1, Table 4, Table 5, FIG. 1) and RNA isolated for RNA-seq and qPCR analysis. A total of seven and twenty-one SSA/Ps were used for RNA-sequencing and qPCR analysis, respectively (Table 5). Bioinformatics analysis of the 5′ capped RNA-seq data identified 1,294 differentially expressed annotated genes [fold change 1.5 and false discovery rate (FDR)<0.05] in SSA/Ps as compared to patient matched uninvolved surrounding colon and normal controls (screening colonoscopy patients with no polyps) (Table 1, FIG. 7, FIG. 8). At least half of the 50 most highly increased genes (all 14-fold, many >50-fold) and 25 most decreased genes were not identified in previous expression microarray studies of SSA/Ps (Table 2, FIG. 8). RNA-seq analysis identified more differentially expressed genes in SSA/Ps (1,294), by an order of magnitude, as compared to a prior microarray analysis (FIG. 2, Panel A). Moreover, 249 of these transcripts were changed ≧5-fold in the RNA-seq analysis as compared to only ten in the array analysis (FIG. 2, Panel B). A microarray study of RNA extracted from SSA/Ps that were formalin fixed and paraffin embedded identified 71 genes that were ≧5 fold in SSA/Ps. The increased number of differentially expressed genes we observed in our RNA-Seq data is consistent with the greater dynamic range of gene expression measurements in RNA-seq analysis.

TABLE 1
Demographics of Patients and Controls for Serrated Polyposis Syndrome. Shown are history
and colonoscopy details of patients with serrated polyposis syndrome. Only polyps with
the serrated histopathology are reported. None of the patients had colon cancer.

| | | | | | | | | | # of | |
|---|---|---|---|---|---|---|---|---|---|---|
| | | | | | Total # of | Total # | # | % | Large | FH |
| | | Age of | | Indication for | Colonos- | of | Proximal | Proximal | Polyps | Colon |
| # | Sex | Diagnosis | Smoking | Colonoscopy | copies | Polyps | Polyps | Polyps | (>1 cm) | Cancer |
| 1 | M | 62 | Never | FH CRC | 5 | 68 | 49 | 72 | 7 | Yes |
| 2 | M | 33 | Never | Hematochezia | 5 | 38 | 14 | 36 | 0 | Yes |
| 3 | F | 24 | Never | Diarrhea | 7 | 33 | 16 | 48 | 7 | No |
| 4 | F | 28 | Never | Hematochezia | 3 | 18 | 14 | 77 | 5 | No |
| 5 | M | 18 | Never | Abd pain | 6 | 91 | 22 | 24 | 0 | No |
| 6 | F | 26 | Current | Hematochezia | 6 | 67 | 54 | 80 | 0 | No |
| 7 | M | 51 | Current | Screening | 2 | 15 | 10 | 66 | 7 | Yes |
| 8 | M | 71 | Ex-smoker | Screening | 6 | 81 | 28 | 34 | 0 | Yes |
| 9 | M | 27 | Ex-smoker | Hematochezia | 2 | 44 | 8 | 18 | 1 | No |
| 10 | M | 25 | Ex-smoker | Hematochezia | 2 | 30 | 19 | 63 | 2 | No |
| 11 | F | 27 | Never | FH CRC | 3 | 23 | 10 | 43 | 1 | Yes |

FH = Family History.

TABLE 4
Demographics of Patients and Controls for Serrated Polyposis Syndrome.
Shown are history and colonoscopy details of patients with serrated
polyposis syndrome. Only polyps with the serrated histopathology
are reported. None of the patients had colon cancer.

| | | | Controls | | |
|---|---|---|---|---|---|
| Adenomatous Polyps | | | (Screening colonoscopy, no polyps) | | |
| # of patient | Age | Sex | # of patient | Age | Sex |
| 1 | 80 | M | 1 | 63 | M |
| 2 | 66 | M | 2 | 54 | F |
| 2 | 66 | M | 3 | 46 | F |
| 2 | 66 | M | 4 | 50 | F |
| 3 | 44 | M | 5 | 50 | M |
| 3 | 44 | M | 6 | 68 | M |
| 4 | 53 | F | 7 | 61 | F |
| 5 | 64 | M | 8 | 48 | M |
| 6 | 53 | F | 9 | 58 | M |
| 7 | 50 | M | 10 | 50 | M |

FH = Family History.

TABLE 5
Phenotype of SSA/Ps from patients with serrated polyposis
syndrome (SPS) that were analyzed by RNA-Seq and qPCR.

| | | Size | | | | |
|---|---|---|---|---|---|---|
| | | Diameter | | | | |
| Patient | Sample | (mm) | Location | Pathology | RNA-seq | qPCR |
| 1 | 1A | 10 | AC | SSA/P | Yes | Yes |
| 1 | 1B | 10 | TC | SSA/P | No | Yes |
| 2 | 2A | 6 | AC | SSA/P | No | Yes |
| 2 | 2B | 4 | TC | No | No | Yes |
| 3 | 3A | 8 | AC | SSA/P | Yes | Yes |
| 3 | 3B | 12 | AC | SSA/P | Yes | Yes |
| 4 | 4 | 15 | AC | SSA/P | Yes | Yes |
| 5 | 5A | 4 | AC | No | Yes | Yes |
| 5 | 5B | 5 | AC | No | No | Yes |
| 6 | 6A | 4 | AC | SSA/P | Yes | Yes |
| 6 | 6B | 4 | TC | No | No | Yes |
| 6 | 6C | 3 | AC | No | Yes | Yes |
| 7 | 7A | 12 | AC | SSA/P | No | Yes |
| 7 | 7B | 15 | TC | SSA/P | No | Yes |
| 8 | 8A | 8 | Cecum | SSA/P | No | Yes |
| 8 | 8B | 12 | AC | SSA/P | No | Yes |
| 9 | 9A | 5 | Cecum | SSA/P | No | Yes |
| 9 | 9B | 15 | AC | SSA/P | No | Yes |
| 9 | 9C | 6 | TC | SSA/P | No | Yes |
| 10 | 10 | 10 | TC | SSA/P | No | Yes |
| 11 | 11 | 12 | AC | SSA/P | No | Yes |

AC = Ascending colon; TC = Transverse Colon.

TABLE 2
Top 50 gene transcripts increased by RNA sequencing in sessile serrated polyps (SSA/P) in serrated polyposis
patients compared to controls. Fold change is reported for seven right-sided sessile serrated polyps,
from five serrated polyposis patients (age 26-62 years, 3 female and 2 male), compared to surrounding
uninvolved colon and normal colon from healthy volunteers (controls, n = 8). Fold-change (Fold) and
false discovery rate (FDR) for specific gene sequencing reads are provided (see Methods). The fold change
and FDR in sex matched adenomatous polyps (AP) (age 55-79 years, three right-sided and four left-sided)
with low dysplasia compared to uninvolved colon (n = 7) from a previous microarray study are provided
(Sabates-Bellver, et al., 2007). Genes with an asterisk have not been previously reported to be differentially
expressed in SSA/Ps. “na” denotes transcripts not analyzed in the microarray study.

| | Gene | | | | | |
|---|---|---|---|---|---|---|
| Ensembl ID | Symbol | Gene Description | SSA/P^{Fold} | SSA/P^{FDR} | AP^{Fold} | AP^{FDR} |
| ENSG00000215182 | MUC5AC | Mucin 5AC, oligomeric | 582 | <0.001 | 15 | 0.471 |
| | | mucus/gel-forming | | | | |
| ENSG00000129451 | KLK10 | Kallikrein-related peptidase 10 | 378 | <0.001 | 2.8 | 0.169 |
| ENSG00000169903 | TM4SF4 | Transmembrane 4 L six | 378 | <0.001 | 2.3 | 0.588 |
| | | family member 4 | | | | |
| ENSG00000196188 | CTSE | Cathepsin E | 116 | <0.001 | 2.3 | 0.016 |
| ENSG00000101842 | *VSIG1 | V-set and immunoglobulin | 106 | <0.001 | −1.3 | 0.863 |
| | | domain containing 1 | | | | |
| ENSG00000160181 | TFF2 | Trefoil factor 2 | 96 | <0.001 | 1.6 | 0.630 |
| ENSG00000206075 | SERPINB5 | Serpin peptidase inhibitor, | 92 | <0.001 | 11 | <0.001 |
| | | clade B, member 5 | | | | |
| ENSG00000169035 | KLK7 | Kallikrein-related peptidase 7 | 90 | <0.001 | 2.6 | 0.029 |
| ENSG00000134193 | REG4 | Regenerating islet-derived | 87 | <0.001 | 11 | <0.001 |
| | | family, member 4 | | | | |
| ENSG00000169876 | MUC17 | Mucin 17, cell surface | 82 | <0.001 | −1.1 | 0.938 |
| | | associated | | | | |
| ENSG00000160182 | TFF1 | Trefoil factor 1 | 79 | <0.001 | 2.8 | 0.123 |
| ENSG00000087916 | *SLC6A14 | Solute carrier family 6, | 72 | <0.001 | 3.9 | 0.028 |
| | | member 14 | | | | |
| ENSG00000140279 | *DUOX2 | Dual oxidase 2 | 70 | <0.001 | 7.6 | 0.001 |
| ENSG00000109511 | ANXA10 | Annexin A10 | 67 | <0.001 | −1.3 | 0.746 |
| ENSG00000179546 | *HTR1D | Serotonin receptor 1D | 64 | <0.001 | 1.8 | 0.702 |
| ENSG00000167757 | KLK11 | Kallikrein-related peptidase 11 | 55 | <0.001 | 16 | <0.001 |
| ENSG00000140274 | *DUOXA2 | Dual oxidase maturation | 53 | <0.001 | 7.3 | 0.004 |
| | | factor 2 | | | | |
| ENSG00000062038 | CDH3 | Cadherin 3 | 51 | <0.001 | 76 | <0.001 |
| ENSG00000112299 | VNN1 | Vanin 1 | 48 | <0.001 | 1.4 | 0.609 |
| ENSG00000198203 | *SULT1C2 | Sulfotransferase family, | 44 | <0.001 | 5.1 | 0.017 |
| | | cytosolic, 1C, member 2 | | | | |
| ENSG00000161798 | AQP5 | Aquaporin 5 | 38 | <0.001 | 1.0 | 0.958 |
| ENSG00000124102 | *PI3 | Peptidase inhibitor 3, skin- | 34 | <0.001 | 1.0 | 1 |
| | | derived | | | | |
| ENSG00000163347 | CLDN1 | Claudin 1 | 32 | <0.001 | 6.7 | <0.001 |
| ENSG00000163993 | *S100P | S100 calcium binding protein P | 30 | <0.001 | 7.4 | <0.001 |
| ENSG00000120875 | *DUSP4 | Dual specificity phosphatase 4 | 30 | <0.001 | 4.8 | <0.001 |
| ENSG00000189280 | GJB5 | Gap junction protein, beta 5 | 27 | <0.001 | −1.2 | 0.660 |
| ENSG00000163817 | *SLC6A20 | Solute carrier family 6, | 26 | <0.001 | 1.1 | 0.873 |
| | | member 20 | | | | |
| ENSG00000137699 | *TRIM29 | Tripartite motif containing 29 | 25 | <0.001 | 5.8 | <0.001 |
| ENSG00000005001 | *PRSS22 | Protease, serine, 22 | 25 | <0.001 | 1.4 | 0.308 |
| ENSG00000184292 | TACSTD2 | Tumor-associated calcium | 24 | <0.001 | 29 | 0.032 |
| | | signal transducer 2 | | | | |
| ENSG00000110080 | *ST3GAL4 | ST3 beta-galactoside alpha- | 23 | <0.001 | 2.5 | 0.093 |
| | | 2,3-sialyltransferase 4 | | | | |
| ENSG00000170786 | SDR16C5 | Short chain | 22 | <0.001 | 3.8 | 0.007 |
| | | dehydrogenase/reductase | | | | |
| | | family 16C5 | | | | |
| ENSG00000136872 | *ALDOB | Aldolase B | 20 | <0.001 | −2.0 | 0.703 |
| ENSG00000159184 | *HOXB13 | Homeobox B13 | 19 | <0.001 | −1.2 | 0.895 |
| ENSG00000135480 | KRT7 | Keratin 7 | 19 | <0.001 | −1.1 | 0.907 |
| ENSG00000189433 | *GJB4 | Gap junction protein, beta 4 | 18 | <0.001 | 1.1 | 0.780 |
| ENSG00000084674 | *APOB | Apolipoprotein B | 18 | <0.001 | 1.0 | 0.988 |
| ENSG00000167653 | *PSCA | Prostate stem cell antigen | 18 | <0.001 | −1.4 | 0.848 |
| ENSG00000187288 | *CIDEC | Cell death-inducing DFFA- | 18 | <0.001 | −2.2 | 0.31 |
| | | like effector c | | | | |
| ENSG00000221947 | *XKR9 | XK, Kell blood group | 17 | <0.001 | na | na |
| | | complex subunit family | | | | |
| | | member 9 | | | | |
| ENSG00000168631 | *DPCR1 | Diffuse panbronchiolitis | 16 | <0.001 | 1.4 | 0.728 |
| | | critical region 1 | | | | |
| ENSG00000169213 | *RAB3B | RAB3B, member RAS | 16 | <0.001 | −4.5 | <0.001 |
| | | oncogene family | | | | |
| ENSG00000130720 | FIBCD1 | Fibrinogen C domain | 16 | <0.001 | 1.0 | 1 |
| | | containing 1 | | | | |
| ENSG00000147206 | NXF3 | Nuclear RNA export factor 3 | 16 | <0.001 | 6.5 | 0.355 |
| ENSG00000162366 | *PDZK1IP1 | PDZK1 interacting protein 1 | 15 | <0.001 | 2.5 | <0.001 |
| ENSG00000139800 | ZIC5 | Zic family member 5 | 15 | <0.001 | 1.4 | 0.762 |
| ENSG00000213822 | *CEACAM18 | Carcinoembryonic antigen | 15 | <0.001 | na | na |
| | | cell adhesion molecule 18 | | | | |
| ENSG00000163739 | *CXCL1 | Chemokine (C-X-C motif) | 15 | <0.001 | 7.2 | <0.001 |
| | | ligand 1 | | | | |
| ENSG00000112559 | *MDFI | MyoD family inhibitor | 14 | <0.001 | 2.1 | 0.002 |
| ENSG00000119547 | ONECUT2 | One cut homeobox 2 | 14 | <0.001 | −1.3 | 0.684 |

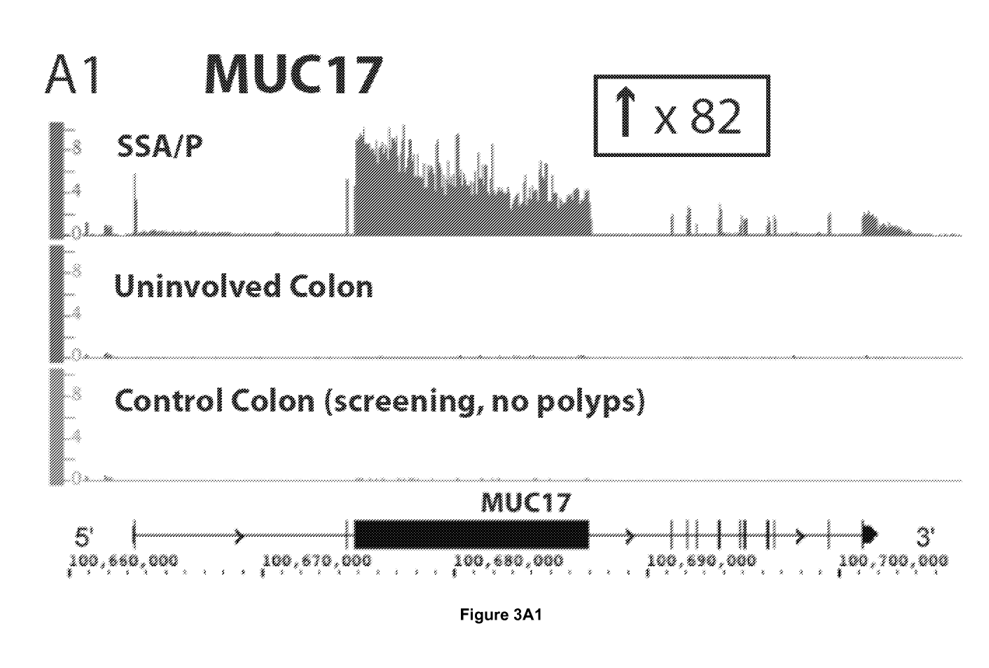

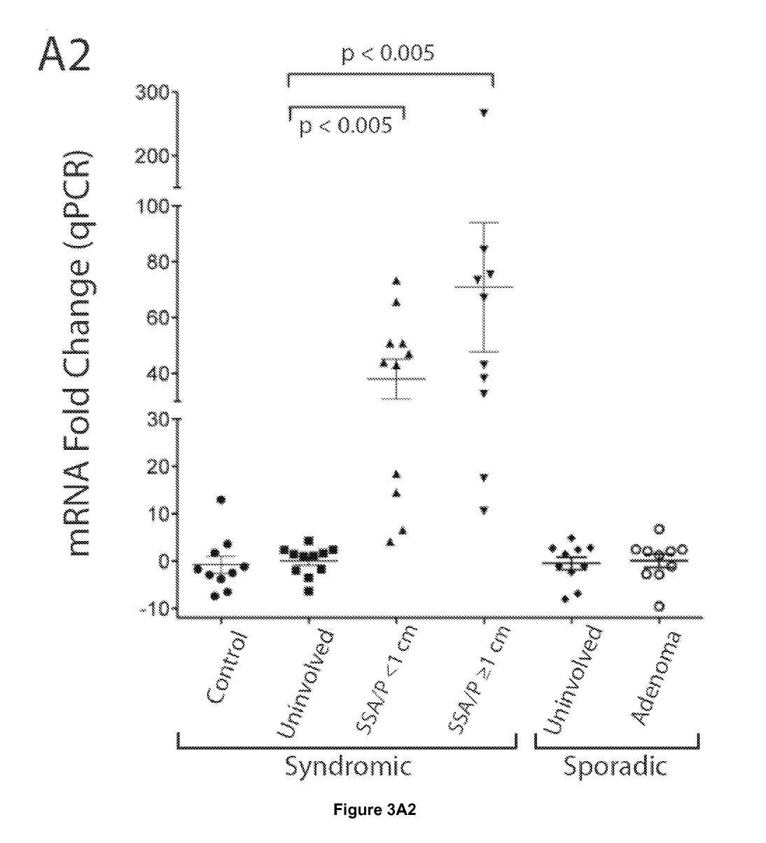

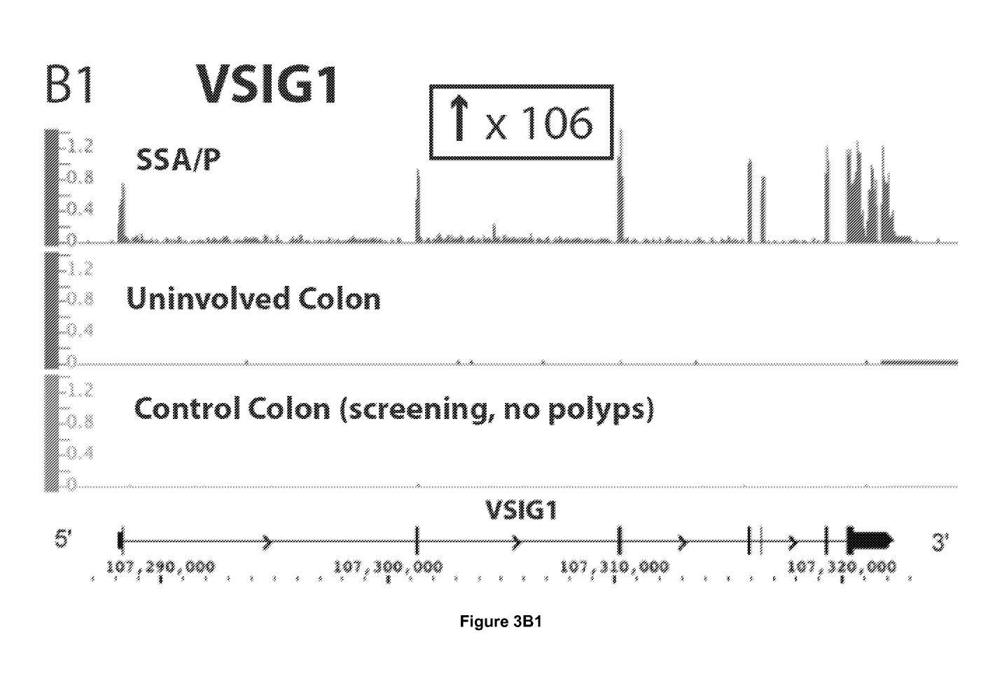

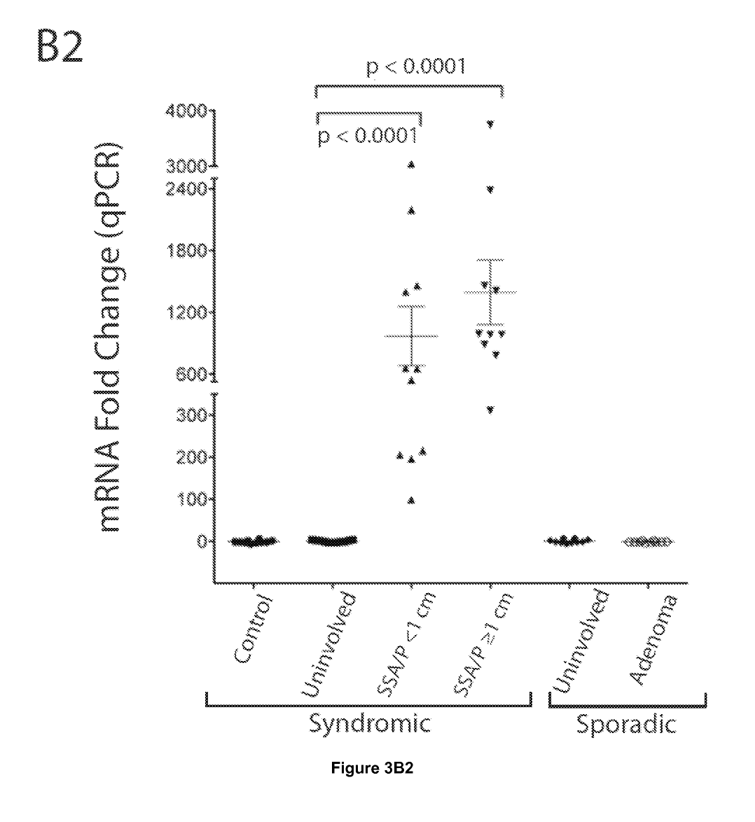

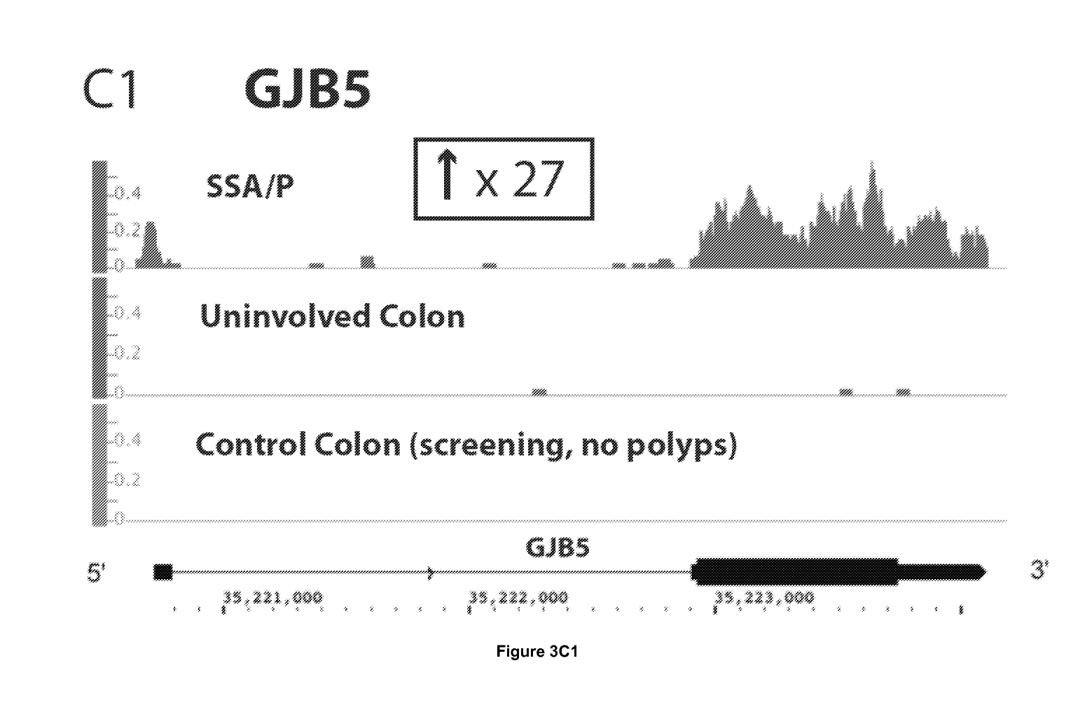

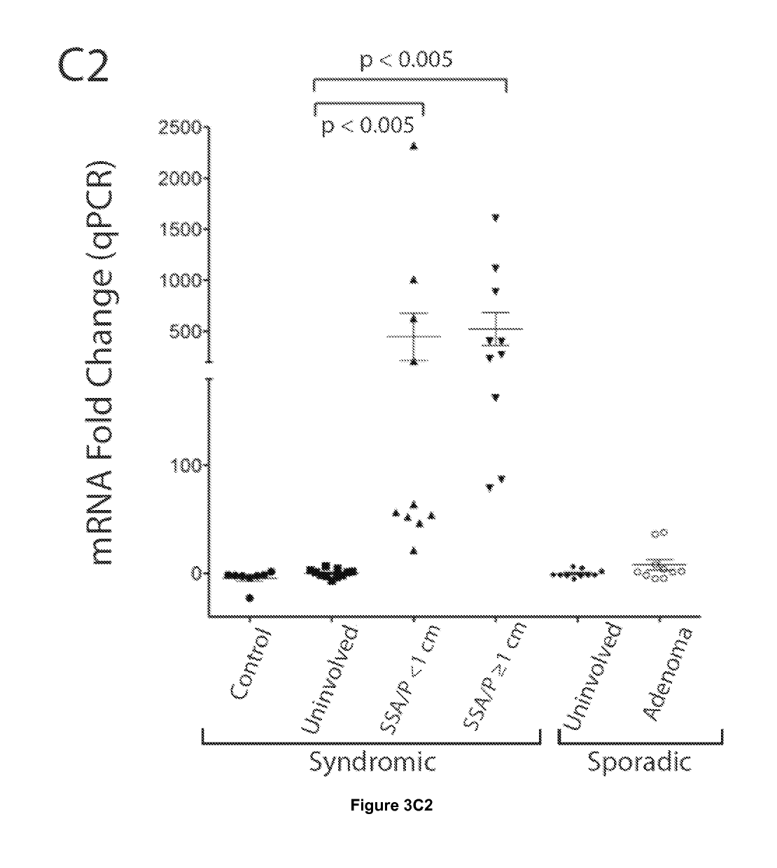

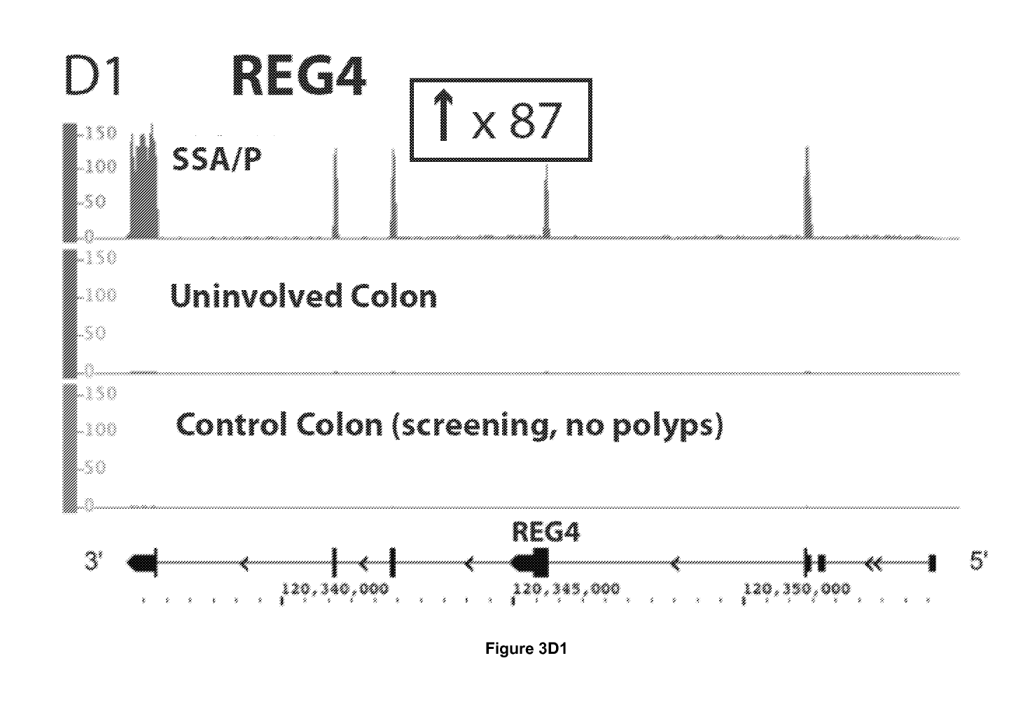

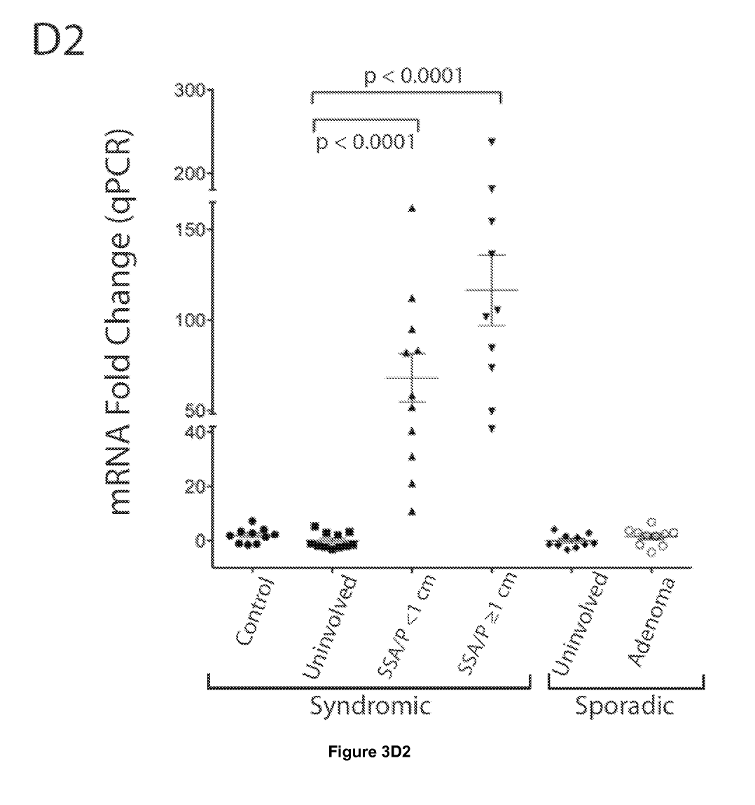

Differentially expressed genes in the RNA-seq SSA/Ps dataset were compared to adenomatous polyp data that is part of a curated gene set available in the Molecular Signature Database at the Broad Institute. Differentially expressed genes from an equal number of adenomatous polyps from sex matched patients (n=7, three men & four women) with low dysplasia were used for comparison. To identify genes that were highly expressed in SSA/Ps, but not in adenomatous polyps, we did hierarchical clustering analysis of 142 differentially expressed genes (>10-fold, FDR<0.05) from each dataset (FIG. 2, Panel C). Approximately 60% of the 75 most highly differentially expressed genes in SSA/Ps (50 increased and 25 decreased) were not differentially expressed in adenomatous polyps relative to controls (Table 2 & 6). Genes that were highly increased (≧10-fold, 30 genes) in SSA/Ps (FIG. 2, Panel C), but not significantly increased in adenomatous polyps, were analyzed by gene set enrichment (GSEA) analyses. Three biological pathways overrepresented in SSA/Ps were mucosal integrity (digestion), cell communication (adhesion) and epithelial cell development. Secreted trefoil factor and mucin genes associated with mucosal integrity that were increased included, mucin 5AC (MUC5AC,↑582-fold), cathepsin E (CTSE,↑116-fold), trefoil factor 2 (TFF2,↑96-fold), trefoil factor 1 (TFF1, ↑79-fold) and mucin 2 (MUC2,↑14-fold) (FIGS. 7-9). A membrane bound regulatory mucin, Mucin 17 (MUC17,↑82-fold), was also highly increased in SSA/Ps (FIG. 3, Panel A1).

RT-qPCR analysis of twenty-one right sided SSA/Ps and uninvolved colon from SPS patients, ten right sided adenomatous polyps plus uninvolved colon and ten right sided normal control biopsies were done to verify the RNA-seq findings of selected genes. qPCR analysis verified the marked overexpression of MUC17 (38-fold in small; 71-fold in large SSA/Ps) in SSA/Ps compared to adenomatous polyps and controls (FIG. 3, Panel A2). The gene for a cell adhesion protein, membrane associated V-set and immunoglobulin domain containing 1 gene (VSIG1), that was markedly increased by RNA-seq analysis (↑106-fold) was also highly increased in SSA/Ps by qPCR analysis (969-fold in small; 1,393-fold in large SSA/Ps) (FIG. 3, Panel B). Expression of several gap junction (connexin) genes were also highly increased in SSA/Ps including gap junction protein beta-5 (GJB5 or connexin 31.1,↑27-fold), gap junction protein, beta 3 (GJB3 or connexin 31, ↑14-fold), gap junction protein, and beta 4 (GJB4 or connexin 30.3,↑18-fold) (FIG. 3, Panel C; Table 2, FIG. 8). qPCR analysis verified the increase in GJB5 in SSA/Ps (446 and 523-fold in small and large polyps, respectively) relative to adenomatous polyps and controls (FIG. 3, Panel C). Three tetraspanin genes, encoding proteins that interact with cell adhesion molecules and growth factor receptors, transmembrane 4 L six family member 4 (TM4SF4,↑378-fold), transmembrane 4 L six family member 20 (TM4SF20,↑14-fold) and plasmolipin (PLLP,↑11-fold) were highly increased in SSA/Ps.

Shown in Table 7 are data for four gene transcripts uniquely and consistently upregulated in Sessile Serrated Polyps (SSA/Ps) compared to hyperplastic polyps, indicating that CTSE, VSIG1, TFF2, and MUC17 are expressed in low levels in hyperplastic polyps, while they are overexpressed in SSA/Ps relative to basal levels such as wherein no polyps are present.

TABLE 7
Gene Transcripts Uniquely Upregulated in Sessile Serrated Polyps (SSA/Ps). Shown
are details for CTSE, VSIG1, TFF2, and MUC17 mRNA transcripts in sessile serrated
polyps (SSA/Ps) of serrated polyposis patients compared to control colon. Fold
change is reported for 7 right-sided SSA/Ps (four > 1 cm), from 5 serrated
polyposis patients (age range 26-62, 3 female and 2 male), compared to surrounding
uninvolved colon and normal colon from healthy volunteers (n = 8). False
discovery rate (FDR) is shown on the right. The fold change and FDR for 15 hyperplastic
polyps (HPs) from screening colonoscopy patients compared to uninvolved and normal
colon (n = 15) is also shown. In each case, the fold change in SSA/Ps is
an order of magnitude greater than that observed in HPs.

| | Gene | Gene | | | | |
|---|---|---|---|---|---|---|
| Ensembl ID | Symbol | Description | SSA/P^{Fold} | SSA/P^{FDR} | HP^{Fold} | HP^{FDR} |
| ENSG00000196188 | CTSE | Cathepsin E | 116 | <0.001 | 7.6 | <0.001 |
| ENSG00000101842 | VSIG1 | V-set and | 106 | <0.001 | 5.1 | <0.001 |
| | | immunoglobulin | | | | |
| | | domain | | | | |
| | | containing 1 | | | | |
| ENSG00000160181 | TFF2 | Trefoil factor 2 | 96 | <0.001 | 4.9 | <0.001 |
| ENSG00000169876 | MUC17 | Mucin 17, cell | 82 | <0.001 | 3.1 | <0.001 |
| | | surface associated | | | | |

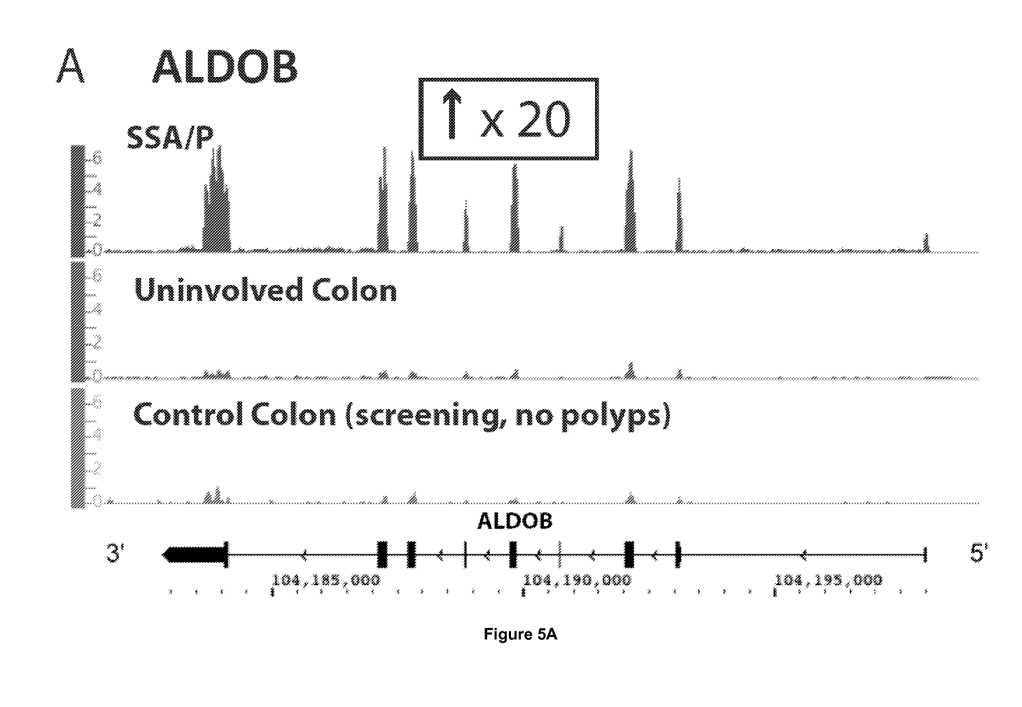

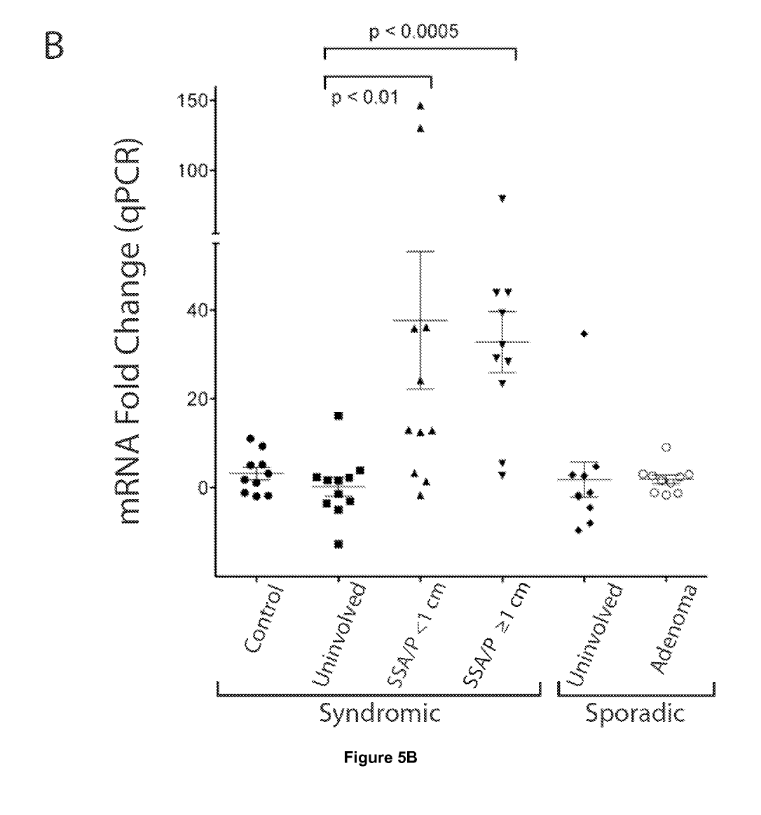

Other highly expressed genes in SSA/Ps, reported to be increased in inflammatory or neoplastic conditions of the colon, included regenerating islet-derived family member 4 (REG4,↑87-fold; FIG. 3, Panel D), kallikrein 10 (KLK10,↑378-fold), aquaporin 5 (AQP5,↑38-fold), myeloma overexpressed (MYEOV,↑14-fold) and aldolase B (ALDOB or fructose-bisphosphate aldolase B, ↑20-fold) (Table 2, FIG. 8). qPCR analysis confirmed the increase in ALDOB (33 to 38-fold) in SSA/Ps (FIG. 5). Increased expression of REG4 was reported in gastric intestinal metaplasia and colonic adenomatous polyps suggesting a role in premalignant lesions. qPCR analysis verified the increase in REG4 (68 to 116-fold) in SSA/Ps compared to controls (FIG. 3, Panel D). The transcription factors homeobox B13 (HOXB13,↑19-fold) and one cut homeobox 2 (ONECUT2,↑14-fold), critical in epithelial cell development and differentiation, both had >10-fold increases in their mRNA in SSA/Ps by RNA-seq analysis (Table 2, FIG. 8). Neither of these transcription factors was significantly expressed in controls (0.006-0.03 RPKM) and prior gene array studies did not show significant changes in adenomatous polyps as compared to controls.

### Example 2

### BRAF Mutation Analysis

BRAF in SSA/Ps was amplified by PCR and sequenced since T to A mutations in codon 600 resulting in a valine to glutamic acid (V600E) amino acid change with increased kinase activity have been reported in SSA/Ps (Materials and Methods). PCR amplicons of the BRAF gene from twenty SSA/Ps (twelve patients), ten hyperplastic polyps, and patient matched uninvolved control specimens were sequenced. Consistent with other reports, 60% of SSA/Ps had V600E mutations in BRAF while no mutations were observed in hyperplastic polyps and controls (Table 6).

TABLE 6
BRAF V600E mutations in SSA/Ps and uninvolved colon from patients with
serrated polyposis syndrome. Sequencing of a 700 bp PCR amplicon of BRAF, that included
codon 600, was done on samples (20 SSA/Ps and patient matched uninvolved controls) from
twelve serrated polyposis patients. PCR products were sequenced (both strands) using an
Applied Biosystems 3130 Genetic Analyzer and mutations were identified using Mutation
Surveyor software (see SI Materials and Methods). Hyperplastic polyps and patient matched
uninvolved colon (five patients) were also analyzed and showed no V600E BRAF mutations.

| Tissue | Number of Samples | BRAF V600E (%) |
|---|---|---|
| Patient matched uninvolved colon | 16 | 0 (0) |
| SSA/Ps | 20 | 12 (60) |
| Hyperplastic polyps | 10 | 0 (0) |
| Size | | |
| Large SSA/Ps (≧1 cm) | 10 | 7 (70) |
| Small SSA/Ps (<1 cm) | 10 | 5 (50) |

### Example 3

### Immunohistochemistry

Immunohistochemistry (IHC) for VSIG1, MUC17, CTSE, TFF2, and REG4 in a panel of routinely formalin fixed and paraffin embedded SSA/Ps, hyperplastic polyps, adenomatous polyps, and control specimens was done to further validate the RNA-seq data, identify the cell types involved in overexpression, and to investigate their potential diagnostic utility for differentiating SSA/Ps from other polyps. All control and polyp specimens were reviewed by an expert GI pathologist (MPB).

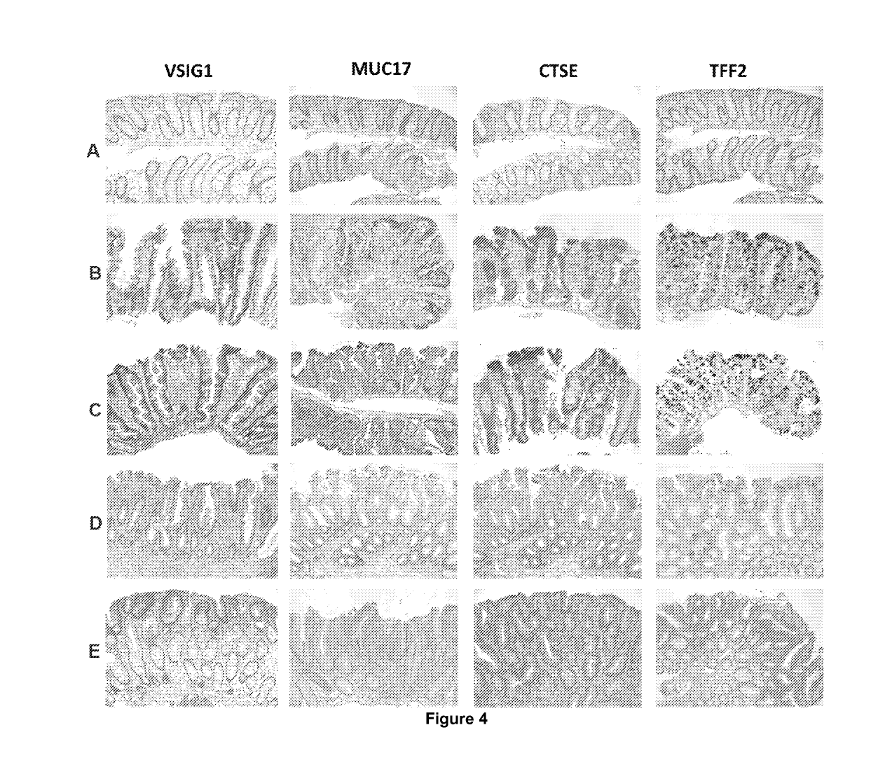

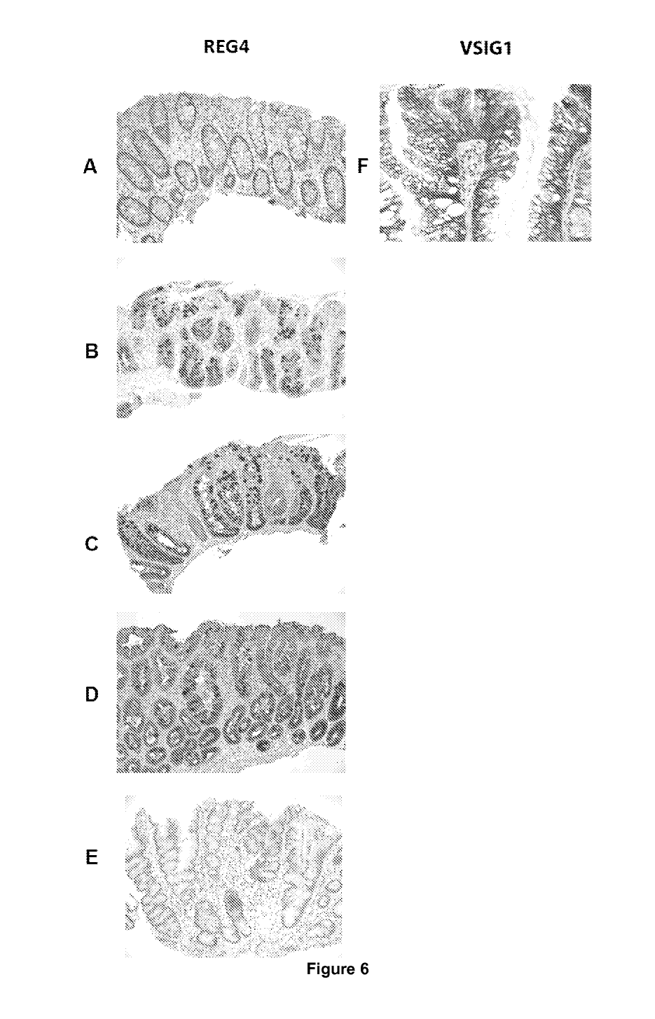

Intense and unique patterns of staining were found for VSIG1, MUC17, CTSE and TFF2 that differentiated SSA/Ps from other polyps and controls (FIG. 4, Table 2). Immunostaining for VSIG1 was absent in control colon (FIG. 4, Panel A), whereas with both syndromic (Panel B) and sporadic SSA/Ps (Panel C) there was intense (3 to 4+, on a scale of 0-4, 4 being highest) staining of most epithelial cell junctions (>70%) in both the luminal surface and along the crypt axis (FIG. 4, Table 3, FIG. 6). Hyperplastic polyps (Panel D) showed trace to 1+ immunostaining in ˜25% of epithelial cells. Adenomatous polyps (line E) showed trace or no staining. Immunostaining for MUC17 in the cytoplasm of control colon epithelium was trace, whereas with SSA/Ps there was a distinctive pattern of staining that was 2 to 3+ in the cytoplasm of approximately 60% of epithelial cells and most pronounced at the luminal surface, but which progressively decreased toward the crypt bases (FIG. 4, Table 3). Hyperplastic polyps showed trace to 1+ staining in <10% of luminal epithelial cells. Adenomatous polyps showed only trace diffuse immunostaining. Immunostaining for CTSE was only trace in the cytoplasm of surface epithelial cells in control colon, whereas with both syndromic and sporadic SSA/Ps there was 3 to 4+ staining of the cytoplasm in approximately 75% of epithelial cells that was often more pronounced at the luminal surface but also extended along the crypt axis (FIG. 4, Table 3). Hyperplastic polyps showed only trace to 1+ immunostaining in <25% of epithelial cells. Adenomatous polyps showed only trace staining in rare glands. Immunostaining for TFF2 showed trace to no staining in control colon luminal epithelial cells, whereas SSA/Ps showed 3 to 4+ staining of goblet cell mucin in >60% of both surface and crypt cells (FIG. 4, Table 3). Hyperplastic polyps also showed 2 to 3+ immunostaining of goblet cell mucin in >60% of surface and crypt cells. Adenomatous polyps showed only trace staining in <10% of luminal epithelial cells.

TABLE 3
Immunohistochemical analysis of different serrated and
adenomatous polyp types for proteins encoded by genes
found to be highly differentially expressed in SSA/Ps.

| | VSIG1 | | MUC17 | | CTSE | | TFF2 | |
|---|---|---|---|---|---|---|---|---|
| | | Mean | | Mean | | Mean | | Mean |
| | IHC* | score* | IHC | score | IHC | score | IHC | score |
| Polyp Type | positive | (0-4) | positive | (0-4) | positive | (0-4) | positive | (0-4) |
| Sessile serrated | 11/11* | 3.4 | 12/12 | 2.0 | 11/11 | 3.3 | 10/10 | 3.9 |
| adenoma/polyp, | | | | | | | | |
| syndromic | | | | | | | | |
| Sessile serrated | 23/23 | 3.1 | 17/17 | 2.9 | 15/15 | 2.6 | 15/15 | 3.7 |
| adenoma/polyp, | | | | | | | | |
| sporadic | | | | | | | | |
| Hyperplastic | 5/10 | 1.4 | 3/10 | 0.6 | 3/11 | 1.2 | 11/11 | 2.9 |
| polyp | | | | | | | | |
| Adenomatous | 1/13 | 0.2 | 3/13 | 0.2 | 1/12 | 0.2 | 2/12 | 0.3 |
| polyp | | | | | | | | |
| Uninvolved | 0/8 | 0 | 0/5 | 0 | 0/5 | 0 | 0/4 | 0 |
| colon mucosa | | | | | | | | |
| Normal colon | 0/16 | 0 | 0/11 | 0 | 0/10 | 0 | 0/13 | 0 |
| mucosa | | | | | | | | |

*The number of polyp or normal colonic specimens that showed positive immunohistochemical staining (IHC) over the total number of independent samples examined are shown. IHC staining was scored 0 (none) to 4 (maximal).

In contrast to the other proteins, intense immunostaining for REG4 was found in SSA/Ps, hyperplastic polyps and adenomatous polyps and weak to intermediate staining in control colon (FIG. 6). Specifically, there was 1 to 2+ staining for REG4 in control colonocyte cytoplasm and staining in approximately 50% of goblet cells, whereas with SSA/Ps there was 4+ staining of the full mucosal thickness including 4+ staining of >90% of goblet cells. Hyperplastic polyps also showed 3 to 4+ in >75% of epithelial cells with little staining at the crypt bases. Adenomatous polyps also showed 2 to 3+ immunostaining and in a different (more diffuse pattern) than SSA/Ps or hyperplastic polyps.

### SEQUENCE LISTING

forward primer
SEQ ID NO: 1
5′-AGGGCTCCAGCTTGTATCAC-3′
reverse primer
SEQ ID NO: 2
5′-CGATTCAAGGAGGGTTCTGA-3′
SEQ ID NO: 3 = RefSeq nucleotide sequence encoding human MUC17 (mRNA)
tttcgccagctcctctgggggtgacaggcaagtgagacgtgctcagagctccgatgccaaggcc
agggaccatggcgctgtgtctgctgaccttggtcctctcgctcttgcccccacaagctgctgca
gaacaggacctcagtgtgaacagggctgtgtgggatggaggagggtgcatctcccaaggggacg
tcttgaaccgtcagtgccagcagctgtctcagcacgttaggacaggttctgcggcaaacaccgc
cacaggtacaacatctacaaatgtcgtggagccaagaatgtatttgagttgcagcaccaaccct
gagatgacctcgattgagtccagtgtgacttcagacactcctggtgtctccagtaccaggatga
caccaacagaatccagaacaacttcagaatctaccagtgacagcaccacacttttccccagttc
tactgaagacacttcatctcctacaactcctgaaggcaccgacgtgcccatgtcaacaccaagt
gaagaaagcatttcatcaacaatggcttttgtcagcactgcacctcttcccagttttgaggcct
acacatctttaacatataaggttgatatgagcacacctctgaccacttctactcaggcaagttc
atctcctactactcctgaaagcaccaccatacccaaatcaactaacagtgaaggaagcactcca
ttaacaagtatgcctgccagcaccatgaaggtggccagttcagaggctatcacccttttgacaa
ctcctgttgaaatcagcacacctgtgaccatttctgctcaagccagttcatctcctacaactgc
tgaaggtcccagcctgtcaaactcagctcctagtggaggaagcactccattaacaagaatgcct
ctcagcgtgatgctggtggtcagttctgaggctagcaccctttcaacaactcctgctgccacca
acattcctgtgatcacttctactgaagccagttcatctcctacaacggctgaaggcaccagcat
accaacctcaacttatactgaaggaagcactccattaacaagtacgcctgccagcaccatgccg
gttgccacttctgaaatgagcacactttcaataactcctgttgacaccagcacacttgtgacca
cttctactgaacccagttcacttcctacaactgctgaagctaccagcatgctaacctcaactct
tagtgaaggaagcactccattaacaaatatgcctgtcagcaccatattggtggccagttctgag
gctagcaccacttcaacaattcctgttgactccaaaacttttgtgaccactgctagtgaagcca
gctcatctcccacaactgctgaagataccagcattgcaacctcaactcctagtgaaggaagcac
tccattaacaagtatgcctgtcagcaccactccagtggccagttctgaggctagcaacctttca
acaactcctgttgactccaaaactcaggtgaccacttctactgaagccagttcatctcctccaa
ctgctgaagttaacagcatgccaacctcaactcctagtgaaggaagcactccattaacaagtat
gtctgtcagcaccatgccggtggccagttctgaggctagcaccctttcaacaactcctgttgac
accagcacacctgtgaccacttctagtgaagccagttcatcttctacaactcctgaaggtacca
gcataccaacctcaactcctagtgaaggaagcactccattaacaaacatgcctgtcagcaccag
gctggtggtcagttctgaggctagcaccacttcaacaactcctgctgactccaacacttttgtg
accacttctagtgaagctagttcatcttctacaactgctgaaggtaccagcatgccaacctcaa
cttacagtgaaagaggcactacaataacaagtatgtctgtcagcaccacactggtggccagttc
tgaggctagcaccctttcaacaactcctgttgactccaacactcctgtgaccacttcaactgaa
gccacttcatcttctacaactgcggaaggtaccagcatgccaacctcaacttatactgaaggaa
gcactccattaacaagtatgcctgtcaacaccacactggtggccagttctgaggctagcaccct
ttcaacaactcctgttgacaccagcacacctgtgaccacttcaactgaagccagttcctctcct
acaactgctgatggtgccagtatgccaacctcaactcctagtgaaggaagcactccattaacaa
gtatgcctgtcagcaaaacgctgttgaccagttctgaggctagcaccctttcaacaactcctct
tgacacaagcacacatatcaccacttctactgaagccagttgctctcctacaaccactgaaggt
accagcatgccaatctcaactcctagtgaaggaagtcctttattaacaagtatacctgtcagca
tcacaccggtgaccagtcctgaggctagcaccctttcaacaactcctgttgactccaacagtcc
tgtgaccacttctactgaagtcagttcatctcctacacctgctgaaggtaccagcatgccaacc
tcaacttatagtgaaggaagaactcctttaacaagtatgcctgtcagcaccacactggtggcca
cttctgcaatcagcaccctttcaacaactcctgttgacaccagcacacctgtgaccaattctac
tgaagcccgttcgtctcctacaacttctgaaggtaccagcatgccaacctcaactcctggggaa
ggaagcactccattaacaagtatgcctgacagcaccacgccggtagtcagttctgaggctagaa
cactttcagcaactcctgttgacaccagcacacctgtgaccacttctactgaagccacttcatc
tcctacaactgctgaaggtaccagcataccaacctcgactcctagtgaaggaacgactccatta
acaagcacacctgtcagccacacgctggtggccaattctgaggctagcaccctttcaacaactc
ctgttgactccaacactcctttgaccacttctactgaagccagttcacctcctcccactgctga
aggtaccagcatgccaacctcaactcctagtgaaggaagcactccattaacacgtatgcctgtc
agcaccacaatggtggccagttctgaaacgagcacactttcaacaactcctgctgacaccagca
cacctgtgaccacttattctcaagccagttcatcttctacaactgctgacggtaccagcatgcc
aacctcaacttatagtgaaggaagcactccactaacaagtgtgcctgtcagcaccaggctggtg
gtcagttctgaggctagcaccctttccacaactcctgtcgacaccagcatacctgtcaccactt
ctactgaagccagttcatctcctacaactgctgaaggtaccagcataccaacctcacctcccag
tgaaggaaccactccgttagcaagtatgcctgtcagcaccacgctggtggtcagttctgaggct
aacaccctttcaacaactcctgtggactccaaaactcaggtggccacttctactgaagccagtt
cacctcctccaactgctgaagttaccagcatgccaacctcaactcctggagaaagaagcactcc
attaacaagtatgcctgtcagacacacgccagtggccagttctgaggctagcaccctttcaaca
tctcccgttgacaccagcacacctgtgaccacttctgctgaaaccagttcctctcctacaaccg
ctgaaggtaccagcttgccaacctcaactactagtgaaggaagtactctattaacaagtatacc
tgtcagcaccacgctggtgaccagtcctgaggctagcacccttttaacaactcctgttgacact
aaaggtcctgtggtcacttctaatgaagtcagttcatctcctacacctgctgaaggtaccagca
tgccaacctcaacttatagtgaaggaagaactcctttaacaagtatacctgtcaacaccacact
ggtggccagttctgcaatcagcatcctttcaacaactcctgttgacaacagcacacctgtgacc
acttctactgaagcctgttcatctcctacaacttctgaaggtaccagcatgccaaactcaaatc
ctagtgaaggaaccactccgttaacaagtatacctgtcagcaccacgccggtagtcagttctga
ggctagcaccctttcagcaactcctgttgacaccagcacccctgggaccacttctgctgaagcc
acttcatctcctacaactgctgaaggtatcagcataccaacctcaactcctagtgaaggaaaga
ctccattaaaaagtatacctgtcagcaacacgccggtggccaattctgaggctagcaccctttc
aacaactcctgttgactctaacagtcctgtggtcacttctacagcagtcagttcatctcctaca
cctgctgaaggtaccagcatagcaatctcaacgcctagtgaaggaagcactgcattaacaagta
tacctgtcagcaccacaacagtggccagttctgaaatcaacagcctttcaacaactcctgctgt
caccagcacacctgtgaccacttattctcaagccagttcatctcctacaactgctgacggtacc
agcatgcaaacctcaacttatagtgaaggaagcactccactaacaagtttgcctgtcagcacca
tgctggtggtcagttctgaggctaacaccctttcaacaacccctattgactccaaaactcaggt
gaccgcttctactgaagccagttcatctacaaccgctgaaggtagcagcatgacaatctcaact
cctagtgaaggaagtcctctattaacaagtatacctgtcagcaccacgccggtggccagtcctg
aggctagcaccctttcaacaactcctgttgactccaacagtcctgtgatcacttctactgaagt
cagttcatctcctacacctgctgaaggtaccagcatgccaacctcaacttatactgaaggaaga
actcctttaacaagtataactgtcagaacaacaccggtggccagctctgcaatcagcacccttt
caacaactcccgttgacaacagcacacctgtgaccacttctactgaagcccgttcatctcctac
aacttctgaaggtaccagcatgccaaactcaactcctagtgaaggaaccactccattaacaagt
atacctgtcagcaccacgccggtactcagttctgaggctagcaccctttcagcaactcctattg
acaccagcacccctgtgaccacttctactgaagccacttcgtctcctacaactgctgaaggtac
cagcataccaacctcgactcttagtgaaggaatgactccattaacaagcacacctgtcagccac
acgctggtggccaattctgaggctagcaccctttcaacaactcctgttgactctaacagtcctg
tggtcacttctacagcagtcagttcatctcctacacctgctgaaggtaccagcatagcaacctc
aacgcctagtgaaggaagcactgcattaacaagtatacctgtcagcaccacaacagtggccagt
tctgaaaccaacaccctttcaacaactcccgctgtcaccagcacacctgtgaccacttatgctc
aagtcagttcatctcctacaactgctgacggtagcagcatgccaacctcaactcctagggaagg
aaggcctccattaacaagtatacctgtcagcaccacaacagtggccagttctgaaatcaacacc
ctttcaacaactcttgctgacaccaggacacctgtgaccacttattctcaagccagttcatctc
ctacaactgctgatggtaccagcatgccaaccccagcttatagtgaaggaagcactccactaac
aagtatgcctctcagcaccacgctggtggtcagttctgaggctagcactctttccacaactcct
gttgacaccagcactcctgccaccacttctactgaaggcagttcatctcctacaactgcaggag
gtaccagcatacaaacctcaactcctagtgaacggaccactccattagcaggtatgcctgtcag
cactacgcttgtggtcagttctgagggtaacaccctttcaacaactcctgttgactccaaaact
caggtgaccaattctactgaagccagttcatctgcaaccgctgaaggtagcagcatgacaatct
cagctcctagtgaaggaagtcctctactaacaagtatacctctcagcaccacgccggtggccag
tcctgaggctagcaccctttcaacaactcctgttgactccaacagtcctgtgatcacttctact
gaagtcagttcatctcctatacctactgaaggtaccagcatgcaaacctcaacttatagtgaca
gaagaactcctttaacaagtatgcctgtcagcaccacagtggtggccagttctgcaatcagcac
cctttcaacaactcctgttgacaccagcacacctgtgaccaattctactgaagcccgttcatct
cctacaacttctgaaggtaccagcatgccaacctcaactcctagtgaaggaagcactccattca
caagtatgcctgtcagcaccatgccggtagttacttctgaggctagcaccctttcagcaactcc
tgttgacaccagcacacctgtgaccacttctactgaagccacttcatctcctacaactgctgaa
ggtaccagcataccaacttcaactcttagtgaaggaacgactccattaacaagtatacctgtca
gccacacgctggtggccaattctgaggttagcaccctttcaacaactcctgttgactccaacac
tcctttcactacttctactgaagccagttcacctcctcccactgctgaaggtaccagcatgcca
acctcaacttctagtgaaggaaacactccattaacacgtatgcctgtcagcaccacaatggtgg
ccagttttgaaacaagcacactttctacaactcctgctgacaccagcacacctgtgactactta
ttctcaagccggttcatctcctacaactgctgacgatactagcatgccaacctcaacttatagt
gaaggaagcactccactaacaagtgtgcctgtcagcaccatgccggtggtcagttctgaggcta
gcacccattccacaactcctgttgacaccagcacacctgtcaccacttctactgaagccagttc
atctcctacaactgctgaaggtaccagcataccaacctcacctcctagtgaaggaaccactccg
ttagcaagtatgcctgtcagcaccacgccggtggtcagttctgaggctggcaccctttccacaa
ctcctgttgacaccagcacacctatgaccacttctactgaagccagttcatctcctacaactgc
tgaagatatcgtcgtgccaatctcaactgctagtgaaggaagtactctattaacaagtatacct
gtcagcaccacgccagtggccagtcctgaggctagcaccctttcaacaactcctgttgactcca
acagtcctgtggtcacttctactgaaatcagttcatctgctacatccgctgaaggtaccagcat
gcctacctcaacttatagtgaaggaagcactccattaagaagtatgcctgtcagcaccaagccg
ttggccagttctgaggctagcactctttcaacaactcctgttgacaccagcatacctgtcacca
cttctactgaaaccagttcatctcctacaactgcaaaagataccagcatgccaatctcaactcc
tagtgaagtaagtacttcattaacaagtatacttgtcagcaccatgccagtggccagttctgag
gctagcaccctttcaacaactcctgttgacaccaggacacttgtgaccacttccactggaacca
gttcatctcctacaactgctgaaggtagcagcatgccaacctcaactcctggtgaaagaagcac
tccattaacaaatatacttgtcagcaccacgctgttggccaattctgaggctagcaccctttca
acaactcctgttgacaccagcacacctgtcaccacttctgctgaagccagttcttctcctacaa
ctgctgaaggtaccagcatgcgaatctcaactcctagtgatggaagtactccattaacaagtat
acttgtcagcaccctgccagtggccagttctgaggctagcaccgtttcaacaactgctgttgac
accagcatacctgtcaccacttctactgaagccagttcctctcctacaactgctgaagttacca
gcatgccaacctcaactcctagtgaaacaagtactccattaactagtatgcctgtcaaccacac
gccagtggccagttctgaggctggcaccctttcaacaactcctgttgacaccagcacacctgtg
accacttctactaaagccagttcatctcctacaactgctgaaggtatcgtcgtgccaatctcaa
ctgctagtgaaggaagtactctattaacaagtatacctgtcagcaccacgccggtggccagttc
tgaggctagcaccctttcaacaactcctgttgataccagcatacctgtcaccacttctactgaa
ggcagttcttctcctacaactgctgaaggtaccagcatgccaatctcaactcctagtgaagtaa
gtactccattaacaagtatacttgtcagcaccgtgccagtggccggttctgaggctagcaccct
ttcaacaactcctgttgacaccaggacacctgtcaccacttctgctgaagctagttcttctcct
acaactgctgaaggtaccagcatgccaatctcaactcctggcgaaagaagaactccattaacaa
gtatgtctgtcagcaccatgccggtggccagttctgaggctagcaccctttcaagaactcctgc
tgacaccagcacacctgtgaccacttctactgaagccagttcctctcctacaactgctgaaggt
accggcataccaatctcaactcctagtgaaggaagtactccattaacaagtatacctgtcagca
ccacgccagtggccattcctgaggctagcaccctttcaacaactcctgttgactccaacagtcc
tgtggtcacttctactgaagtcagttcatctcctacacctgctgaaggtaccagcatgccaatc
tcaacttatagtgaaggaagcactccattaacaggtgtgcctgtcagcaccacaccggtgacca
gttctgcaatcagcaccctttcaacaactcctgttgacaccagcacacctgtgaccacttctac
tgaagcccattcatctcctacaacttctgaaggtaccagcatgccaacctcaactcctagtgaa
ggaagtactccattaacatatatgcctgtcagcaccatgctggtagtcagttctgaggatagca
ccctttcagcaactcctgttgacaccagcacacctgtgaccacttctactgaagccacttcatc
tacaactgctgaaggtaccagcattccaacctcaactcctagtgaaggaatgactccattaact
agtgtacctgtcagcaacacgccggtggccagttctgaggctagcatcctttcaacaactcctg
ttgactccaacactcctttgaccacttctactgaagccagttcatctcctcccactgctgaagg
taccagcatgccaacctcaactcctagtgaaggaagcactccattaacaagtatgcctgtcagc
accacaacggtggccagttctgaaacgagcaccctttcaacaactcctgctgacaccagcacac
ctgtgaccacttattctcaagccagttcatctcctccaattgctgacggtactagcatgccaac
ctcaacttatagtgaaggaagcactccactaacaaatatgtctttcagcaccacgccagtggtc
agttctgaggctagcaccctttccacaactcctgttgacaccagcacacctgtcaccacttcta
ctgaagccagtttatctcctacaactgctgaaggtaccagcataccaacctcaagtcctagtga
aggaaccactccattagcaagtatgcctgtcagcaccacgccggtggtcagttctgaggttaac
accctttcaacaactcctgtggactccaacactctggtgaccacttctactgaagccagttcat
ctcctacaatcgctgaaggtaccagcttgccaacctcaactactagtgaaggaagcactccatt
atcaattatgcctctcagtaccacgccggtggccagttctgaggctagcaccctttcaacaact
cctgttgacaccagcacacctgtgaccacttcttctccaaccaattcatctcctacaactgctg
aagttaccagcatgccaacatcaactgctggtgaaggaagcactccattaacaaatatgcctgt
cagcaccacaccggtggccagttctgaggctagcaccctttcaacaactcctgttgactccaac
acttttgttaccagttctagtcaagccagttcatctccagcaactcttcaggtcaccactatgc
gtatgtctactccaagtgaaggaagctcttcattaacaactatgctcctcagcagcacatatgt
gaccagttctgaggctagcacaccttccactccttctgttgacagaagcacacctgtgaccact
tctactcagagcaattctactcctacacctcctgaagttatcaccctgccaatgtcaactccta
gtgaagtaagcactccattaaccattatgcctgtcagcaccacatcggtgaccatttctgaggc
tggcacagcttcaacacttcctgttgacaccagcacacctgtgatcacttctacccaagtcagt
tcatctcctgtgactcctgaaggtaccaccatgccaatctggacgcctagtgaaggaagcactc
cattaacaactatgcctgtcagcaccacacgtgtgaccagctctgagggtagcaccctttcaac
accttctgttgtcaccagcacacctgtgaccacttctactgaagccatttcatcttctgcaact
cttgacagcaccaccatgtctgtgtcaatgcccatggaaataagcacccttgggaccactattc
ttgtcagtaccacacctgttacgaggtttcctgagagtagcaccccttccataccatctgttta
caccagcatgtctatgaccactgcctctgaaggcagttcatctcctacaactcttgaaggcacc
accaccatgcctatgtcaactacgagtgaaagaagcactttattgacaactgtcctcatcagcc
ctatatctgtgatgagtccttctgaggccagcacactttcaacacctcctggtgataccagcac
acctttgctcacctctaccaaagccggttcattctccatacctgctgaagtcactaccatacgt
atttcaattaccagtgaaagaagcactccattaacaactctccttgtcagcaccacacttccaa
ctagctttcctggggccagcatagcttcgacacctcctcttgacacaagcacaacttttacccc
ttctactgacactgcctcaactcccacaattcctgtagccaccaccatatctgtatcagtgatc
acagaaggaagcacacctgggacaaccatttttattcccagcactcctgtcaccagttctactg
ctgatgtctttcctgcaacaactggtgctgtatctacccctgtgataacttccactgaactaaa
cacaccatcaacctccagtagtagtaccaccacatctttttcaactactaaggaatttacaaca
cccgcaatgactactgcagctcccctcacatatgtgaccatgtctactgcccccagcacaccca
gaacaaccagcagaggctgcactacttctgcatcaacgctttctgcaaccagtacacctcacac
ctctacttctgtcaccacccgtcctgtgaccccttcatcagaatccagcaggccgtcaacaatt
acttctcacaccatcccacctacatttcctcctgctcactccagtacacctccaacaacctctg
cctcctccacgactgtgaaccctgaggctgtcaccaccatgaccaccaggacaaaacccagcac
acggaccacttccttccccacggtgaccaccaccgctgtccccacgaatactacaattaagagc
aaccccacctcaactcctactgtgccaagaaccacaacatgctttggagatgggtgccagaata
cggcctctcgctgcaagaatggaggcacctgggatgggctcaagtgccagtgtcccaacctcta
ttatggggagttgtgtgaggaggtggtcagcagcattgacatagggccaccggagactatctct
gcccaaatggaactgactgtgacagtgaccagtgtgaagttcaccgaagagctaaaaaaccact
cttcccaggaattccaggagttcaaacagacattcacggaacagatgaatattgtgtattccgg
gatccctgagtatgtcggggtgaacatcacaaagctacgtcttggcagtgtggtggtggagcat
gacgtcctcctaagaaccaagtacacaccagaatacaagacagtattggacaatgccaccgaag
tagtgaaagagaaaatcacaaaagtgaccacacagcaaataatgattaatgatatttgctcaga
catgatgtgtttcaacaccactggcacccaagtgcaaaacattacggtgacccagtacgaccct
gaagaggactgccggaagatggccaaggaatatggagactacttcgtagtggagtaccgggacc
agaagccatactgcatcagcccctgtgagcctggcttcagtgtctccaagaactgtaacctcgg
caagtgccagatgtctctaagtggacctcagtgcctctgcgtgaccacggaaactcactggtac
agtggggagacctgtaaccagggcacccagaagagtctggtgtacggcctcgtgggggcagggg
tcgtgctgatgctgatcatcctggtagctctcctgatgctcgttttccgctccaagagagaggt
gaaacggcaaaagtacagattgtctcagttatacaagtggcaagaagaggacagtggaccagct
cctgggaccttccaaaacattggctttgacatctgccaagatgatgattccatccacctggagt
ccatctatagtaatttccagccctccttgagacacatagaccctgaaacaaagatccgaattca
gaggcctcaggtaatgacgacatcattttaaggcatggagctgagaagtctgggagtgaggaga
tcccagtccggctaagcttggtggagcattttcccattgagagccttccatgggaactcaatgt
tcccattgtaagtacaggaaacaagccctgtacttaccaaggagaaagaggagagacagcagtg
ctgggagattctcaaatagaaacccgtggacgctccaatgggcttgtcatgatatcaggctagg
ctttcctgctcatttttcaaagacgctccagatttgagggtactctgactgcaacatctttcac
cccattgatcgccaggattgatttggttgatctggctgagcaggcgggtgtccccgtcctccct
cactgccccatatgtgtccctcctaaagctgcatgctcagttgaagaggacgagaggacgacct
tctctgatagaggaggaccacgcttcagtcaaaggcatacaagtatctatctggacttccctgc
tagcacttccaaacaagctcagagatgttcctcccctcatctgcccgggttcagtaccatggac
agcgccctcgacccgctgtttacaaccatgaccccttggacactggactgcatgcactttacat
atcacaaaatgctctcataagaattattgcataccatcttcatgaaaaacacctgtatttaaat
atagagcatttaccttttggtatataagattgtgggtattttttaagttcttattgttatgagt
tctgattttttccttagtaaatattataatatatatttgtagtaactaaaaataataaagcaat
tttattacaattttaaaaaaaaaa
SEQ ID NO: 4 = RefSeq polypeptide sequence of human MUC17
(4493 amino acids)
MPRPGTMALCLLTLVLSLLPPQAAAEQDLSVNRAVWDGGGCISQGDVLNRQCQQLSQHVRTGSA
ANTATGTTSTNVVEPRMYLSCSTNPEMTSIESSVTSDTPGVSSTRMTPTESRTTSESTSDSTTL
FPSSTEDTSSPTTPEGTDVPMSTPSEESISSTMAFVSTAPLPSFEAYTSLTYKVDMSTPLTTST
QASSSPTTPESTTIPKSTNSEGSTPLTSMPASTMKVASSEAITLLTTPVEISTPVTISAQASSS
PTTAEGPSLSNSAPSGGSTPLTRMPLSVMLVVSSEASTLSTTPAATNIPVITSTEASSSPTTAE
GTSIPTSTYTEGSTPLTSTPASTMPVATSEMSTLSITPVDTSTLVTTSTEPSSLPTTAEATSML
TSTLSEGSTPLTNMPVSTILVASSEASTTSTIPVDSKTFVTTASEASSSPTTAEDTSIATSTPS
EGSTPLTSMPVSTTPVASSEASNLSTTPVDSKTQVTTSTEASSSPPTAEVNSMPTSTPSEGSTP
LTSMSVSTMPVASSEASTLSTTPVDTSTPVTTSSEASSSSTTPEGTSIPTSTPSEGSTPLTNMP
VSTRLVVSSEASTTSTTPADSNTFVTTSSEASSSSTTAEGTSMPTSTYSERGTTITSMSVSTTL
VASSEASTLSTTPVDSNTPVTTSTEATSSSTTAEGTSMPTSTYTEGSTPLTSMPVNTTLVASSE
ASTLSTTPVDTSTPVTTSTEASSSPTTADGASMPTSTPSEGSTPLTSMPVSKTLLTSSEASTLS
TTPLDTSTHITTSTEASCSPTTTEGTSMPISTPSEGSPLLTSIPVSITPVTSPEASTLSTTPVD
SNSPVTTSTEVSSSPTPAEGTSMPTSTYSEGRTPLTSMPVSTTLVATSAISTLSTTPVDTSTPV
TNSTEARSSPTTSEGTSMPTSTPGEGSTPLTSMPDSTTPVVSSEARTLSATPVDTSTPVTTSTE
ATSSPTTAEGTSIPTSTPSEGTTPLTSTPVSHTLVANSEASTLSTTPVDSNTPLTTSTEASSPP
PTAEGTSMPTSTPSEGSTPLTRMPVSTTMVASSETSTLSTTPADTSTPVTTYSQASSSSTTADG
TSMPTSTYSEGSTPLTSVPVSTRLVVSSEASTLSTTPVDTSIPVTTSTEASSSPTTAEGTSIPT
SPPSEGTTPLASMPVSTTLVVSSEANTLSTTPVDSKTQVATSTEASSPPPTAEVTSMPTSTPGE
RSTPLTSMPVRHTPVASSEASTLSTSPVDTSTPVTTSAETSSSPTTAEGTSLPTSTTSEGSTLL
TSIPVSTTLVTSPEASTLLTTPVDTKGPVVTSNEVSSSPTPAEGTSMPTSTYSEGRTPLTSIPV
NTTLVASSAISILSTTPVDNSTPVTTSTEACSSPTTSEGTSMPNSNPSEGTTPLTSIPVSTTPV
VSSEASTLSATPVDTSTPGTTSAEATSSPTTAEGISIPTSTPSEGKTPLKSIPVSNTPVANSEA
STLSTTPVDSNSPVVTSTAVSSSPTPAEGTSIAISTPSEGSTALTSIPVSTTTVASSEINSLST
TPAVTSTPVTTYSQASSSPTTADGTSMQTSTYSEGSTPLTSLPVSTMLVVSSEANTLSTTPIDS
KTQVTASTEASSSTTAEGSSMTISTPSEGSPLLTSIPVSTTPVASPEASTLSTTPVDSNSPVIT
STEVSSSPTPAEGTSMPTSTYTEGRTPLTSITVRTTPVASSAISTLSTTPVDNSTPVTTSTEAR
SSPTTSEGTSMPNSTPSEGTTPLTSIPVSTTPVLSSEASTLSATPIDTSTPVTTSTEATSSPTT
AEGTSIPTSTLSEGMTPLTSTPVSHTLVANSEASTLSTTPVDSNSPVVTSTAVSSSPTPAEGTS
IATSTPSEGSTALTSIPVSTTTVASSETNTLSTTPAVTSTPVTTYAQVSSSPTTADGSSMPTST
PREGRPPLTSIPVSTTTVASSEINTLSTTLADTRTPVTTYSQASSSPTTADGTSMPTPAYSEGS
TPLTSMPLSTTLVVSSEASTLSTTPVDTSTPATTSTEGSSSPTTAGGTSIQTSTPSERTTPLAG
MPVSTTLVVSSEGNTLSTTPVDSKTQVTNSTEASSSATAEGSSMTISAPSEGSPLLTSIPLSTT
PVASPEASTLSTTPVDSNSPVITSTEVSSSPIPTEGTSMQTSTYSDRRTPLTSMPVSTTVVASS
AISTLSTTPVDTSTPVTNSTEARSSPTTSEGTSMPTSTPSEGSTPFTSMPVSTMPVVTSEASTL
SATPVDTSTPVTTSTEATSSPTTAEGTSIPTSTLSEGTTPLTSIPVSHTLVANSEVSTLSTTPV
DSNTPFTTSTEASSPPPTAEGTSMPTSTSSEGNTPLTRMPVSTTMVASFETSTLSTTPADTSTP
VTTYSQAGSSPTTADDTSMPTSTYSEGSTPLTSVPVSTMPVVSSEASTHSTTPVDTSTPVTTST
EASSSPTTAEGTSIPTSPPSEGTTPLASMPVSTTPVVSSEAGTLSTTPVDTSTPMTTSTEASSS
PTTAEDIVVPISTASEGSTLLTSIPVSTTPVASPEASTLSTTPVDSNSPVVTSTEISSSATSAE
GTSMPTSTYSEGSTPLRSMPVSTKPLASSEASTLSTTPVDTSIPVTTSTETSSSPTTAKDTSMP
ISTPSEVSTSLTSILVSTMPVASSEASTLSTTPVDTRTLVTTSTGTSSSPTTAEGSSMPTSTPG
ERSTPLTNILVSTTLLANSEASTLSTTPVDTSTPVTTSAEASSSPTTAEGTSMRISTPSDGSTP
LTSILVSTLPVASSEASTVSTTAVDTSIPVTTSTEASSSPTTAEVTSMPTSTPSETSTPLTSMP
VNHTPVASSEAGTLSTTPVDTSTPVTTSTKASSSPTTAEGIVVPISTASEGSTLLTSIPVSTTP
VASSEASTLSTTPVDTSIPVTTSTEGSSSPTTAEGTSMPISTPSEVSTPLTSILVSTVPVAGSE
ASTLSTTPVDTRTPVTTSAEASSSPTTAEGTSMPISTPGERRTPLTSMSVSTMPVASSEASTLS
RTPADTSTPVTTSTEASSSPTTAEGTGIPISTPSEGSTPLTSIPVSTTPVAIPEASTLSTTPVD
SNSPVVTSTEVSSSPTPAEGTSMPISTYSEGSTPLTGVPVSTTPVTSSAISTLSTTPVDTSTPV
TTSTEAHSSPTTSEGTSMPTSTPSEGSTPLTYMPVSTMLVVSSEDSTLSATPVDTSTPVTTSTE
ATSSTTAEGTSIPTSTPSEGMTPLTSVPVSNTPVASSEASILSTTPVDSNTPLTTSTEASSSPP
TAEGTSMPTSTPSEGSTPLTSMPVSTTTVASSETSTLSTTPADTSTPVTTYSQASSSPPIADGT
SMPTSTYSEGSTPLTNMSFSTTPVVSSEASTLSTTPVDTSTPVTTSTEASLSPTTAEGTSIPTS
SPSEGTTPLASMPVSTTPVVSSEVNTLSTTPVDSNTLVTTSTEASSSPTIAEGTSLPTSTTSEG
STPLSIMPLSTTPVASSEASTLSTTPVDTSTPVTTSSPTNSSPTTAEVTSMPTSTAGEGSTPLT
NMPVSTTPVASSEASTLSTTPVDSNTFVTSSSQASSSPATLQVTTMRMSTPSEGSSSLTTMLLS
STYVTSSEASTPSTPSVDRSTPVTTSTQSNSTPTPPEVITLPMSTPSEVSTPLTIMPVSTTSVT
ISEAGTASTLPVDTSTPVITSTQVSSSPVTPEGTTMPIWTPSEGSTPLTTMPVSTTRVTSSEGS
TLSTPSVVTSTPVTTSTEAISSSATLDSTTMSVSMPMEISTLGTTILVSTTPVTRFPESSTPSI
PSVYTSMSMTTASEGSSSPTTLEGTTTMPMSTTSERSTLLTTVLISPISVMSPSEASTLSTPPG
DTSTPLLTSTKAGSFSIPAEVTTIRISITSERSTPLTTLLVSTTLPTSFPGASIASTPPLDTST
TFTPSTDTASTPTIPVATTISVSVITEGSTPGTTIFIPSTPVTSSTADVFPATTGAVSTPVITS
TELNTPSTSSSSTTTSFSTTKEFTTPAMTTAAPLTYVTMSTAPSTPRTTSRGCTTSASTLSATS
TPHTSTSVTTRPVTPSSESSRPSTITSHTIPPTFPPAHSSTPPTTSASSTTVNPEAVTTMTTRT
KPSTRTTSFPTVTTTAVPTNTTIKSNPTSTPTVPRTTTCFGDGCQNTASRCKNGGTWDGLKCQC
PNLYYGELCEEVVSSIDIGPPETISAQMELTVTVTSVKFTEELKNHSSQEFQEFKQTFTEQMNI
VYSGIPEYVGVNITKLRLGSVVVEHDVLLRTKYTPEYKTVLDNATEVVKEKITKVTTQQIMIND
ICSDMMCFNTTGTQVQNITVTQYDPEEDCRKMAKEYGDYFVVEYRDQKPYCISPCEPGFSVSKN
CNLGKCQMSLSGPQCLCVTTETHWYSGETCNQGTQKSLVYGLVGAGVVLMLIILVALLMLVFRS
KREVKRQKYRLSQLYKWQEEDSGPAPGTFQNIGFDICQDDDSIHLESIYSNFQPSLRHIDPETK
IRIQRPQVMTTSF
SEQ ID NO: 5 = Ensembl nucleotide sequence encoding human MUC17 (mRNA)
tctgaggctcatttcgccagctcctctgggggtgacaggcaagtgagacgtgctcagagctccg
ATGCCAAGGCCAGGGACCATGGCGCTGTGTCTGCTGACCTTGGTCCTCTCGCTCTTGCCCCCAC
AAGCTGCTGCAGAACAGGACCTCAGTGTGAACAGGGCTGTGTGGGATGGAGGAGGGTGCATCTC
CCAAGGGGACGTCTTGAACCGTCAGTGCCAGCAGCTGTCTCAGCACGTTAGGACAGGTTCTGCG
GCAAACACCGCCACAGGTACAACATCTACAAATGTCGTGGAGCCAAGAATGTATTTGAGTTGCA
GCACCAACCCTGAGATGACCTCGATTGAGTCCAGTGTGACTTCAGACACTCCTGGTGTCTCCAG
TACCAGGATGACACCAACAGAATCCAGAACAACTTCAGAATCTACCAGTGACAGCACCACACTT
TTCCCCAGTTCTACTGAAGACACTTCATCTCCTACAACTCCTGAAGGCACCGACGTGCCCATGT
CAACACCAAGTGAAGAAAGCATTTCATCAACAATGGCTTTTGTCAGCACTGCACCTCTTCCCAG
TTTTGAGGCCTACACATCTTTAACATATAAGGTTGATATGAGCACACCTCTGACCACTTCTACT
CAGGCAAGTTCATCTCCTACTACTCCTGAAAGCACCACCATACCCAAATCAACTAACAGTGAAG
GAAGCACTCCATTAACAAGTATGCCTGCCAGCACCATGAAGGTGGCCAGTTCAGAGGCTATCAC
CCTTTTGACAACTCCTGTTGAAATCAGCACACCTGTGACCATTTCTGCTCAAGCCAGTTCATCT
CCTACAACTGCTGAAGGTCCCAGCCTGTCAAACTCAGCTCCTAGTGGAGGAAGCACTCCATTAA
CAAGAATGCCTCTCAGCGTGATGCTGGTGGTCAGTTCTGAGGCTAGCACCCTTTCAACAACTCC
TGCTGCCACCAACATTCCTGTGATCACTTCTACTGAAGCCAGTTCATCTCCTACAACGGCTGAA
GGCACCAGCATACCAACCTCAACTTATACTGAAGGAAGCACTCCATTAACAAGTACGCCTGCCA
GCACCATGCCGGTTGCCACTTCTGAAATGAGCACACTTTCAATAACTCCTGTTGACACCAGCAC
ACTTGTGACCACTTCTACTGAACCCAGTTCACTTCCTACAACTGCTGAAGCTACCAGCATGCTA
ACCTCAACTCTTAGTGAAGGAAGCACTCCATTAACAAATATGCCTGTCAGCACCATATTGGTGG
CCAGTTCTGAGGCTAGCACCACTTCAACAATTCCTGTTGACTCCAAAACTTTTGTGACCACTGC
TAGTGAAGCCAGCTCATCTCCCACAACTGCTGAAGATACCAGCATTGCAACCTCAACTCCTAGT
GAAGGAAGCACTCCATTAACAAGTATGCCTGTCAGCACCACTCCAGTGGCCAGTTCTGAGGCTA
GCAACCTTTCAACAACTCCTGTTGACTCCAAAACTCAGGTGACCACTTCTACTGAAGCCAGTTC
ATCTCCTCCAACTGCTGAAGTTAACAGCATGCCAACCTCAACTCCTAGTGAAGGAAGCACTCCA
TTAACAAGTATGTCTGTCAGCACCATGCCGGTGGCCAGTTCTGAGGCTAGCACCCTTTCAACAA
CTCCTGTTGACACCAGCACACCTGTGACCACTTCTAGTGAAGCCAGTTCATCTTCTACAACTCC
TGAAGGTACCAGCATACCAACCTCAACTCCTAGTGAAGGAAGCACTCCATTAACAAACATGCCT
GTCAGCACCAGGCTGGTGGTCAGTTCTGAGGCTAGCACCACTTCAACAACTCCTGCTGACTCCA
ACACTTTTGTGACCACTTCTAGTGAAGCTAGTTCATCTTCTACAACTGCTGAAGGTACCAGCAT
GCCAACCTCAACTTACAGTGAAAGAGGCACTACAATAACAAGTATGTCTGTCAGCACCACACTG
GTGGCCAGTTCTGAGGCTAGCACCCTTTCAACAACTCCTGTTGACTCCAACACTCCTGTGACCA
CTTCAACTGAAGCCACTTCATCTTCTACAACTGCGGAAGGTACCAGCATGCCAACCTCAACTTA
TACTGAAGGAAGCACTCCATTAACAAGTATGCCTGTCAACACCACACTGGTGGCCAGTTCTGAG
GCTAGCACCCTTTCAACAACTCCTGTTGACACCAGCACACCTGTGACCACTTCAACTGAAGCCA
GTTCCTCTCCTACAACTGCTGATGGTGCCAGTATGCCAACCTCAACTCCTAGTGAAGGAAGCAC
TCCATTAACAAGTATGCCTGTCAGCAAAACGCTGTTGACCAGTTCTGAGGCTAGCACCCTTTCA
ACAACTCCTCTTGACACAAGCACACATATCACCACTTCTACTGAAGCCAGTTGCTCTCCTACAA
CCACTGAAGGTACCAGCATGCCAATCTCAACTCCTAGTGAAGGAAGTCCTTTATTAACAAGTAT
ACCTGTCAGCATCACACCGGTGACCAGTCCTGAGGCTAGCACCCTTTCAACAACTCCTGTTGAC
TCCAACAGTCCTGTGACCACTTCTACTGAAGTCAGTTCATCTCCTACACCTGCTGAAGGTACCA
GCATGCCAACCTCAACTTATAGTGAAGGAAGAACTCCTTTAACAAGTATGCCTGTCAGCACCAC
ACTGGTGGCCACTTCTGCAATCAGCACCCTTTCAACAACTCCTGTTGACACCAGCACACCTGTG
ACCAATTCTACTGAAGCCCGTTCGTCTCCTACAACTTCTGAAGGTACCAGCATGCCAACCTCAA
CTCCTGGGGAAGGAAGCACTCCATTAACAAGTATGCCTGACAGCACCACGCCGGTAGTCAGTTC
TGAGGCTAGAACACTTTCAGCAACTCCTGTTGACACCAGCACACCTGTGACCACTTCTACTGAA
GCCACTTCATCTCCTACAACTGCTGAAGGTACCAGCATACCAACCTCGACTCCTAGTGAAGGAA
CGACTCCATTAACAAGCACACCTGTCAGCCACACGCTGGTGGCCAATTCTGAGGCTAGCACCCT
TTCAACAACTCCTGTTGACTCCAACACTCCTTTGACCACTTCTACTGAAGCCAGTTCACCTCCT
CCCACTGCTGAAGGTACCAGCATGCCAACCTCAACTCCTAGTGAAGGAAGCACTCCATTAACAC
GTATGCCTGTCAGCACCACAATGGTGGCCAGTTCTGAAACGAGCACACTTTCAACAACTCCTGC
TGACACCAGCACACCTGTGACCACTTATTCTCAAGCCAGTTCATCTTCTACAACTGCTGACGGT
ACCAGCATGCCAACCTCAACTTATAGTGAAGGAAGCACTCCACTAACAAGTGTGCCTGTCAGCA
CCAGGCTGGTGGTCAGTTCTGAGGCTAGCACCCTTTCCACAACTCCTGTCGACACCAGCATACC
TGTCACCACTTCTACTGAAGCCAGTTCATCTCCTACAACTGCTGAAGGTACCAGCATACCAACC
TCACCTCCCAGTGAAGGAACCACTCCGTTAGCAAGTATGCCTGTCAGCACCACGCTGGTGGTCA
GTTCTGAGGCTAACACCCTTTCAACAACTCCTGTGGACTCCAAAACTCAGGTGGCCACTTCTAC
TGAAGCCAGTTCACCTCCTCCAACTGCTGAAGTTACCAGCATGCCAACCTCAACTCCTGGAGAA
AGAAGCACTCCATTAACAAGTATGCCTGTCAGACACACGCCAGTGGCCAGTTCTGAGGCTAGCA
CCCTTTCAACATCTCCCGTTGACACCAGCACACCTGTGACCACTTCTGCTGAAACCAGTTCCTC
TCCTACAACCGCTGAAGGTACCAGCTTGCCAACCTCAACTACTAGTGAAGGAAGTACTCTATTA
ACAAGTATACCTGTCAGCACCACGCTGGTGACCAGTCCTGAGGCTAGCACCCTTTTAACAACTC
CTGTTGACACTAAAGGTCCTGTGGTCACTTCTAATGAAGTCAGTTCATCTCCTACACCTGCTGA
AGGTACCAGCATGCCAACCTCAACTTATAGTGAAGGAAGAACTCCTTTAACAAGTATACCTGTC
AACACCACACTGGTGGCCAGTTCTGCAATCAGCATCCTTTCAACAACTCCTGTTGACAACAGCA
CACCTGTGACCACTTCTACTGAAGCCTGTTCATCTCCTACAACTTCTGAAGGTACCAGCATGCC
AAACTCAAATCCTAGTGAAGGAACCACTCCGTTAACAAGTATACCTGTCAGCACCACGCCGGTA
GTCAGTTCTGAGGCTAGCACCCTTTCAGCAACTCCTGTTGACACCAGCACCCCTGGGACCACTT
CTGCTGAAGCCACTTCATCTCCTACAACTGCTGAAGGTATCAGCATACCAACCTCAACTCCTAG
TGAAGGAAAGACTCCATTAAAAAGTATACCTGTCAGCAACACGCCGGTGGCCAATTCTGAGGCT
AGCACCCTTTCAACAACTCCTGTTGACTCTAACAGTCCTGTGGTCACTTCTACAGCAGTCAGTT
CATCTCCTACACCTGCTGAAGGTACCAGCATAGCAATCTCAACGCCTAGTGAAGGAAGCACTGC
ATTAACAAGTATACCTGTCAGCACCACAACAGTGGCCAGTTCTGAAATCAACAGCCTTTCAACA
ACTCCTGCTGTCACCAGCACACCTGTGACCACTTATTCTCAAGCCAGTTCATCTCCTACAACTG
CTGACGGTACCAGCATGCAAACCTCAACTTATAGTGAAGGAAGCACTCCACTAACAAGTTTGCC
TGTCAGCACCATGCTGGTGGTCAGTTCTGAGGCTAACACCCTTTCAACAACCCCTATTGACTCC
AAAACTCAGGTGACCGCTTCTACTGAAGCCAGTTCATCTACAACCGCTGAAGGTAGCAGCATGA
CAATCTCAACTCCTAGTGAAGGAAGTCCTCTATTAACAAGTATACCTGTCAGCACCACGCCGGT
GGCCAGTCCTGAGGCTAGCACCCTTTCAACAACTCCTGTTGACTCCAACAGTCCTGTGATCACT
TCTACTGAAGTCAGTTCATCTCCTACACCTGCTGAAGGTACCAGCATGCCAACCTCAACTTATA
CTGAAGGAAGAACTCCTTTAACAAGTATAACTGTCAGAACAACACCGGTGGCCAGCTCTGCAAT
CAGCACCCTTTCAACAACTCCCGTTGACAACAGCACACCTGTGACCACTTCTACTGAAGCCCGT
TCATCTCCTACAACTTCTGAAGGTACCAGCATGCCAAACTCAACTCCTAGTGAAGGAACCACTC
CATTAACAAGTATACCTGTCAGCACCACGCCGGTACTCAGTTCTGAGGCTAGCACCCTTTCAGC
AACTCCTATTGACACCAGCACCCCTGTGACCACTTCTACTGAAGCCACTTCGTCTCCTACAACT
GCTGAAGGTACCAGCATACCAACCTCGACTCTTAGTGAAGGAATGACTCCATTAACAAGCACAC
CTGTCAGCCACACGCTGGTGGCCAATTCTGAGGCTAGCACCCTTTCAACAACTCCTGTTGACTC
TAACAGTCCTGTGGTCACTTCTACAGCAGTCAGTTCATCTCCTACACCTGCTGAAGGTACCAGC
ATAGCAACCTCAACGCCTAGTGAAGGAAGCACTGCATTAACAAGTATACCTGTCAGCACCACAA
CAGTGGCCAGTTCTGAAACCAACACCCTTTCAACAACTCCCGCTGTCACCAGCACACCTGTGAC
CACTTATGCTCAAGTCAGTTCATCTCCTACAACTGCTGACGGTAGCAGCATGCCAACCTCAACT
CCTAGGGAAGGAAGGCCTCCATTAACAAGTATACCTGTCAGCACCACAACAGTGGCCAGTTCTG
AAATCAACACCCTTTCAACAACTCTTGCTGACACCAGGACACCTGTGACCACTTATTCTCAAGC
CAGTTCATCTCCTACAACTGCTGATGGTACCAGCATGCCAACCCCAGCTTATAGTGAAGGAAGC
ACTCCACTAACAAGTATGCCTCTCAGCACCACGCTGGTGGTCAGTTCTGAGGCTAGCACTCTTT
CCACAACTCCTGTTGACACCAGCACTCCTGCCACCACTTCTACTGAAGGCAGTTCATCTCCTAC
AACTGCAGGAGGTACCAGCATACAAACCTCAACTCCTAGTGAACGGACCACTCCATTAGCAGGT
ATGCCTGTCAGCACTACGCTTGTGGTCAGTTCTGAGGGTAACACCCTTTCAACAACTCCTGTTG
ACTCCAAAACTCAGGTGACCAATTCTACTGAAGCCAGTTCATCTGCAACCGCTGAAGGTAGCAG
CATGACAATCTCAGCTCCTAGTGAAGGAAGTCCTCTACTAACAAGTATACCTCTCAGCACCACG
CCGGTGGCCAGTCCTGAGGCTAGCACCCTTTCAACAACTCCTGTTGACTCCAACAGTCCTGTGA
TCACTTCTACTGAAGTCAGTTCATCTCCTATACCTACTGAAGGTACCAGCATGCAAACCTCAAC
TTATAGTGACAGAAGAACTCCTTTAACAAGTATGCCTGTCAGCACCACAGTGGTGGCCAGTTCT
GCAATCAGCACCCTTTCAACAACTCCTGTTGACACCAGCACACCTGTGACCAATTCTACTGAAG
CCCGTTCATCTCCTACAACTTCTGAAGGTACCAGCATGCCAACCTCAACTCCTAGTGAAGGAAG
CACTCCATTCACAAGTATGCCTGTCAGCACCATGCCGGTAGTTACTTCTGAGGCTAGCACCCTT
TCAGCAACTCCTGTTGACACCAGCACACCTGTGACCACTTCTACTGAAGCCACTTCATCTCCTA
CAACTGCTGAAGGTACCAGCATACCAACTTCAACTCTTAGTGAAGGAACGACTCCATTAACAAG
TATACCTGTCAGCCACACGCTGGTGGCCAATTCTGAGGTTAGCACCCTTTCAACAACTCCTGTT
GACTCCAACACTCCTTTCACTACTTCTACTGAAGCCAGTTCACCTCCTCCCACTGCTGAAGGTA
CCAGCATGCCAACCTCAACTTCTAGTGAAGGAAACACTCCATTAACACGTATGCCTGTCAGCAC
CACAATGGTGGCCAGTTTTGAAACAAGCACACTTTCTACAACTCCTGCTGACACCAGCACACCT
GTGACTACTTATTCTCAAGCCGGTTCATCTCCTACAACTGCTGACGATACTAGCATGCCAACCT
CAACTTATAGTGAAGGAAGCACTCCACTAACAAGTGTGCCTGTCAGCACCATGCCGGTGGTCAG
TTCTGAGGCTAGCACCCATTCCACAACTCCTGTTGACACCAGCACACCTGTCACCACTTCTACT
GAAGCCAGTTCATCTCCTACAACTGCTGAAGGTACCAGCATACCAACCTCACCTCCTAGTGAAG
GAACCACTCCGTTAGCAAGTATGCCTGTCAGCACCACGCCGGTGGTCAGTTCTGAGGCTGGCAC
CCTTTCCACAACTCCTGTTGACACCAGCACACCTATGACCACTTCTACTGAAGCCAGTTCATCT
CCTACAACTGCTGAAGATATCGTCGTGCCAATCTCAACTGCTAGTGAAGGAAGTACTCTATTAA
CAAGTATACCTGTCAGCACCACGCCAGTGGCCAGTCCTGAGGCTAGCACCCTTTCAACAACTCC
TGTTGACTCCAACAGTCCTGTGGTCACTTCTACTGAAATCAGTTCATCTGCTACATCCGCTGAA
GGTACCAGCATGCCTACCTCAACTTATAGTGAAGGAAGCACTCCATTAAGAAGTATGCCTGTCA
GCACCAAGCCGTTGGCCAGTTCTGAGGCTAGCACTCTTTCAACAACTCCTGTTGACACCAGCAT
ACCTGTCACCACTTCTACTGAAACCAGTTCATCTCCTACAACTGCAAAAGATACCAGCATGCCA
ATCTCAACTCCTAGTGAAGTAAGTACTTCATTAACAAGTATACTTGTCAGCACCATGCCAGTGG
CCAGTTCTGAGGCTAGCACCCTTTCAACAACTCCTGTTGACACCAGGACACTTGTGACCACTTC
CACTGGAACCAGTTCATCTCCTACAACTGCTGAAGGTAGCAGCATGCCAACCTCAACTCCTGGT
GAAAGAAGCACTCCATTAACAAATATACTTGTCAGCACCACGCTGTTGGCCAATTCTGAGGCTA
GCACCCTTTCAACAACTCCTGTTGACACCAGCACACCTGTCACCACTTCTGCTGAAGCCAGTTC
TTCTCCTACAACTGCTGAAGGTACCAGCATGCGAATCTCAACTCCTAGTGATGGAAGTACTCCA
TTAACAAGTATACTTGTCAGCACCCTGCCAGTGGCCAGTTCTGAGGCTAGCACCGTTTCAACAA
CTGCTGTTGACACCAGCATACCTGTCACCACTTCTACTGAAGCCAGTTCCTCTCCTACAACTGC
TGAAGTTACCAGCATGCCAACCTCAACTCCTAGTGAAACAAGTACTCCATTAACTAGTATGCCT
GTCAACCACACGCCAGTGGCCAGTTCTGAGGCTGGCACCCTTTCAACAACTCCTGTTGACACCA
GCACACCTGTGACCACTTCTACTAAAGCCAGTTCATCTCCTACAACTGCTGAAGGTATCGTCGT
GCCAATCTCAACTGCTAGTGAAGGAAGTACTCTATTAACAAGTATACCTGTCAGCACCACGCCG
GTGGCCAGTTCTGAGGCTAGCACCCTTTCAACAACTCCTGTTGATACCAGCATACCTGTCACCA
CTTCTACTGAAGGCAGTTCTTCTCCTACAACTGCTGAAGGTACCAGCATGCCAATCTCAACTCC
TAGTGAAGTAAGTACTCCATTAACAAGTATACTTGTCAGCACCGTGCCAGTGGCCGGTTCTGAG
GCTAGCACCCTTTCAACAACTCCTGTTGACACCAGGACACCTGTCACCACTTCTGCTGAAGCTA
GTTCTTCTCCTACAACTGCTGAAGGTACCAGCATGCCAATCTCAACTCCTGGCGAAAGAAGAAC
TCCATTAACAAGTATGTCTGTCAGCACCATGCCGGTGGCCAGTTCTGAGGCTAGCACCCTTTCA
AGAACTCCTGCTGACACCAGCACACCTGTGACCACTTCTACTGAAGCCAGTTCCTCTCCTACAA
CTGCTGAAGGTACCGGCATACCAATCTCAACTCCTAGTGAAGGAAGTACTCCATTAACAAGTAT
ACCTGTCAGCACCACGCCAGTGGCCATTCCTGAGGCTAGCACCCTTTCAACAACTCCTGTTGAC
TCCAACAGTCCTGTGGTCACTTCTACTGAAGTCAGTTCATCTCCTACACCTGCTGAAGGTACCA
GCATGCCAATCTCAACTTATAGTGAAGGAAGCACTCCATTAACAGGTGTGCCTGTCAGCACCAC
ACCGGTGACCAGTTCTGCAATCAGCACCCTTTCAACAACTCCTGTTGACACCAGCACACCTGTG
ACCACTTCTACTGAAGCCCATTCATCTCCTACAACTTCTGAAGGTACCAGCATGCCAACCTCAA
CTCCTAGTGAAGGAAGTACTCCATTAACATATATGCCTGTCAGCACCATGCTGGTAGTCAGTTC
TGAGGATAGCACCCTTTCAGCAACTCCTGTTGACACCAGCACACCTGTGACCACTTCTACTGAA
GCCACTTCATCTACAACTGCTGAAGGTACCAGCATTCCAACCTCAACTCCTAGTGAAGGAATGA
CTCCATTAACTAGTGTACCTGTCAGCAACACGCCGGTGGCCAGTTCTGAGGCTAGCATCCTTTC
AACAACTCCTGTTGACTCCAACACTCCTTTGACCACTTCTACTGAAGCCAGTTCATCTCCTCCC
ACTGCTGAAGGTACCAGCATGCCAACCTCAACTCCTAGTGAAGGAAGCACTCCATTAACAAGTA
TGCCTGTCAGCACCACAACGGTGGCCAGTTCTGAAACGAGCACCCTTTCAACAACTCCTGCTGA
CACCAGCACACCTGTGACCACTTATTCTCAAGCCAGTTCATCTCCTCCAATTGCTGACGGTACT
AGCATGCCAACCTCAACTTATAGTGAAGGAAGCACTCCACTAACAAATATGTCTTTCAGCACCA
CGCCAGTGGTCAGTTCTGAGGCTAGCACCCTTTCCACAACTCCTGTTGACACCAGCACACCTGT
CACCACTTCTACTGAAGCCAGTTTATCTCCTACAACTGCTGAAGGTACCAGCATACCAACCTCA
AGTCCTAGTGAAGGAACCACTCCATTAGCAAGTATGCCTGTCAGCACCACGCCGGTGGTCAGTT
CTGAGGTTAACACCCTTTCAACAACTCCTGTGGACTCCAACACTCTGGTGACCACTTCTACTGA
AGCCAGTTCATCTCCTACAATCGCTGAAGGTACCAGCTTGCCAACCTCAACTACTAGTGAAGGA
AGCACTCCATTATCAATTATGCCTCTCAGTACCACGCCGGTGGCCAGTTCTGAGGCTAGCACCC
TTTCAACAACTCCTGTTGACACCAGCACACCTGTGACCACTTCTTCTCCAACCAATTCATCTCC
TACAACTGCTGAAGTTACCAGCATGCCAACATCAACTGCTGGTGAAGGAAGCACTCCATTAACA
AATATGCCTGTCAGCACCACACCGGTGGCCAGTTCTGAGGCTAGCACCCTTTCAACAACTCCTG
TTGACTCCAACACTTTTGTTACCAGTTCTAGTCAAGCCAGTTCATCTCCAGCAACTCTTCAGGT
CACCACTATGCGTATGTCTACTCCAAGTGAAGGAAGCTCTTCATTAACAACTATGCTCCTCAGC
AGCACATATGTGACCAGTTCTGAGGCTAGCACACCTTCCACTCCTTCTGTTGACAGAAGCACAC
CTGTGACCACTTCTACTCAGAGCAATTCTACTCCTACACCTCCTGAAGTTATCACCCTGCCAAT
GTCAACTCCTAGTGAAGTAAGCACTCCATTAACCATTATGCCTGTCAGCACCACATCGGTGACC
ATTTCTGAGGCTGGCACAGCTTCAACACTTCCTGTTGACACCAGCACACCTGTGATCACTTCTA
CCCAAGTCAGTTCATCTCCTGTGACTCCTGAAGGTACCACCATGCCAATCTGGACGCCTAGTGA
AGGAAGCACTCCATTAACAACTATGCCTGTCAGCACCACACGTGTGACCAGCTCTGAGGGTAGC
ACCCTTTCAACACCTTCTGTTGTCACCAGCACACCTGTGACCACTTCTACTGAAGCCATTTCAT
CTTCTGCAACTCTTGACAGCACCACCATGTCTGTGTCAATGCCCATGGAAATAAGCACCCTTGG
GACCACTATTCTTGTCAGTACCACACCTGTTACGAGGTTTCCTGAGAGTAGCACCCCTTCCATA
CCATCTGTTTACACCAGCATGTCTATGACCACTGCCTCTGAAGGCAGTTCATCTCCTACAACTC
TTGAAGGCACCACCACCATGCCTATGTCAACTACGAGTGAAAGAAGCACTTTATTGACAACTGT
CCTCATCAGCCCTATATCTGTGATGAGTCCTTCTGAGGCCAGCACACTTTCAACACCTCCTGGT
GATACCAGCACACCTTTGCTCACCTCTACCAAAGCCGGTTCATTCTCCATACCTGCTGAAGTCA
CTACCATACGTATTTCAATTACCAGTGAAAGAAGCACTCCATTAACAACTCTCCTTGTCAGCAC
CACACTTCCAACTAGCTTTCCTGGGGCCAGCATAGCTTCGACACCTCCTCTTGACACAAGCACA
ACTTTTACCCCTTCTACTGACACTGCCTCAACTCCCACAATTCCTGTAGCCACCACCATATCTG
TATCAGTGATCACAGAAGGAAGCACACCTGGGACAACCATTTTTATTCCCAGCACTCCTGTCAC
CAGTTCTACTGCTGATGTCTTTCCTGCAACAACTGGTGCTGTATCTACCCCTGTGATAACTTCC
ACTGAACTAAACACACCATCAACCTCCAGTAGTAGTACCACCACATCTTTTTCAACTACTAAGG
AATTTACAACACCCGCAATGACTACTGCAGCTCCCCTCACATATGTGACCATGTCTACTGCCCC
CAGCACACCCAGAACAACCAGCAGAGGCTGCACTACTTCTGCATCAACGCTTTCTGCAACCAGT
ACACCTCACACCTCTACTTCTGTCACCACCCGTCCTGTGACCCCTTCATCAGAATCCAGCAGGC
CGTCAACAATTACTTCTCACACCATCCCACCTACATTTCCTCCTGCTCACTCCAGTACACCTCC
AACAACCTCTGCCTCCTCCACGACTGTGAACCCTGAGGCTGTCACCACCATGACCACCAGGACA
AAACCCAGCACACGGACCACTTCCTTCCCCACGGTGACCACCACCGCTGTCCCCACGAATACTA
CAATTAAGAGCAACCCCACCTCAACTCCTACTGTGCCAAGAACCACAACATGCTTTGGAGATGG
GTGCCAGAATACGGCCTCTCGCTGCAAGAATGGAGGCACCTGGGATGGGCTCAAGTGCCAGTGT
CCCAACCTCTATTATGGGGAGTTGTGTGAGGAGGTGGTCAGCAGCATTGACATAGGGCCACCGG
AGACTATCTCTGCCCAAATGGAACTGACTGTGACAGTGACCAGTGTGAAGTTCACCGAAGAGCT
AAAAAACCACTCTTCCCAGGAATTCCAGGAGTTCAAACAGACATTCACGGAACAGATGAATATT
GTGTATTCCGGGATCCCTGAGTATGTCGGGGTGAACATCACAAAGCTACGACATGATGTGTTTC
AACACCACTGGCACCCAAGTGCAAAACATTACGGTGACCCAGTACGACCCTGAagaggactgcc
ggaagatggccaaggaatatggagactacttcgtagtggagtaccgggaccagaagccatactg
catcagcccctgtgagcctggcttcagtgtctccaagaactgtaacctcggcaagtgccagatg
tctctaagtggacctcagtgcctctgcgtgaccacggaaactcactggtacagtggggagacct
gtaaccagggcacccagaagagtctggtgtacggcctcgtgggggcaggggtcgtgctgatgct
gatcatcctggtagctctcctgatgctcgttttccgctccaagagagaggtgaaacggcaaaag
tacagattgtctcagttatacaagtggcaagaagaggacagtggaccagctcctgggaccttcc
aaaacattggctttgacatctgccaagatgatgattccatccacctggagtccatctatagtaa
tttccagccctccttgagacacatagaccctgaaacaaagatccgaattcagaggcctcaggta
atgacgacatcattttaaggcatggagctgagaagtctgggagtgaggagatcccagtccggct
aagcttggtggagcattttcccattgagagccttccatgggaactcaatgttcccattgtaagt
acaggaaacaagccctgtacttaccaaggagaaagaggagagacagcagtgctgggagattctc
aaatagaaacccgtggacgctccaatgggcttgtcatgatatcaggctaggctttcctgctcat
ttttcaaagacgctccagatttgagggtactctgactgcaacatctttcaccccattgatcgcc
aggattgatttggttgatctggctgagcaggcgggtgtccccgtcctccctcactgccccatat
gtgtccctcctaaagctgcatgctcagttgaagaggacgagaggacgaccttctctgatagagg
aggaccacgcttcagtcaaaggcatacaagtatctatctggacttccctgctagcacttccaaa
caagctcagagatgttcctcccctcatctgcccgggttcagtaccatggacagcgccctcgacc
cgctgtttacaaccatgaccccttggacactggactgcatgcactttacatatcacaaaatgct
ctcataagaattattgcataccatcttcatgaaaaacacctgtatttaaatatagagcatttac
cttttggta
SEQ ID NO: 6 = Ensembl polypeptide sequence of human MUC17
(4262 amino acids)
MPRPGTMALCLLTLVLSLLPPQAAAEQDLSVNRAVWDGGGCISQGDVLNRQCQQLSQHVRTGSA
ANTATGTTSTNVVEPRMYLSCSTNPEMTSIESSVTSDTPGVSSTRMTPTESRTTSESTSDSTTL
FPSSTEDTSSPTTPEGTDVPMSTPSEESISSTMAFVSTAPLPSFEAYTSLTYKVDMSTPLTTST
QASSSPTTPESTTIPKSTNSEGSTPLTSMPASTMKVASSEAITLLTTPVEISTPVTISAQASSS
PTTAEGPSLSNSAPSGGSTPLTRMPLSVMLVVSSEASTLSTTPAATNIPVITSTEASSSPTTAE
GTSIPTSTYTEGSTPLTSTPASTMPVATSEMSTLSITPVDTSTLVTTSTEPSSLPTTAEATSML
TSTLSEGSTPLTNMPVSTILVASSEASTTSTIPVDSKTFVTTASEASSSPTTAEDTSIATSTPS
EGSTPLTSMPVSTTPVASSEASNLSTTPVDSKTQVTTSTEASSSPPTAEVNSMPTSTPSEGSTP
LTSMSVSTMPVASSEASTLSTTPVDTSTPVTTSSEASSSSTTPEGTSIPTSTPSEGSTPLTNMP
VSTRLVVSSEASTTSTTPADSNTFVTTSSEASSSSTTAEGTSMPTSTYSERGTTITSMSVSTTL
VASSEASTLSTTPVDSNTPVTTSTEATSSSTTAEGTSMPTSTYTEGSTPLTSMPVNTTLVASSE
ASTLSTTPVDTSTPVTTSTEASSSPTTADGASMPTSTPSEGSTPLTSMPVSKTLLTSSEASTLS
TTPLDTSTHITTSTEASCSPTTTEGTSMPISTPSEGSPLLTSIPVSITPVTSPEASTLSTTPVD
SNSPVTTSTEVSSSPTPAEGTSMPTSTYSEGRTPLTSMPVSTTLVATSAISTLSTTPVDTSTPV
TNSTEARSSPTTSEGTSMPTSTPGEGSTPLTSMPDSTTPVVSSEARTLSATPVDTSTPVTTSTE
ATSSPTTAEGTSIPTSTPSEGTTPLTSTPVSHTLVANSEASTLSTTPVDSNTPLTTSTEASSPP
PTAEGTSMPTSTPSEGSTPLTRMPVSTTMVASSETSTLSTTPADTSTPVTTYSQASSSSTTADG
TSMPTSTYSEGSTPLTSVPVSTRLVVSSEASTLSTTPVDTSIPVTTSTEASSSPTTAEGTSIPT
SPPSEGTTPLASMPVSTTLVVSSEANTLSTTPVDSKTQVATSTEASSPPPTAEVTSMPTSTPGE
RSTPLTSMPVRHTPVASSEASTLSTSPVDTSTPVTTSAETSSSPTTAEGTSLPTSTTSEGSTLL
TSIPVSTTLVTSPEASTLLTTPVDTKGPVVTSNEVSSSPTPAEGTSMPTSTYSEGRTPLTSIPV
NTTLVASSAISILSTTPVDNSTPVTTSTEACSSPTTSEGTSMPNSNPSEGTTPLTSIPVSTTPV
VSSEASTLSATPVDTSTPGTTSAEATSSPTTAEGISIPTSTPSEGKTPLKSIPVSNTPVANSEA
STLSTTPVDSNSPVVTSTAVSSSPTPAEGTSIAISTPSEGSTALTSIPVSTTTVASSEINSLST
TPAVTSTPVTTYSQASSSPTTADGTSMQTSTYSEGSTPLTSLPVSTMLVVSSEANTLSTTPIDS
KTQVTASTEASSSTTAEGSSMTISTPSEGSPLLTSIPVSTTPVASPEASTLSTTPVDSNSPVIT
STEVSSSPTPAEGTSMPTSTYTEGRTPLTSITVRTTPVASSAISTLSTTPVDNSTPVTTSTEAR
SSPTTSEGTSMPNSTPSEGTTPLTSIPVSTTPVLSSEASTLSATPIDTSTPVTTSTEATSSPTT
AEGTSIPTSTLSEGMTPLTSTPVSHTLVANSEASTLSTTPVDSNSPVVTSTAVSSSPTPAEGTS
IATSTPSEGSTALTSIPVSTTTVASSETNTLSTTPAVTSTPVTTYAQVSSSPTTADGSSMPTST
PREGRPPLTSIPVSTTTVASSEINTLSTTLADTRTPVTTYSQASSSPTTADGTSMPTPAYSEGS
TPLTSMPLSTTLVVSSEASTLSTTPVDTSTPATTSTEGSSSPTTAGGTSIQTSTPSERTTPLAG
MPVSTTLVVSSEGNTLSTTPVDSKTQVTNSTEASSSATAEGSSMTISAPSEGSPLLTSIPLSTT
PVASPEASTLSTTPVDSNSPVITSTEVSSSPIPTEGTSMQTSTYSDRRTPLTSMPVSTTVVASS
AISTLSTTPVDTSTPVTNSTEARSSPTTSEGTSMPTSTPSEGSTPFTSMPVSTMPVVTSEASTL
SATPVDTSTPVTTSTEATSSPTTAEGTSIPTSTLSEGTTPLTSIPVSHTLVANSEVSTLSTTPV
DSNTPFTTSTEASSPPPTAEGTSMPTSTSSEGNTPLTRMPVSTTMVASFETSTLSTTPADTSTP
VTTYSQAGSSPTTADDTSMPTSTYSEGSTPLTSVPVSTMPVVSSEASTHSTTPVDTSTPVTTST
EASSSPTTAEGTSIPTSPPSEGTTPLASMPVSTTPVVSSEAGTLSTTPVDTSTPMTTSTEASSS
PTTAEDIVVPISTASEGSTLLTSIPVSTTPVASPEASTLSTTPVDSNSPVVTSTEISSSATSAE
GTSMPTSTYSEGSTPLRSMPVSTKPLASSEASTLSTTPVDTSIPVTTSTETSSSPTTAKDTSMP
ISTPSEVSTSLTSILVSTMPVASSEASTLSTTPVDTRTLVTTSTGTSSSPTTAEGSSMPTSTPG
ERSTPLTNILVSTTLLANSEASTLSTTPVDTSTPVTTSAEASSSPTTAEGTSMRISTPSDGSTP
LTSILVSTLPVASSEASTVSTTAVDTSIPVTTSTEASSSPTTAEVTSMPTSTPSETSTPLTSMP
VNHTPVASSEAGTLSTTPVDTSTPVTTSTKASSSPTTAEGIVVPISTASEGSTLLTSIPVSTTP
VASSEASTLSTTPVDTSIPVTTSTEGSSSPTTAEGTSMPISTPSEVSTPLTSILVSTVPVAGSE
ASTLSTTPVDTRTPVTTSAEASSSPTTAEGTSMPISTPGERRTPLTSMSVSTMPVASSEASTLS
RTPADTSTPVTTSTEASSSPTTAEGTGIPISTPSEGSTPLTSIPVSTTPVAIPEASTLSTTPVD
SNSPVVTSTEVSSSPTPAEGTSMPISTYSEGSTPLTGVPVSTTPVTSSAISTLSTTPVDTSTPV
TTSTEAHSSPTTSEGTSMPTSTPSEGSTPLTYMPVSTMLVVSSEDSTLSATPVDTSTPVTTSTE
ATSSTTAEGTSIPTSTPSEGMTPLTSVPVSNTPVASSEASILSTTPVDSNTPLTTSTEASSSPP
TAEGTSMPTSTPSEGSTPLTSMPVSTTTVASSETSTLSTTPADTSTPVTTYSQASSSPPIADGT
SMPTSTYSEGSTPLTNMSFSTTPVVSSEASTLSTTPVDTSTPVTTSTEASLSPTTAEGTSIPTS
SPSEGTTPLASMPVSTTPVVSSEVNTLSTTPVDSNTLVTTSTEASSSPTIAEGTSLPTSTTSEG
STPLSIMPLSTTPVASSEASTLSTTPVDTSTPVTTSSPTNSSPTTAEVTSMPTSTAGEGSTPLT
NMPVSTTPVASSEASTLSTTPVDSNTFVTSSSQASSSPATLQVTTMRMSTPSEGSSSLTTMLLS
STYVTSSEASTPSTPSVDRSTPVTTSTQSNSTPTPPEVITLPMSTPSEVSTPLTIMPVSTTSVT
ISEAGTASTLPVDTSTPVITSTQVSSSPVTPEGTTMPIWTPSEGSTPLTTMPVSTTRVTSSEGS
TLSTPSVVTSTPVTTSTEAISSSATLDSTTMSVSMPMEISTLGTTILVSTTPVTRFPESSTPSI
PSVYTSMSMTTASEGSSSPTTLEGTTTMPMSTTSERSTLLTTVLISPISVMSPSEASTLSTPPG
DTSTPLLTSTKAGSFSIPAEVTTIRISITSERSTPLTTLLVSTTLPTSFPGASIASTPPLDTST
TFTPSTDTASTPTIPVATTISVSVITEGSTPGTTIFIPSTPVTSSTADVFPATTGAVSTPVITS
TELNTPSTSSSSTTTSFSTTKEFTTPAMTTAAPLTYVTMSTAPSTPRTTSRGCTTSASTLSATS
TPHTSTSVTTRPVTPSSESSRPSTITSHTIPPTFPPAHSSTPPTTSASSTTVNPEAVTTMTTRT
KPSTRTTSFPTVTTTAVPTNTTIKSNPTSTPTVPRTTTCFGDGCQNTASRCKNGGTWDGLKCQC
PNLYYGELCEEVVSSIDIGPPETISAQMELTVTVTSVKFTEELKNHSSQEFQEFKQTFTEQMNI
VYSGIPEYVGVNITKLRHDVFQHHWHPSAKHYGDPVRP
SEQ ID NO: 7 = RefSeq nucleotide sequence encoding human VSIG1 (mRNA)
aaagtctatacgcaataagtaagcccaaagaggcatgtttgcttggcgatgcccagcagataag
ccaggcaaacctcggtgtgatcgaagaagccaatttgagactcagcctagtccaggcaagctac
tggcacctgctgctctcaactaacctccacacaatggtgttcgcattttggaaggtctttctga
tcctaagctgccttgcaggtcaggttagtgtggtgcaagtgaccatcccagacggtttcgtgaa
cgtgactgttggatctaatgtcactctcatctgcatctacaccaccactgtggcctcccgagaa
cagctttccatccagtggtctttcttccataagaaggagatggagccaatttctcacagctcgt
gcctcagtactgagggtatggaggaaaaggcagtcagtcagtgtctaaaaatgacgcacgcaag
agacgctcggggaagatgtagctggacctctgagatttacttttctcaaggtggacaagctgta
gccatcgggcaatttaaagatcgaattacagggtccaacgatccaggtaatgcatctatcacta
tctcgcatatgcagccagcagacagtggaatttacatctgcgatgttaacaaccccccagactt
tctcggccaaaaccaaggcatcctcaacgtcagtgtgttagtgaaaccttctaagcccctttgt
agcgttcaaggaagaccagaaactggccacactatttccctttcctgtctctctgcgcttggaa
caccttcccctgtgtactactggcataaacttgagggaagagacatcgtgccagtgaaagaaaa
cttcaacccaaccaccgggattttggtcattggaaatctgacaaattttgaacaaggttattac
cagtgtactgccatcaacagacttggcaatagttcctgcgaaatcgatctcacttcttcacatc
cagaagttggaatcattgttggggccttgattggtagcctggtaggtgccgccatcatcatctc
tgttgtgtgcttcgcaaggaataaggcaaaagcaaaggcaaaagaaagaaattctaagaccatc
gcggaacttgagccaatgacaaagataaacccaaggggagaaagcgaagcaatgccaagagaag
acgctacccaactagaagtaactctaccatcttccattcatgagactggccctgataccatcca
agaaccagactatgagccaaagcctactcaggagcctgccccagagcctgccccaggatcagag
cctatggcagtgcctgaccttgacatcgagctggagctggagccagaaacgcagtcggaattgg
agccagagccagagccagagccagagtcagagcctggggttgtagttgagcccttaagtgaaga
tgaaaagggagtggttaaggcataggctggtggcctaagtacagcattaatcattaaggaaccc
attactgccatttggaattcaaataacctaaccaacctccacctcctccttccattttgaccaa
ccttcttctaacaaggtgctcattcctactatgaatccagaataaacacgccaagataacagct
aaatcagcaagggttcctgtattaccaatatagaatactaacaattttactaacacgtaagcat
aacaaatgacagggcaagtgatttctaacttagttgagttttgcaacagtacctgtgttgttat
ttcagaaaatattatttctctctttttaactactctttttttttattttagacagagtcttgct
ccgtcgcgcaggctgtgatcgtagtggtgcgatctcggctcactgcaacctccgctccctgggt
tcaagcgattctcctgcctgagcctcctgagtagctgggactacaggcacgtgccaccacgccc
ggctaattttttgtatttttagtagagatggggtttcacgttgttagccaggatggtctccatc
tcctgacctcatgatccgcccaccttggcctcccaaaatgctgggattacaggcatgagccact
gcgcccggcctctttttagctactcttatgttccacatgcacatatgacaaggtggcattaatt
agattcaatattatttctaggaatagttcctcattcatttttatattgaccactaagaaaataa
ttcatcagcattatctcatagattggaaaattttctccaaatacaatagaggagaatatgtaaa
gggtatacattaattggtacgtagcatttaaaatcaggtcttataattaatgcttcattcctca
tattagatttcccaagaaatcaccctggtatccaatatctgagcatggcaaatttaaaaaataa
cacaatttcttgcctgtaaccctagcactttgggaggccgaggcaggtggatcacctgaggtca
ggagttcgagaccagcctggccaacatggcgaaaccccttctctactaaaaatacaaaaattag
ctgggcgtggtagtgcatgcctgtaatcccagctacttgggaggctgaggcaggagaatcgctt
gaacccaggaggtggaggttgcagtgagccgagattgtgccactgcactccaacctgggtgaca
gagtgagattccatctgaaaaacaaaaacaaaaacagaaaacaaacaaacaaaaaacaaaaaat
ccccacaactttgtcaaataatgtacaggcaaacactttcaaatataatttccttcagtgaata
caaaatgttgatatcataggtgatgtacaatttagttttgaatgagttattatgttatcactgt
gtctgatgttatctactttgaaaggcagtccagaaaagtgttctaagtgaactcttaagatcta
ttttagataatttcaactaattaaataacctgttttactgcctgtacattccacattaataaag
cgataccaatcttatatgaatgctaatattactaaaatgcactgatatcacttcttcttcccct
gttgaaaagctttctcatgatcatatttcacccacatctcaccttgaagaaacttacaggtaga
cttaccttttcacttgtggaattaatcatatttaaatcttactttaaggctcaataaataatac
tcataatgtctcattttagtgactcctaaggctagtccttttataaacaactttttctgacata
gcatttatgtataataaaccagacatttaaagtgta
SEQ ID NO: 8 = RefSeq polypeptide sequence of human VSIG1
(423 amino acids)
MVFAFWKVFLILSCLAGQVSVVQVTIPDGFVNVTVGSNVTLICIYTTTVASREQLSIQWSFFHK
KEMEPISHSSCLSTEGMEEKAVSQCLKMTHARDARGRCSWTSEIYFSQGGQAVAIGQFKDRITG
SNDPGNASITISHMQPADSGIYICDVNNPPDFLGQNQGILNVSVLVKPSKPLCSVQGRPETGHT
ISLSCLSALGTPSPVYYWHKLEGRDIVPVKENFNPTTGILVIGNLTNFEQGYYQCTAINRLGNS
SCEIDLTSSHPEVGIIVGALIGSLVGAAIIISVVCFARNKAKAKAKERNSKTIAELEPMTKINP
RGESEAMPREDATQLEVTLPSSIHETGPDTIQEPDYEPKPTQEPAPEPAPGSEPMAVPDLDIEL
ELEPETQSELEPEPEPEPESEPGVVVEPLSEDEKGVVKA
SEQ ID NO: 9 = Ensembl nucleotide sequence encoding human VSIG1 (mRNA)
aaagtctatacgcaataagtaagcccaaagaggcatgtttgcttggcgatgcccagcagataag
ccaggcaaacctcggtgtgatcgaagaagccaatttgagactcagcctagtccaggcaagctac
tggcacctgctgctctcaactaacctccacacaATGGTGTTCGCATTTTGGAAGGTCTTTCTGA
TCCTAAGCTGCCTTGCAGGTCAGGTTAGTGTGGTGCAAGTGACCATCCCAGACGGTTTCGTGAA
CGTGACTGTTGGATCTAATGTCACTCTCATCTGCATCTACACCACCACTGTGGCCTCCCGAGAA
CAGCTTTCCATCCAGTGGTCTTTCTTCCATAAGAAGGAGATGGAGCCAATTTCTCACAGCTCGT
GCCTCAGTACTGAGGGTATGGAGGAAAAGGCAGTCAGTCAGTGTCTAAAAATGACGCACGCAAG
AGACGCTCGGGGAAGATGTAGCTGGACCTCTGAGATTTACTTTTCTCAAGGTGGACAAGCTGTA
GCCATCGGGCAATTTAAAGATCGAATTACAGGGTCCAACGATCCAGGTAATGCATCTATCACTA
TCTCGCATATGCAGCCAGCAGACAGTGGAATTTACATCTGCGATGTTAACAACCCCCCAGACTT
TCTCGGCCAAAACCAAGGCATCCTCAACGTCAGTGTGTTAGTGAAACCTTCTAAGCCCCTTTGT
AGCGTTCAAGGAAGACCAGAAACTGGCCACACTATTTCCCTTTCCTGTCTCTCTGCGCTTGGAA
CACCTTCCCCTGTGTACTACTGGCATAAACTTGAGGGAAGAGACATCGTGCCAGTGAAAGAAAA
CTTCAACCCAACCACCGGGATTTTGGTCATTGGAAATCTGACAAATTTTGAACAAGGTTATTAC
CAGTGTACTGCCATCAACAGACTTGGCAATAGTTCCTGCGAAATCGATCTCACTTCTTCACATC
CAGAAGTTGGAATCATTGTTGGGGCCTTGATTGGTAGCCTGGTAGGTGCCGCCATCATCATCTC
TGTTGTGTGCTTCGCAAGGAATAAGGCAAAAGCAAAGGCAAAAGAAAGAAATTCTAAGACCATC
GCGGAACTTGAGCCAATGACAAAGATAAACCCAAGGGGAGAAAGCGAAGCAATGCCAAGAGAAG
ACGCTACCCAACTAGAAGTAACTCTACCATCTTCCATTCATGAGACTGGCCCTGATACCATCCA
AGAACCAGACTATGAGCCAAAGCCTACTCAGGAGCCTGCCCCAGAGCCTGCCCCAGGATCAGAG
CCTATGGCAGTGCCTGACCTTGACATCGAGCTGGAGCTGGAGCCAGAAACGCAGTCGGAATTGG
AGCCAGAGCCAGAGCCAGAGCCAGAGTCAGAGCCTGGGGTTGTAGTTGAGCCCTTAAGTGAAGA
TGAAAAGGGAGTGGTTAAGGCATAGgctggtggcctaagtacagcattaatcattaaggaaccc
attactgccatttggaattcaaataacctaaccaacctccacctcctccttccattttgaccaa
ccttcttctaacaaggtgctcattcctactatgaatccagaataaacacgccaagataacagct
aaatcagcaagggttcctgtattaccaatatagaatactaacaattttactaacacgtaagcat
aacaaatgacagggcaagtgatttctaacttagttgagttttgcaacagtacctgtgttgttat
ttcagaaaatattatttctctctttttaactactctttttttttattttagacagagtcttgct
ccgtcgcgcaggctgtgatcgtagtggtgcgatctcggctcactgcaacctccgctccctgggt
tcaagcgattctcctgcctgagcctcctgagtagctgggactacaggcacgtgccaccacgccc
ggctaattttttgtatttttagtagagatggggtttcacgttgttagccaggatggtctccatc
tcctgacctcatgatccgcccaccttggcctcccaaaatgctgggattacaggcatgagccact
gcgcccggcctctttttagctactcttatgttccacatgcacatatgacaaggtggcattaatt
agattcaatattatttctaggaatagttcctcattcatttttatattgaccactaagaaaataa
ttcatcagcattatctcatagattggaaaattttctccaaatacaatagaggagaatatgtaaa
gggtatacattaattggtacgtagcatttaaaatcaggtcttataattaatgcttcattcctca
tattagatttcccaagaaatcaccctggtatccaatatctgagcatggcaaatttaaaaaataa
cacaatttcttgcctgtaaccctagcactttgggaggccgaggcaggtggatcacctgaggtca
ggagttcgagaccagcctggccaacatggcgaaaccccttctctactaaaaatacaaaaattag
ctgggcgtggtagtgcatgcctgtaatcccagctacttgggaggctgaggcaggagaatcgctt
gaacccaggaggtggaggttgcagtgagccgagattgtgccactgcactccaacctgggtgaca
gagtgagattccatctgaaaaacaaaaacaaaaacagaaaacaaacaaacaaaaaacaaaaaat
ccccacaactttgtcaaataatgtacaggcaaacactttcaaatataatttccttcagtgaata
caaaatgttgatatcataggtgatgtacaatttagttttgaatgagttattatgttatcactgt
gtctgatgttatctactttgaaaggcagtccagaaaagtgttctaagtgaactcttaagatcta
ttttagataatttcaactaattaaataacctgttttactgcctgtacattccacattaataaag
cgataccaatcttatatgaatgctaatattactaaaatgcactgatatcacttcttcttcccct
gttgaaaagctttctcatgatcatatttcacccacatctcaccttgaagaaacttacaggtaga
cttaccttttcacttgtggaattaatcatatttaaatcttactttaaggctcaataaataatac
tcataatgtctcattttagtgactcctaaggctagtccttttataaacaactttttctgacata
gcatttatgtataataaaccagacatttaaagtgta
SEQ ID NO: 10 = Ensembl polypeptide sequence of human VSIG1
(423 amino acids)
MVFAFWKVFLILSCLAGQVSVVQVTIPDGFVNVTVGSNVTLICIYTTTVASREQLSIQWSFFHK
KEMEPISHSSCLSTEGMEEKAVSQCLKMTHARDARGRCSWTSEIYFSQGGQAVAIGQFKDRITG
SNDPGNASITISHMQPADSGIYICDVNNPPDFLGQNQGILNVSVLVKPSKPLCSVQGRPETGHT
ISLSCLSALGTPSPVYYWHKLEGRDIVPVKENFNPTTGILVIGNLTNFEQGYYQCTAINRLGNS
SCEIDLTSSHPEVGIIVGALIGSLVGAAIIISVVCFARNKAKAKAKERNSKTIAELEPMTKINP
RGESEAMPREDATQLEVTLPSSIHETGPDTIQEPDYEPKPTQEPAPEPAPGSEPMAVPDLDIEL
ELEPETQSELEPEPEPEPESEPGVVVEPLSEDEKGVVKA
SEQ ID NO: 11 = RefSeq nucleotide sequence encoding human CTSE (mRNA)
atcattcggccctcagactgggctgggcaggtctgagagttagggaaagtccgttcccactgcc
ctcggggagagaagaaaggagggggcaagggagaagctgctggtcggactcacaatgaaaacgc
tccttcttttgctgctggtgctcctggagctgggagaggcccaaggatcccttcacagggtgcc
cctcaggaggcatccgtccctcaagaagaagctgcgggcacggagccagctctctgagttctgg
aaatcccataatttggacatgatccagttcaccgagtcctgctcaatggaccagagtgccaagg
aacccctcatcaactacttggatatggaatacttcggcactatctccattggctccccaccaca
gaacttcactgtcatcttcgacactggctcctccaacctctgggtcccctctgtgtactgcact
agcccagcctgcaagacgcacagcaggttccagccttcccagtccagcacatacagccagccag
gtcaatctttctccattcagtatggaaccgggagcttgtccgggatcattggagccgaccaagt
ctctgtggaaggactaaccgtggttggccagcagtttggagaaagtgtcacagagccaggccag
acctttgtggatgcagagtttgatggaattctgggcctgggatacccctccttggctgtgggag
gagtgactccagtatttgacaacatgatggctcagaacctggtggacttgccgatgttttctgt
ctacatgagcagtaacccagaaggtggtgcggggagcgagctgatttttggaggctacgaccac
tcccatttctctgggagcctgaattgggtcccagtcaccaagcaagcttactggcagattgcac
tggataacatccaggtgggaggcactgttatgttctgctccgagggctgccaggccattgtgga
cacagggacttccctcatcactggcccttccgacaagattaagcagctgcaaaacgccattggg
gcagcccccgtggatggagaatatgctgtggagtgtgccaaccttaacgtcatgccggatgtca
ccttcaccattaacggagtcccctataccctcagcccaactgcctacaccctactggacttcgt
ggatggaatgcagttctgcagcagtggctttcaaggacttgacatccaccctccagctgggccc
ctctggatcctgggggatgtcttcattcgacagttttactcagtctttgaccgtgggaataacc
gtgtgggactggccccagcagtcccctaaggaggggccttgtgtctgtgcctgcctgtctgaca
gaccttgaatatgttaggctggggcattctttacacctacaaaaagttattttccagagaatgt
agctgtttccagggttgcaacttgaattaagaccaaacagaacatgagaatacacacacacaca
cacatatacacacacacacacttcacacatacacaccactcccaccaccgtcatgatggaggaa
ttacgttatacattcatattttgtattgatttttgattatgaaaatcaaaaattttcacatttg
attatgaaaatctccaaacatatgcacaagcagagatcatggtataataaatccctttgcaact
ccactcagccctgacaacccatccacacacggccaggcctgtttatctacactgctgcccactc
ctctctccagctccacatgctgtacctggatcattctgaagcaaattccgagcattacatcatt
ttgtccataaatatttctaacatccttaaatatacaatcggaattcaagcatctcccattgtcc
cacaaatgtttggctgtttttgtagttggattgtttgtattaggattcaagcaaggcccatata
ttgcatttatttgaaatgtctgtaagtctctttccatctacagagtttagcacatttgaacgtt
gctggttgaaatcccgaggtgtcatttgacatggttctctgaacttatctttcctataaaatgg
tagttagatctggaggtctgattttgtggcaaaaatacttcctaggtggtgctgggtacttctt
gttgcatcctgtcaggaggcagataatgctggtgcctctctattggtaatgttaagactgctgg
gtgggtttggagttcttggctttaatcattcattacaaagttcagcattttaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaaa
SEQ ID NO: 12 = RefSeq polypeptide sequence of human CTSE
(396 amino acids)
MKTLLLLLLVLLELGEAQGSLHRVPLRRHPSLKKKLRARSQLSEFWKSHNLDMIQFTESCSMDQ
SAKEPLINYLDMEYFGTISIGSPPQNFTVIFDTGSSNLWVPSVYCTSPACKTHSRFQPSQSSTY
SQPGQSFSIQYGTGSLSGIIGADQVSVEGLTVVGQQFGESVTEPGQTFVDAEFDGILGLGYPSL
AVGGVTPVFDNMMAQNLVDLPMFSVYMSSNPEGGAGSELIFGGYDHSHFSGSLNWVPVTKQAYW
QIALDNIQVGGTVMFCSEGCQAIVDTGTSLITGPSDKIKQLQNAIGAAPVDGEYAVECANLNVM
PDVTFTINGVPYTLSPTAYTLLDFVDGMQFCSSGFQGLDIHPPAGPLWILGDVFIRQFYSVFDR
GNNRVGLAPAVP
SEQ ID NO: 13 = Ensembl nucleotide sequence encoding human CTSE (mRNA)
atcattcggccctcagactgggctgggcaggtctgagagttagggaaagtccgttcccactgcc
ctcggggagagaagaaaggagggggcaagggagaagctgctggtcggactcacaATGAAAACGC
TCCTTCTTTTGCTGCTGGTGCTCCTGGAGCTGGGAGAGGCCCAAGGATCCCTTCACAGGGTGCC
CCTCAGGAGGCATCCGTCCCTCAAGAAGAAGCTGCGGGCACGGAGCCAGCTCTCTGAGTTCTGG
AAATCCCATAATTTGGACATGATCCAGTTCACCGAGTCCTGCTCAATGGACCAGAGTGCCAAGG
AACCCCTCATCAACTACTTGGATATGGAATACTTCGGCACTATCTCCATTGGCTCCCCACCACA
GAACTTCACTGTCATCTTCGACACTGGCTCCTCCAACCTCTGGGTCCCCTCTGTGTACTGCACT
AGCCCAGCCTGCAAGACGCACAGCAGGTTCCAGCCTTCCCAGTCCAGCACATACAGCCAGCCAG
GTCAATCTTTCTCCATTCAGTATGGAACCGGGAGCTTGTCCGGGATCATTGGAGCCGACCAAGT
CTCTGTGGAAGGACTAACCGTGGTTGGCCAGCAGTTTGGAGAAAGTGTCACAGAGCCAGGCCAG
ACCTTTGTGGATGCAGAGTTTGATGGAATTCTGGGCCTGGGATACCCCTCCTTGGCTGTGGGAG
GAGTGACTCCAGTATTTGACAACATGATGGCTCAGAACCTGGTGGACTTGCCGATGTTTTCTGT
CTACATGAGCAGTAACCCAGAAGGTGGTGCGGGGAGCGAGCTGATTTTTGGAGGCTACGACCAC
TCCCATTTCTCTGGGAGCCTGAATTGGGTCCCAGTCACCAAGCAAGCTTACTGGCAGATTGCAC
TGGATAACATCCAGGTGGGAGGCACTGTTATGTTCTGCTCCGAGGGCTGCCAGGCCATTGTGGA
CACAGGGACTTCCCTCATCACTGGCCCTTCCGACAAGATTAAGCAGCTGCAAAACGCCATTGGG
GCAGCCCCCGTGGATGGAGAATATGCTGTGGAGTGTGCCAACCTTAACGTCATGCCGGATGTCA
CCTTCACCATTAACGGAGTCCCCTATACCCTCAGCCCAACTGCCTACACCCTACTGGACTTCGT
GGATGGAATGCAGTTCTGCAGCAGTGGCTTTCAAGGACTTGACATCCACCCTCCAGCTGGGCCC
CTCTGGATCCTGGGGGATGTCTTCATTCGACAGTTTTACTCAGTCTTTGACCGTGGGAATAACC
GTGTGGGACTGGCCCCAGCAGTCCCCTAAggaggggccttgtgtctgtgcctgcctgtctgaca
gaccttgaatatgttaggctggggcattctttacacctacaaaaagttattttccagagaatgt
agctgtttccagggttgcaacttgaattaagaccaaacagaacatgagaatacacacacacaca
cacatatacacacacacacacttcacacatacacaccactcccaccaccgtcatgatggaggaa
ttacgttatacattcatattttgtattgatttttgattatgaaaatcaaaaattttcacatttg
attatgaaaatctccaaacatatgcacaagcagagatcatggtataataaatccctttgcaact
ccactcagccctgacaacccatccacacacggccaggcctgtttatctacactgctgcccactc
ctctctccagctccacatgctgtacctggatcattctgaagcaaattccgagcattacatcatt
ttgtccataaatatttctaacatccttaaatatacaatcggaattcaagcatctcccattgtcc
cacaaatgtttggctgtttttgtagttggattgtttgtattaggattcaagcaaggcccatata
ttgcatttatttgaaatgtctgtaagtctctttccatctacagagtttagcacatttgaacgtt
gctggttgaaatcccgaggtgtcatttgacatggttctctgaacttatctttcctataaaatgg
tagttagatctggaggtctgattttgtggcaaaaatacttcctaggtggtgctgggtacttctt
gttgcatcctgtcaggaggcagataatgctggtgcctctctattggtaatgttaagactgctgg
gtgggtttggagttcttggctttaatcattcattacaaagttcagcatttta
SEQ ID NO: 14 = Ensembl polypeptide sequence of human CTSE
(396 amino acids)
MKTLLLLLLVLLELGEAQGSLHRVPLRRHPSLKKKLRARSQLSEFWKSHNLDMIQFTESCSMDQ
SAKEPLINYLDMEYFGTISIGSPPQNFTVIFDTGSSNLWVPSVYCTSPACKTHSRFQPSQSSTY
SQPGQSFSIQYGTGSLSGIIGADQVSVEGLTVVGQQFGESVTEPGQTFVDAEFDGILGLGYPSL
AVGGVTPVFDNMMAQNLVDLPMFSVYMSSNPEGGAGSELIFGGYDHSHFSGSLNWVPVTKQAYW
QIALDNIQVGGTVMFCSEGCQAIVDTGTSLITGPSDKIKQLQNAIGAAPVDGEYAVECANLNVM
PDVTFTINGVPYTLSPTAYTLLDFVDGMQFCSSGFQGLDIHPPAGPLWILGDVFIRQFYSVFDR
GNNRVGLAPAVP
SEQ ID NO: 15 = RefSeq nucleotide sequence encoding human TFF2 (mRNA)
cacggtggaagggctggggccacggggcagagaagaaaggttatctctgcttgttggacaaaca
gaggggagattataaaacatacccggcagtggacaccatgcattctgcaagccaccctggggtg
cagctgagctagacatgggacggcgagacgcccagctcctggcagcgctcctcgtcctggggct
atgtgccctggcggggagtgagaaaccctccccctgccagtgctccaggctgagcccccataac
aggacgaactgcggcttccctggaatcaccagtgaccagtgttttgacaatggatgctgtttcg
actccagtgtcactggggtcccctggtgtttccaccccctcccaaagcaagagtcggatcagtg
cgtcatggaggtctcagaccgaagaaactgtggctacccgggcatcagccccgaggaatgcgcc
tctcggaagtgctgcttctccaacttcatctttgaagtgccctggtgcttcttcccgaagtctg
tggaagactgccattactaagagaggctggttccagaggatgcatctggctcaccgggtgttcc
gaaaccaaagaagaaacttcgccttatcagcttcatacttcatgaaatcctgggttttcttaac
catcttttcctcattttcaatggtttaacatataatttctttaaataaaacccttaaaatctgc
taaaaaaaaaaaa
SEQ ID NO: 16 = RefSeq polypeptide sequence of human TFF2
(129 amino acids)
MGRRDAQLLAALLVLGLCALAGSEKPSPCQCSRLSPHNRTNCGFPGITSDQCFDNGCCFDSSVT
GVPWCFHPLPKQESDQCVMEVSDRRNCGYPGISPEECASRKCCFSNFIFEVPWCFFPKSVEDCH
Y
SEQ ID NO: 17 = Ensembl nucleotide sequence encoding human TFF2 (mRNA)
acagctgcctcttgcctcctcttcgcctccacggtggaagggctggggccacggggcagagaag
aaaggttatctctgcttgttggacaaacagaggggagattataaaacatacccggcagtggaca
ccatgcattctgcaagccaccctggggtgcagctgagctagacATGGGACGGCGAGACGCCCAG
CTCCTGGCAGCGCTCCTCGTCCTGGGGCTATGTGCCCTGGCGGGGAGTGAGAAACCCTCCCCCT
GCCAGTGCTCCAGGCTGAGCCCCCATAACAGGACGAACTGCGGCTTCCCTGGAATCACCAGTGA
CCAGTGTTTTGACAATGGATGCTGTTTCGACTCCAGTGTCACTGGGGTCCCCTGGTGTTTCCAC
CCCCTCCCAAAGCAAGAGTCGGATCAGTGCGTCATGGAGGTCTCAGACCGAAGAAACTGTGGCT
ACCCGGGCATCAGCCCCGAGGAATGCGCCTCTCGGAAGTGCTGCTTCTCCAACTTCATCTTTGA
AGTGCCCTGGTGCTTCTTCCCGAAGTCTGTGGAAGACTGCCATTACTAAgagaggctggttcca
gaggatgcatctggctcaccgggtgttccgaaaccaaagaagaaacttcgccttatcagcttca
tacttcatgaaatcctgggttttcttaaccatcttttcctcattttcaatggtttaacatataa
tttctttaaataaaacccttaaaatctgctaaa
SEQ ID NO: 18 = Ensembl polypeptide sequence of human TFF2
(129 amino acids)
MGRRDAQLLAALLVLGLCALAGSEKPSPCQCSRLSPHNRTNCGFPGITSDQCFDNGCCFDSSVT
GVPWCFHPLPKQESDQCVMEVSDRRNCGYPGISPEECASRKCCFSNFIFEVPWCFFPKSVEDCH
Y

1. A method of predicting the likelihood that a colorectal polyp in a subject will develop into colorectal cancer, the method comprising:
determining an expression level of at least one gene selected from MUC17, VSIG1, and CTSE in a sample obtained from the colorectal polyp;
comparing the expression level to a control value associated with that same gene; and
predicting the likelihood that the colorectal polyp will develop into colorectal cancer based on the relative difference between the expression level and the control value associated with each gene,
wherein an increase in the expression level at least one of MUC17, VSIG1, and CTSE relative to the control value associated with each gene correlates with an increased likelihood of the colorectal polyp developing into colorectal cancer.

2. The method of claim 1, the method further comprising:
determining an expression level of TFF2 in the sample obtained from the colorectal polyp,
wherein an increase in the expression level of TFF2 relative to the control value associated with TFF2 correlates with an increased likelihood of the colorectal polyp developing into colorectal cancer.

3. The method of claim 1, the method further comprising:
determining an expression level of at least one gene selected from TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, in a sample obtained from the colorectal polyp,
wherein an increase in the expression level at least one of TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, and ONECUT2 relative to the control value associated with each gene correlates with an increased likelihood of the colorectal polyp developing into colorectal cancer, and
wherein a decrease in the expression level at least one of SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1 relative to the control value associated with each gene correlates with an increased likelihood of the colorectal polyp developing into colorectal cancer.

4. The method of claim 1, further comprising determining the expression level of at least one gene selected from MUC5AC, KLK10, TFF1, DUOX2, CDH3, S100P, and GJB5 in the sample obtained from the colorectal polyp,
wherein an increase in the expression level of at least one of MUC5AC, KLK10, TFF1, DUOX2, CDH3, S100P, and GJB5 relative to the control value associated with the gene correlates with an increased likelihood of the colorectal polyp developing into colorectal cancer.

5. The method of claim 1, further comprising determining the expression level of at least one gene selected from SLC14A2, CD177, ZG16, and AQP8 in the sample obtained from the colorectal polyp,
wherein a decrease in the expression level of at least one of SLC14A2, CD177, ZG16, and AQP8 relative to the control value associated with the gene correlates with an increased likelihood of the colorectal polyp developing into colorectal cancer.

6. The method of claim 1, wherein when the expression level of at least one of MUC17, VSIG1, CTSE, TFF2, TM4SF4, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, MUC5AC, KLK10, TFF1, DUOX2, CDH3, S100P, and GJB5 is greater than the control value, the method further comprises diagnosing the polyp as being a sessile serrated adenoma/polyp.

7. The method of claim 6, further comprising diagnosing the subject as having serrated polyposis syndrome.

8. The method of claim 1, wherein when the control value is greater than the expression level of at least one of SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, TMIGD1, SLC14A2, CD177, ZG16, and AQP8, the method further comprises diagnosing the polyp as being a sessile serrated adenoma/polyp.

9. The method of claim 8, further comprising diagnosing the subject as having serrated polyposis syndrome.

10. The method of claim 1, wherein the control value associated with each gene is determined by determining the expression level of that gene in one or more control samples, and calculating an average expression level of that gene in the one or more control samples, wherein each control sample is obtained from healthy colonic tissue of the same or a different subject.

11. The method of claim 1, wherein determining the expression level of at least one gene comprises measuring the expression level of an RNA transcript of the at least one gene, or an expression product thereof.

12. The method of claim 11, wherein measuring the expression level of the RNA transcript of the at least one gene, or the expression product thereof, includes using at least one of a PCR-based method, a Northern blot method, a microarray method, and an immunohistochemical method.

13. The method of claim 1, comprising determining the expression level of at least three genes.

14. A method of determining the frequency of colonoscopies for a subject, the method comprising:
predicting the likelihood that a colorectal polyp in a subject will develop into colorectal cancer according to the method of claim 1,
wherein when there is an increased likelihood that the colorectal polyp will develop into colorectal cancer, increasing the frequency of colonoscopies administered to the subject.

15. A method of increasing the likelihood of detecting colorectal cancer at an early stage, the method comprising:
predicting the likelihood that a colorectal polyp in a subject will develop into colorectal cancer according to the method of claim 1,
wherein when there is an increased likelihood that the colorectal polyp will develop into colorectal cancer, increasing the frequency of colonoscopies administered to the subject.

16. A kit for predicting the likelihood that a colorectal polyp in a subject will develop into colorectal cancer, the kit comprising at least one primer, each adapted to amplify an RNA transcript of one gene independently selected from TM4SF4, VSIG1, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, and instructions for use.

17. The kit of claim 16, further comprising at least one additional primer, each adapted to amplify an RNA transcript of one gene independently selected from MUC5AC, KLK10, CTSE, TFF2, MUC17, TFF1, DUOX2, CDH3, S100P, GJB5, SLC14A2, CD177, ZG16, and AQP8.

18. A kit for predicting the likelihood that a colorectal polyp in a subject will develop into colorectal cancer, the kit comprising one or more probes, each adapted to specifically bind to an RNA transcript, or an expression product thereof, of one gene independently selected from TM4SF4, VSIG1, SERPINB5, KLK7, REG4, SLC6A14, ANXA10, HTR1D, KLK11, DUOXA2, VNN1, SULT1C2, AQP5, PI3, CLDN1, DUSP4, SLC6A20, TRIM29, PRSS22, TACSTD2, ST3GAL4, SDR16C5, ALDOB, HOXB13, KRT7, GJB4, APOB, PSCA, CIDEC, XKR9, DPCR1, RAB3B, FIBCD1, NXF3, PDZK1IP1, ZIC5, CEACAM18, CXCL1, MDFI, ONECUT2, SLC37A2, FAM3B, B4GALNT2, POPDC3, SLC30A10, PCDH20, UGT2A3, HSD3B2, CNTFR, EYA2, PITX2, G6PC, UGT1A4, PRKG2, ADH1C, CWH43, SLC17A8, MOCS1, NPY1R, TRIM9, and TMIGD1, and instructions for use.

19. The kit of claim 18, further comprising one or more additional probes, each adapted to specifically bind to an RNA transcript, or an expression product thereof, of one gene independently selected from MUC5AC, KLK10, CTSE, TFF2, MUC17, TFF1, DUOX2, CDH3, S100P, GJB5, SLC14A2, CD177, ZG16, and AQP8.

20. The kit of claim 18, wherein at least one probe comprises an antibody to an expression product.

21. The kit of claim 18, wherein at least one probe comprises an oligonucleotide complementary to an RNA transcript.


SEQUENCE LISTING (18 sequences)

SEQ ID NO: 1 - DNA, 20 bp, Artificial Sequence
  Synthetic Oligonucleotide
agggctccagcttgtatcac

SEQ ID NO: 2 - DNA, 20 bp, Artificial Sequence
  Synthetic Oligonucleotide
cgattcaaggagggttctga

SEQ ID NO: 3 - DNA, 14360 bp, Homo sapiens
tttcgccagctcctctgggggtgacaggcaagtgagacgtgctcagagctccgatgccaaggccagggaccatggcgctgtgtctgctgaccttggtcctctcgctcttgcccccacaagctgctgcagaacaggacctcagtgtgaacagggctgtgtgggatggaggagggtgcatctcccaaggggacgtcttgaaccgtcagtgccagcagctgtctcagcacgttaggacaggttctgcggcaaacaccgccacaggtacaacatctacaaatgtcgtggagccaagaatgtatttgagttgcagcaccaaccctgagatgacctcgattgagtccagtgtgacttcagacactcctggtgtctccagtaccaggatgacaccaacagaatccagaacaacttcagaatctaccagtgacagcaccacacttttccccagttctactgaagacacttcatctcctacaactcctgaaggcaccgacgtgcccatgtcaacaccaagtgaagaaagcatttcatcaacaatggcttttgtcagcactgcacctcttcccagttttgaggcctacacatctttaacatataaggttgatatgagcacacctctgaccacttctactcaggcaagttcatctcctactactcctgaaagcaccaccatacccaaatcaactaacagtgaaggaagcactccattaacaagtatgcctgccagcaccatgaaggtggccagttcagaggctatcacccttttgacaactcctgttgaaatcagcacacctgtgaccatttctgctcaagccagttcatctcctacaactgctgaaggtcccagcctgtcaaactcagctcctagtggaggaagcactccattaacaagaatgcctctcagcgtgatgctggtggtcagttctgaggctagcaccctttcaacaactcctgctgccaccaacattcctgtgatcacttctactgaagccagttcatctcctacaacggctgaaggcaccagcataccaacctcaacttatactgaaggaagcactccattaacaagtacgcctgccagcaccatgccggttgccacttctgaaatgagcacactttcaataactcctgttgacaccagcacacttgtgaccacttctactgaacccagttcacttcctacaactgctgaagctaccagcatgctaacctcaactcttagtgaaggaagcactccattaacaaatatgcctgtcagcaccatattggtggccagttctgaggctagcaccacttcaacaattcctgttgactccaaaacttttgtgaccactgctagtgaagccagctcatctcccacaactgctgaagataccagcattgcaacctcaactcctagtgaaggaagcactccattaacaagtatgcctgtcagcaccactccagtggccagttctgaggctagcaacctttcaacaactcctgttgactccaaaactcaggtgaccacttctactgaagccagttcatctcctccaactgctgaagttaacagcatgccaacctcaactcctagtgaaggaagcactccattaacaagtatgtctgtcagcaccatgccggtggccagttctgaggctagcaccctttcaacaactcctgttgacaccagcacacctgtgaccacttctagtgaagccagttcatcttctacaactcctgaaggtaccagcataccaacctcaactcctagtgaaggaagcactccattaacaaacatgcctgtcagcaccaggctggtggtcagttctgaggctagcaccacttcaacaactcctgctgactccaacacttttgtgaccacttctagtgaagctagttcatcttctacaactgctgaaggtaccagcatgccaacctcaacttacagtgaaagaggcactacaataacaagtatgtctgtcagcaccacactggtggccagttctgaggctagcaccctttcaacaactcctgttgactccaacactcctgtgaccacttcaactgaagccacttcatcttctacaactgcggaaggtaccagcatgccaacctcaacttatactgaaggaagcactccattaacaagtatgcctgtcaacaccacactggtggccagttctgaggctagcaccctttcaacaactcctgttgacaccagcacacctgtgaccacttcaactgaagccagttcctctcctacaactgctgatggtgccagtatgccaacctcaactcctagtgaaggaagcactccattaacaagtatgcctgtcagcaaaacgctgttgaccagttctgaggctagcaccctttcaacaactcctcttgacacaagcacacatatcaccacttctactgaagccagttgctctcctacaaccactgaaggtaccagcatgccaatctcaactcctagtgaaggaagtcctttattaacaagtatacctgtcagcatcacaccggtgaccagtcctgaggctagcaccctttcaacaactcctgttgactccaacagtcctgtgaccacttctactgaagtcagttcatctcctacacctgctgaaggtaccagcatgccaacctcaacttatagtgaaggaagaactcctttaacaagtatgcctgtcagcaccacactggtggccacttctgcaatcagcaccctttcaacaactcctgttgacaccagcacacctgtgaccaattctactgaagcccgttcgtctcctacaacttctgaaggtaccagcatgccaacctcaactcctggggaaggaagcactccattaacaagtatgcctgacagcaccacgccggtagtcagttctgaggctagaacactttcagcaactcctgttgacaccagcacacctgtgaccacttctactgaagccacttcatctcctacaactgctgaaggtaccagcataccaacctcgactcctagtgaaggaacgactccattaacaagcacacctgtcagccacacgctggtggccaattctgaggctagcaccctttcaacaactcctgttgactccaacactcctttgaccacttctactgaagccagttcacctcctcccactgctgaaggtaccagcatgccaacctcaactcctagtgaaggaagcactccattaacacgtatgcctgtcagcaccacaatggtggccagttctgaaacgagcacactttcaacaactcctgctgacaccagcacacctgtgaccacttattctcaagccagttcatcttctacaactgctgacggtaccagcatgccaacctcaacttatagtgaaggaagcactccactaacaagtgtgcctgtcagcaccaggctggtggtcagttctgaggctagcaccctttccacaactcctgtcgacaccagcatacctgtcaccacttctactgaagccagttcatctcctacaactgctgaaggtaccagcataccaacctcacctcccagtgaaggaaccactccgttagcaagtatgcctgtcagcaccacgctggtggtcagttctgaggctaacaccctttcaacaactcctgtggactccaaaactcaggtggccacttctactgaagccagttcacctcctccaactgctgaagttaccagcatgccaacctcaactcctggagaaagaagcactccattaacaagtatgcctgtcagacacacgccagtggccagttctgaggctagcaccctttcaacatctcccgttgacaccagcacacctgtgaccacttctgctgaaaccagttcctctcctacaaccgctgaaggtaccagcttgccaacctcaactactagtgaaggaagtactctattaacaagtatacctgtcagcaccacgctggtgaccagtcctgaggctagcacccttttaacaactcctgttgacactaaaggtcctgtggtcacttctaatgaagtcagttcatctcctacacctgctgaaggtaccagcatgccaacctcaacttatagtgaaggaagaactcctttaacaagtatacctgtcaacaccacactggtggccagttctgcaatcagcatcctttcaacaactcctgttgacaacagcacacctgtgaccacttctactgaagcctgttcatctcctacaacttctgaaggtaccagcatgccaaactcaaatcctagtgaaggaaccactccgttaacaagtatacctgtcagcaccacgccggtagtcagttctgaggctagcaccctttcagcaactcctgttgacaccagcacccctgggaccacttctgctgaagccacttcatctcctacaactgctgaaggtatcagcataccaacctcaactcctagtgaaggaaagactccattaaaaagtatacctgtcagcaacacgccggtggccaattctgaggctagcaccctttcaacaactcctgttgactctaacagtcctgtggtcacttctacagcagtcagttcatctcctacacctgctgaaggtaccagcatagcaatctcaacgcctagtgaaggaagcactgcattaacaagtatacctgtcagcaccacaacagtggccagttctgaaatcaacagcctttcaacaactcctgctgtcaccagcacacctgtgaccacttattctcaagccagttcatctcctacaactgctgacggtaccagcatgcaaacctcaacttatagtgaaggaagcactccactaacaagtttgcctgtcagcaccatgctggtggtcagttctgaggctaacaccctttcaacaacccctattgactccaaaactcaggtgaccgcttctactgaagccagttcatctacaaccgctgaaggtagcagcatgacaatctcaactcctagtgaaggaagtcctctattaacaagtatacctgtcagcaccacgccggtggccagtcctgaggctagcaccctttcaacaactcctgttgactccaacagtcctgtgatcacttctactgaagtcagttcatctcctacacctgctgaaggtaccagcatgccaacctcaacttatactgaaggaagaactcctttaacaagtataactgtcagaacaacaccggtggccagctctgcaatcagcaccctttcaacaactcccgttgacaacagcacacctgtgaccacttctactgaagcccgttcatctcctacaacttctgaaggtaccagcatgccaaactcaactcctagtgaaggaaccactccattaacaagtatacctgtcagcaccacgccggtactcagttctgaggctagcaccctttcagcaactcctattgacaccagcacccctgtgaccacttctactgaagccacttcgtctcctacaactgctgaaggtaccagcataccaacctcgactcttagtgaaggaatgactccattaacaagcacacctgtcagccacacgctggtggccaattctgaggctagcaccctttcaacaactcctgttgactctaacagtcctgtggtcacttctacagcagtcagttcatctcctacacctgctgaaggtaccagcatagcaacctcaacgcctagtgaaggaagcactgcattaacaagtatacctgtcagcaccacaacagtggccagttctgaaaccaacaccctttcaacaactcccgctgtcaccagcacacctgtgaccacttatgctcaagtcagttcatctcctacaactgctgacggtagcagcatgccaacctcaactcctagggaaggaaggcctccattaacaagtatacctgtcagcaccacaacagtggccagttctgaaatcaacaccctttcaacaactcttgctgacaccaggacacctgtgaccacttattctcaagccagttcatctcctacaactgctgatggtaccagcatgccaaccccagcttatagtgaaggaagcactccactaacaagtatgcctctcagcaccacgctggtggtcagttctgaggctagcactctttccacaactcctgttgacaccagcactcctgccaccacttctactgaaggcagttcatctcctacaactgcaggaggtaccagcatacaaacctcaactcctagtgaacggaccactccattagcaggtatgcctgtcagcactacgcttgtggtcagttctgagggtaacaccctttcaacaactcctgttgactccaaaactcaggtgaccaattctactgaagccagttcatctgcaaccgctgaaggtagcagcatgacaatctcagctcctagtgaaggaagtcctctactaacaagtatacctctcagcaccacgccggtggccagtcctgaggctagcaccctttcaacaactcctgttgactccaacagtcctgtgatcacttctactgaagtcagttcatctcctatacctactgaaggtaccagcatgcaaacctcaacttatagtgacagaagaactcctttaacaagtatgcctgtcagcaccacagtggtggccagttctgcaatcagcaccctttcaacaactcctgttgacaccagcacacctgtgaccaattctactgaagcccgttcatctcctacaacttctgaaggtaccagcatgccaacctcaactcctagtgaaggaagcactccattcacaagtatgcctgtcagcaccatgccggtagttacttctgaggctagcaccctttcagcaactcctgttgacaccagcacacctgtgaccacttctactgaagccacttcatctcctacaactgctgaaggtaccagcataccaacttcaactcttagtgaaggaacgactccattaacaagtatacctgtcagccacacgctggtggccaattctgaggttagcaccctttcaacaactcctgttgactccaacactcctttcactacttctactgaagccagttcacctcctcccactgctgaaggtaccagcatgccaacctcaacttctagtgaaggaaacactccattaacacgtatgcctgtcagcaccacaatggtggccagttttgaaacaagcacactttctacaactcctgctgacaccagcacacctgtgactacttattctcaagccggttcatctcctacaactgctgacgatactagcatgccaacctcaacttatagtgaaggaagcactccactaacaagtgtgcctgtcagcaccatgccggtggtcagttctgaggctagcacccattccacaactcctgttgacaccagcacacctgtcaccacttctactgaagccagttcatctcctacaactgctgaaggtaccagcataccaacctcacctcctagtgaaggaaccactccgttagcaagtatgcctgtcagcaccacgccggtggtcagttctgaggctggcaccctttccacaactcctgttgacaccagcacacctatgaccacttctactgaagccagttcatctcctacaactgctgaagatatcgtcgtgccaatctcaactgctagtgaaggaagtactctattaacaagtatacctgtcagcaccacgccagtggccagtcctgaggctagcaccctttcaacaactcctgttgactccaacagtcctgtggtcacttctactgaaatcagttcatctgctacatccgctgaaggtaccagcatgcctacctcaacttatagtgaaggaagcactccattaagaagtatgcctgtcagcaccaagccgttggccagttctgaggctagcactctttcaacaactcctgttgacaccagcatacctgtcaccacttctactgaaaccagttcatctcctacaactgcaaaagataccagcatgccaatctcaactcctagtgaagtaagtacttcattaacaagtatacttgtcagcaccatgccagtggccagttctgaggctagcaccctttcaacaactcctgttgacaccaggacacttgtgaccacttccactggaaccagttcatctcctacaactgctgaaggtagcagcatgccaacctcaactcctggtgaaagaagcactccattaacaaatatacttgtcagcaccacgctgttggccaattctgaggctagcaccctttcaacaactcctgttgacaccagcacacctgtcaccacttctgctgaagccagttcttctcctacaactgctgaaggtaccagcatgcgaatctcaactcctagtgatggaagtactccattaacaagtatacttgtcagcaccctgccagtggccagttctgaggctagcaccgtttcaacaactgctgttgacaccagcatacctgtcaccacttctactgaagccagttcctctcctacaactgctgaagttaccagcatgccaacctcaactcctagtgaaacaagtactccattaactagtatgcctgtcaaccacacgccagtggccagttctgaggctggcaccctttcaacaactcctgttgacaccagcacacctgtgaccacttctactaaagccagttcatctcctacaactgctgaaggtatcgtcgtgccaatctcaactgctagtgaaggaagtactctattaacaagtatacctgtcagcaccacgccggtggccagttctgaggctagcaccctttcaacaactcctgttgataccagcatacctgtcaccacttctactgaaggcagttcttctcctacaactgctgaaggtaccagcatgccaatctcaactcctagtgaagtaagtactccattaacaagtatacttgtcagcaccgtgccagtggccggttctgaggctagcaccctttcaacaactcctgttgacaccaggacacctgtcaccacttctgctgaagctagttcttctcctacaactgctgaaggtaccagcatgccaatctcaactcctggcgaaagaagaactccattaacaagtatgtctgtcagcaccatgccggtggccagttctgaggctagcaccctttcaagaactcctgctgacaccagcacacctgtgaccacttctactgaagccagttcctctcctacaactgctgaaggtaccggcataccaatctcaactcctagtgaaggaagtactccattaacaagtatacctgtcagcaccacgccagtggccattcctgaggctagcaccctttcaacaactcctgttgactccaacagtcctgtggtcacttctactgaagtcagttcatctcctacacctgctgaaggtaccagcatgccaatctcaacttatagtgaaggaagcactccattaacaggtgtgcctgtcagcaccacaccggtgaccagttctgcaatcagcaccctttcaacaactcctgttgacaccagcacacctgtgaccacttctactgaagcccattcatctcctacaacttctgaaggtaccagcatgccaacctcaactcctagtgaaggaagtactccattaacatatatgcctgtcagcaccatgctggtagtcagttctgaggatagcaccctttcagcaactcctgttgacaccagcacacctgtgaccacttctactgaagccacttcatctacaactgctgaaggtaccagcattccaacctcaactcctagtgaaggaatgactccattaactagtgtacctgtcagcaacacgccggtggccagttctgaggctagcatcctttcaacaactcctgttgactccaacactcctttgaccacttctactgaagccagttcatctcctcccactgctgaaggtaccagcatgccaacctcaactcctagtgaaggaagcactccattaacaagtatgcctgtcagcaccacaacggtggccagttctgaaacgagcaccctttcaacaactcctgctgacaccagcacacctgtgaccacttattctcaagccagttcatctcctccaattgctgacggtactagcatgccaacctcaacttatagtgaaggaagcactccactaacaaatatgtctttcagcaccacgccagtggtcagttctgaggctagcaccctttccacaactcctgttgacaccagcacacctgtcaccacttctactgaagccagtttatctcctacaactgctgaaggtaccagcataccaacctcaagtcctagtgaaggaaccactccattagcaagtatgcctgtcagcaccacgccggtggtcagttctgaggttaacaccctttcaacaactcctgtggactccaacactctggtgaccacttctactgaagccagttcatctcctacaatcgctgaaggtaccagcttgccaacctcaactactagtgaaggaagcactccattatcaattatgcctctcagtaccacgccggtggccagttctgaggctagcaccctttcaacaactcctgttgacaccagcacacctgtgaccacttcttctccaaccaattcatctcctacaactgctgaagttaccagcatgccaacatcaactgctggtgaaggaagcactccattaacaaatatgcctgtcagcaccacaccggtggccagttctgaggctagcaccctttcaacaactcctgttgactccaacacttttgttaccagttctagtcaagccagttcatctccagcaactcttcaggtcaccactatgcgtatgtctactccaagtgaaggaagctcttcattaacaactatgctcctcagcagcacatatgtgaccagttctgaggctagcacaccttccactccttctgttgacagaagcacacctgtgaccacttctactcagagcaattctactcctacacctcctgaagttatcaccctgccaatgtcaactcctagtgaagtaagcactccattaaccattatgcctgtcagcaccacatcggtgaccatttctgaggctggcacagcttcaacacttcctgttgacaccagcacacctgtgatcacttctacccaagtcagttcatctcctgtgactcctgaaggtaccaccatgccaatctggacgcctagtgaaggaagcactccattaacaactatgcctgtcagcaccacacgtgtgaccagctctgagggtagcaccctttcaacaccttctgttgtcaccagcacacctgtgaccacttctactgaagccatttcatcttctgcaactcttgacagcaccaccatgtctgtgtcaatgcccatggaaataagcacccttgggaccactattcttgtcagtaccacacctgttacgaggtttcctgagagtagcaccccttccataccatctgtttacaccagcatgtctatgaccactgcctctgaaggcagttcatctcctacaactcttgaaggcaccaccaccatgcctatgtcaactacgagtgaaagaagcactttattgacaactgtcctcatcagccctatatctgtgatgagtccttctgaggccagcacactttcaacacctcctggtgataccagcacacctttgctcacctctaccaaagccggttcattctccatacctgctgaagtcactaccatacgtatttcaattaccagtgaaagaagcactccattaacaactctccttgtcagcaccacacttccaactagctttcctggggccagcatagcttcgacacctcctcttgacacaagcacaacttttaccccttctactgacactgcctcaactcccacaattcctgtagccaccaccatatctgtatcagtgatcacagaaggaagcacacctgggacaaccatttttattcccagcactcctgtcaccagttctactgctgatgtctttcctgcaacaactggtgctgtatctacccctgtgataacttccactgaactaaacacaccatcaacctccagtagtagtaccaccacatctttttcaactactaaggaatttacaacacccgcaatgactactgcagctcccctcacatatgtgaccatgtctactgcccccagcacacccagaacaaccagcagaggctgcactacttctgcatcaacgctttctgcaaccagtacacctcacacctctacttctgtcaccacccgtcctgtgaccccttcatcagaatccagcaggccgtcaacaattacttctcacaccatcccacctacatttcctcctgctcactccagtacacctccaacaacctctgcctcctccacgactgtgaaccctgaggctgtcaccaccatgaccaccaggacaaaacccagcacacggaccacttccttccccacggtgaccaccaccgctgtccccacgaatactacaattaagagcaaccccacctcaactcctactgtgccaagaaccacaacatgctttggagatgggtgccagaatacggcctctcgctgcaagaatggaggcacctgggatgggctcaagtgccagtgtcccaacctctattatggggagttgtgtgaggaggtggtcagcagcattgacatagggccaccggagactatctctgcccaaatggaactgactgtgacagtgaccagtgtgaagttcaccgaagagctaaaaaaccactcttcccaggaattccaggagttcaaacagacattcacggaacagatgaatattgtgtattccgggatccctgagtatgtcggggtgaacatcacaaagctacgtcttggcagtgtggtggtggagcatgacgtcctcctaagaaccaagtacacaccagaatacaagacagtattggacaatgccaccgaagtagtgaaagagaaaatcacaaaagtgaccacacagcaaataatgattaatgatatttgctcagacatgatgtgtttcaacaccactggcacccaagtgcaaaacattacggtgacccagtacgaccctgaagaggactgccggaagatggccaaggaatatggagactacttcgtagtggagtaccgggaccagaagccatactgcatcagcccctgtgagcctggcttcagtgtctccaagaactgtaacctcggcaagtgccagatgtctctaagtggacctcagtgcctctgcgtgaccacggaaactcactggtacagtggggagacctgtaaccagggcacccagaagagtctggtgtacggcctcgtgggggcaggggtcgtgctgatgctgatcatcctggtagctctcctgatgctcgttttccgctccaagagagaggtgaaacggcaaaagtacagattgtctcagttatacaagtggcaagaagaggacagtggaccagctcctgggaccttccaaaacattggctttgacatctgccaagatgatgattccatccacctggagtccatctatagtaatttccagccctccttgagacacatagaccctgaaacaaagatccgaattcagaggcctcaggtaatgacgacatcattttaaggcatggagctgagaagtctgggagtgaggagatcccagtccggctaagcttggtggagcattttcccattgagagccttccatgggaactcaatgttcccattgtaagtacaggaaacaagccctgtacttaccaaggagaaagaggagagacagcagtgctgggagattctcaaatagaaacccgtggacgctccaatgggcttgtcatgatatcaggctaggctttcctgctcatttttcaaagacgctccagatttgagggtactctgactgcaacatctttcaccccattgatcgccaggattgatttggttgatctggctgagcaggcgggtgtccccgtcctccctcactgccccatatgtgtccctcctaaagctgcatgctcagttgaagaggacgagaggacgaccttctctgatagaggaggaccacgcttcagtcaaaggcatacaagtatctatctggacttccctgctagcacttccaaacaagctcagagatgttcctcccctcatctgcccgggttcagtaccatggacagcgccctcgacccgctgtttacaaccatgaccccttggacactggactgcatgcactttacatatcacaaaatgctctcataagaattattgcataccatcttcatgaaaaacacctgtatttaaatatagagcatttaccttttggtatataagattgtgggtattttttaagttcttattgttatgagttctgattttttccttagtaaatattataatatatatttgtagtaactaaaaataataaagcaattttattacaattttaaaaaaaaaa

SEQ ID NO: 4 - protein, 4493 aa, Homo sapiens
MPRPGTMALCLLTLVLSLLPPQAAAEQDLSVNRAVWDGGGCISQGDVLNRQCQQLSQHVRTGSAANTATGTTSTNVVEPRMYLSCSTNPEMTSIESSVTSDTPGVSSTRMTPTESRTTSESTSDSTTLFPSSTEDTSSPTTPEGTDVPMSTPSEESISSTMAFVSTAPLPSFEAYTSLTYKVDMSTPLTTSTQASSSPTTPESTTIPKSTNSEGSTPLTSMPASTMKVASSEAITLLTTPVEISTPVTISAQASSSPTTAEGPSLSNSAPSGGSTPLTRMPLSVMLVVSSEASTLSTTPAATNIPVITSTEASSSPTTAEGTSIPTSTYTEGSTPLTSTPASTMPVATSEMSTLSITPVDTSTLVTTSTEPSSLPTTAEATSMLTSTLSEGSTPLTNMPVSTILVASSEASTTSTIPVDSKTFVTTASEASSSPTTAEDTSIATSTPSEGSTPLTSMPVSTTPVASSEASNLSTTPVDSKTQVTTSTEASSSPPTAEVNSMPTSTPSEGSTPLTSMSVSTMPVASSEASTLSTTPVDTSTPVTTSSEASSSSTTPEGTSIPTSTPSEGSTPLTNMPVSTRLVVSSEASTTSTTPADSNTFVTTSSEASSSSTTAEGTSMPTSTYSERGTTITSMSVSTTLVASSEASTLSTTPVDSNTPVTTSTEATSSSTTAEGTSMPTSTYTEGSTPLTSMPVNTTLVASSEASTLSTTPVDTSTPVTTSTEASSSPTTADGASMPTSTPSEGSTPLTSMPVSKTLLTSSEASTLSTTPLDTSTHITTSTEASCSPTTTEGTSMPISTPSEGSPLLTSIPVSITPVTSPEASTLSTTPVDSNSPVTTSTEVSSSPTPAEGTSMPTSTYSEGRTPLTSMPVSTTLVATSAISTLSTTPVDTSTPVTNSTEARSSPTTSEGTSMPTSTPGEGSTPLTSMPDSTTPVVSSEARTLSATPVDTSTPVTTSTEATSSPTTAEGTSIPTSTPSEGTTPLTSTPVSHTLVANSEASTLSTTPVDSNTPLTTSTEASSPPPTAEGTSMPTSTPSEGSTPLTRMPVSTTMVASSETSTLSTTPADTSTPVTTYSQASSSSTTADGTSMPTSTYSEGSTPLTSVPVSTRLVVSSEASTLSTTPVDTSIPVTTSTEASSSPTTAEGTSIPTSPPSEGTTPLASMPVSTTLVVSSEANTLSTTPVDSKTQVATSTEASSPPPTAEVTSMPTSTPGERSTPLTSMPVRHTPVASSEASTLSTSPVDTSTPVTTSAETSSSPTTAEGTSLPTSTTSEGSTLLTSIPVSTTLVTSPEASTLLTTPVDTKGPVVTSNEVSSSPTPAEGTSMPTSTYSEGRTPLTSIPVNTTLVASSAISILSTTPVDNSTPVTTSTEACSSPTTSEGTSMPNSNPSEGTTPLTSIPVSTTPVVSSEASTLSATPVDTSTPGTTSAEATSSPTTAEGISIPTSTPSEGKTPLKSIPVSNTPVANSEASTLSTTPVDSNSPVVTSTAVSSSPTPAEGTSIAISTPSEGSTALTSIPVSTTTVASSEINSLSTTPAVTSTPVTTYSQASSSPTTADGTSMQTSTYSEGSTPLTSLPVSTMLVVSSEANTLSTTPIDSKTQVTASTEASSSTTAEGSSMTISTPSEGSPLLTSIPVSTTPVASPEASTLSTTPVDSNSPVITSTEVSSSPTPAEGTSMPTSTYTEGRTPLTSITVRTTPVASSAISTLSTTPVDNSTPVTTSTEARSSPTTSEGTSMPNSTPSEGTTPLTSIPVSTTPVLSSEASTLSATPIDTSTPVTTSTEATSSPTTAEGTSIPTSTLSEGMTPLTSTPVSHTLVANSEASTLSTTPVDSNSPVVTSTAVSSSPTPAEGTSIATSTPSEGSTALTSIPVSTTTVASSETNTLSTTPAVTSTPVTTYAQVSSSPTTADGSSMPTSTPREGRPPLTSIPVSTTTVASSEINTLSTTLADTRTPVTTYSQASSSPTTADGTSMPTPAYSEGSTPLTSMPLSTTLVVSSEASTLSTTPVDTSTPATTSTEGSSSPTTAGGTSIQTSTPSERTTPLAGMPVSTTLVVSSEGNTLSTTPVDSKTQVTNSTEASSSATAEGSSMTISAPSEGSPLLTSIPLSTTPVASPEASTLSTTPVDSNSPVITSTEVSSSPIPTEGTSMQTSTYSDRRTPLTSMPVSTTVVASSAISTLSTTPVDTSTPVTNSTEARSSPTTSEGTSMPTSTPSEGSTPFTSMPVSTMPVVTSEASTLSATPVDTSTPVTTSTEATSSPTTAEGTSIPTSTLSEGTTPLTSIPVSHTLVANSEVSTLSTTPVDSNTPFTTSTEASSPPPTAEGTSMPTSTSSEGNTPLTRMPVSTTMVASFETSTLSTTPADTSTPVTTYSQAGSSPTTADDTSMPTSTYSEGSTPLTSVPVSTMPVVSSEASTHSTTPVDTSTPVTTSTEASSSPTTAEGTSIPTSPPSEGTTPLASMPVSTTPVVSSEAGTLSTTPVDTSTPMTTSTEASSSPTTAEDIVVPISTASEGSTLLTSIPVSTTPVASPEASTLSTTPVDSNSPVVTSTEISSSATSAEGTSMPTSTYSEGSTPLRSMPVSTKPLASSEASTLSTTPVDTSIPVTTSTETSSSPTTAKDTSMPISTPSEVSTSLTSILVSTMPVASSEASTLSTTPVDTRTLVTTSTGTSSSPTTAEGSSMPTSTPGERSTPLTNILVSTTLLANSEASTLSTTPVDTSTPVTTSAEASSSPTTAEGTSMRISTPSDGSTPLTSILVSTLPVASSEASTVSTTAVDTSIPVTTSTEASSSPTTAEVTSMPTSTPSETSTPLTSMPVNHTPVASSEAGTLSTTPVDTSTPVTTSTKASSSPTTAEGIVVPISTASEGSTLLTSIPVSTTPVASSEASTLSTTPVDTSIPVTTSTEGSSSPTTAEGTSMPISTPSEVSTPLTSILVSTVPVAGSEASTLSTTPVDTRTPVTTSAEASSSPTTAEGTSMPISTPGERRTPLTSMSVSTMPVASSEASTLSRTPADTSTPVTTSTEASSSPTTAEGTGIPISTPSEGSTPLTSIPVSTTPVAIPEASTLSTTPVDSNSPVVTSTEVSSSPTPAEGTSMPISTYSEGSTPLTGVPVSTTPVTSSAISTLSTTPVDTSTPVTTSTEAHSSPTTSEGTSMPTSTPSEGSTPLTYMPVSTMLVVSSEDSTLSATPVDTSTPVTTSTEATSSTTAEGTSIPTSTPSEGMTPLTSVPVSNTPVASSEASILSTTPVDSNTPLTTSTEASSSPPTAEGTSMPTSTPSEGSTPLTSMPVSTTTVASSETSTLSTTPADTSTPVTTYSQASSSPPIADGTSMPTSTYSEGSTPLTNMSFSTTPVVSSEASTLSTTPVDTSTPVTTSTEASLSPTTAEGTSIPTSSPSEGTTPLASMPVSTTPVVSSEVNTLSTTPVDSNTLVTTSTEASSSPTIAEGTSLPTSTTSEGSTPLSIMPLSTTPVASSEASTLSTTPVDTSTPVTTSSPTNSSPTTAEVTSMPTSTAGEGSTPLTNMPVSTTPVASSEASTLSTTPVDSNTFVTSSSQASSSPATLQVTTMRMSTPSEGSSSLTTMLLSSTYVTSSEASTPSTPSVDRSTPVTTSTQSNSTPTPPEVITLPMSTPSEVSTPLTIMPVSTTSVTISEAGTASTLPVDTSTPVITSTQVSSSPVTPEGTTMPIWTPSEGSTPLTTMPVSTTRVTSSEGSTLSTPSVVTSTPVTTSTEAISSSATLDSTTMSVSMPMEISTLGTTILVSTTPVTRFPESSTPSIPSVYTSMSMTTASEGSSSPTTLEGTTTMPMSTTSERSTLLTTVLISPISVMSPSEASTLSTPPGDTSTPLLTSTKAGSFSIPAEVTTIRISITSERSTPLTTLLVSTTLPTSFPGASIASTPPLDTSTTFTPSTDTASTPTIPVATTISVSVITEGSTPGTTIFIPSTPVTSSTADVFPATTGAVSTPVITSTELNTPSTSSSSTTTSFSTTKEFTTPAMTTAAPLTYVTMSTAPSTPRTTSRGCTTSASTLSATSTPHTSTSVTTRPVTPSSESSRPSTITSHTIPPTFPPAHSSTPPTTSASSTTVNPEAVTTMTTRTKPSTRTTSFPTVTTTAVPTNTTIKSNPTSTPTVPRTTTCFGDGCQNTASRCKNGGTWDGLKCQCPNLYYGELCEEVVSSIDIGPPETISAQMELTVTVTSVKFTEELKNHSSQEFQEFKQTFTEQMNIVYSGIPEYVGVNITKLRLGSVVVEHDVLLRTKYTPEYKTVLDNATEVVKEKITKVTTQQIMINDICSDMMCFNTTGTQVQNITVTQYDPEEDCRKMAKEYGDYFVVEYRDQKPYCISPCEPGFSVSKNCNLGKCQMSLSGPQCLCVTTETHWYSGETCNQGTQKSLVYGLVGAGVVLMLIILVALLMLVFRSKREVKRQKYRLSQLYKWQEEDSGPAPGTFQNIGFDICQDDDSIHLESIYSNFQPSLRHIDPETKIRIQRPQVMTTSF

SEQ ID NO: 5 - DNA, 14089 bp, Homo sapiens
tctgaggctcatttcgccagctcctctgggggtgacaggcaagtgagacgtgctcagagctccgatgccaaggccagggaccatggcgctgtgtctgctgaccttggtcctctcgctcttgcccccacaagctgctgcagaacaggacctcagtgtgaacagggctgtgtgggatggaggagggtgcatctcccaaggggacgtcttgaaccgtcagtgccagcagctgtctcagcacgttaggacaggttctgcggcaaacaccgccacaggtacaacatctacaaatgtcgtggagccaagaatgtatttgagttgcagcaccaaccctgagatgacctcgattgagtccagtgtgacttcagacactcctggtgtctccagtaccaggatgacaccaacagaatccagaacaacttcagaatctaccagtgacagcaccacacttttccccagttctactgaagacacttcatctcctacaactcctgaaggcaccgacgtgcccatgtcaacaccaagtgaagaaagcatttcatcaacaatggcttttgtcagcactgcacctcttcccagttttgaggcctacacatctttaacatataaggttgatatgagcacacctctgaccacttctactcaggcaagttcatctcctactactcctgaaagcaccaccatacccaaatcaactaacagtgaaggaagcactccattaacaagtatgcctgccagcaccatgaaggtggccagttcagaggctatcacccttttgacaactcctgttgaaatcagcacacctgtgaccatttctgctcaagccagttcatctcctacaactgctgaaggtcccagcctgtcaaactcagctcctagtggaggaagcactccattaacaagaatgcctctcagcgtgatgctggtggtcagttctgaggctagcaccctttcaacaactcctgctgccaccaacattcctgtgatcacttctactgaagccagttcatctcctacaacggctgaaggcaccagcataccaacctcaacttatactgaaggaagcactccattaacaagtacgcctgccagcaccatgccggttgccacttctgaaatgagcacactttcaataactcctgttgacaccagcacacttgtgaccacttctactgaacccagttcacttcctacaactgctgaagctaccagcatgctaacctcaactcttagtgaaggaagcactccattaacaaatatgcctgtcagcaccatattggtggccagttctgaggctagcaccacttcaacaattcctgttgactccaaaacttttgtgaccactgctagtgaagccagctcatctcccacaactgctgaagataccagcattgcaacctcaactcctagtgaaggaagcactccattaacaagtatgcctgtcagcaccactccagtggccagttctgaggctagcaacctttcaacaactcctgttgactccaaaactcaggtgaccacttctactgaagccagttcatctcctccaactgctgaagttaacagcatgccaacctcaactcctagtgaaggaagcactccattaacaagtatgtctgtcagcaccatgccggtggccagttctgaggctagcaccctttcaacaactcctgttgacaccagcacacctgtgaccacttctagtgaagccagttcatcttctacaactcctgaaggtaccagcataccaacctcaactcctagtgaaggaagcactccattaacaaacatgcctgtcagcaccaggctggtggtcagttctgaggctagcaccacttcaacaactcctgctgactccaacacttttgtgaccacttctagtgaagctagttcatcttctacaactgctgaaggtaccagcatgccaacctcaacttacagtgaaagaggcactacaataacaagtatgtctgtcagcaccacactggtggccagttctgaggctagcaccctttcaacaactcctgttgactccaacactcctgtgaccacttcaactgaagccacttcatcttctacaactgcggaaggtaccagcatgccaacctcaacttatactgaaggaagcactccattaacaagtatgcctgtcaacaccacactggtggccagttctgaggctagcaccctttcaacaactcctgttgacaccagcacacctgtgaccacttcaactgaagccagttcctctcctacaactgctgatggtgccagtatgccaacctcaactcctagtgaaggaagcactccattaacaagtatgcctgtcagcaaaacgctgttgaccagttctgaggctagcaccctttcaacaactcctcttgacacaagcacacatatcaccacttctactgaagccagttgctctcctacaaccactgaaggtaccagcatgccaatctcaactcctagtgaaggaagtcctttattaacaagtatacctgtcagcatcacaccggtgaccagtcctgaggctagcaccctttcaacaactcctgttgactccaacagtcctgtgaccacttctactgaagtcagttcatctcctacacctgctgaaggtaccagcatgccaacctcaacttatagtgaaggaagaactcctttaacaagtatgcctgtcagcaccacactggtggccacttctgcaatcagcaccctttcaacaactcctgttgacaccagcacacctgtgaccaattctactgaagcccgttcgtctcctacaacttctgaaggtaccagcatgccaacctcaactcctggggaaggaagcactccattaacaagtatgcctgacagcaccacgccggtagtcagttctgaggctagaacactttcagcaactcctgttgacaccagcacacctgtgaccacttctactgaagccacttcatctcctacaactgctgaaggtaccagcataccaacctcgactcctagtgaaggaacgactccattaacaagcacacctgtcagccacacgctggtggccaattctgaggctagcaccctttcaacaactcctgttgactccaacactcctttgaccacttctactgaagccagttcacctcctcccactgctgaaggtaccagcatgccaacctcaactcctagtgaaggaagcactccattaacacgtatgcctgtcagcaccacaatggtggccagttctgaaacgagcacactttcaacaactcctgctgacaccagcacacctgtgaccacttattctcaagccagttcatcttctacaactgctgacggtaccagcatgccaacctcaacttatagtgaaggaagcactccactaacaagtgtgcctgtcagcaccaggctggtggtcagttctgaggctagcaccctttccacaactcctgtcgacaccagcatacctgtcaccacttctactgaagccagttcatctcctacaactgctgaaggtaccagcataccaacctcacctcccagtgaaggaaccactccgttagcaagtatgcctgtcagcaccacgctggtggtcagttctgaggctaacaccctttcaacaactcctgtggactccaaaactcaggtggccacttctactgaagccagttcacctcctccaactgctgaagttaccagcatgccaacctcaactcctggagaaagaagcactccattaacaagtatgcctgtcagacacacgccagtggccagttctgaggctagcaccctttcaacatctcccgttgacaccagcacacctgtgaccacttctgctgaaaccagttcctctcctacaaccgctgaaggtaccagcttgccaacctcaactactagtgaaggaagtactctattaacaagtatacctgtcagcaccacgctggtgaccagtcctgaggctagcacccttttaacaactcctgttgacactaaaggtcctgtggtcacttctaatgaagtcagttcatctcctacacctgctgaaggtaccagcatgccaacctcaacttatagtgaaggaagaactcctttaacaagtatacctgtcaacaccacactggtggccagttctgcaatcagcatcctttcaacaactcctgttgacaacagcacacctgtgaccacttctactgaagcctgttcatctcctacaacttctgaaggtaccagcatgccaaactcaaatcctagtgaaggaaccactccgttaacaagtatacctgtcagcaccacgccggtagtcagttctgaggctagcaccctttcagcaactcctgttgacaccagcacccctgggaccacttctgctgaagccacttcatctcctacaactgctgaaggtatcagcataccaacctcaactcctagtgaaggaaagactccattaaaaagtatacctgtcagcaacacgccggtggccaattctgaggctagcaccctttcaacaactcctgttgactctaacagtcctgtggtcacttctacagcagtcagttcatctcctacacctgctgaaggtaccagcatagcaatctcaacgcctagtgaaggaagcactgcattaacaagtatacctgtcagcaccacaacagtggccagttctgaaatcaacagcctttcaacaactcctgctgtcaccagcacacctgtgaccacttattctcaagccagttcatctcctacaactgctgacggtaccagcatgcaaacctcaacttatagtgaaggaagcactccactaacaagtttgcctgtcagcaccatgctggtggtcagttctgaggctaacaccctttcaacaacccctattgactccaaaactcaggtgaccgcttctactgaagccagttcatctacaaccgctgaaggtagcagcatgacaatctcaactcctagtgaaggaagtcctctattaacaagtatacctgtcagcaccacgccggtggccagtcctgaggctagcaccctttcaacaactcctgttgactccaacagtcctgtgatcacttctactgaagtcagttcatctcctacacctgctgaaggtaccagcatgccaacctcaacttatactgaaggaagaactcctttaacaagtataactgtcagaacaacaccggtggccagctctgcaatcagcaccctttcaacaactcccgttgacaacagcacacctgtgaccacttctactgaagcccgttcatctcctacaacttctgaaggtaccagcatgccaaactcaactcctagtgaaggaaccactccattaacaagtatacctgtcagcaccacgccggtactcagttctgaggctagcaccctttcagcaactcctattgacaccagcacccctgtgaccacttctactgaagccacttcgtctcctacaactgctgaaggtaccagcataccaacctcgactcttagtgaaggaatgactccattaacaagcacacctgtcagccacacgctggtggccaattctgaggctagcaccctttcaacaactcctgttgactctaacagtcctgtggtcacttctacagcagtcagttcatctcctacacctgctgaaggtaccagcatagcaacctcaacgcctagtgaaggaagcactgcattaacaagtatacctgtcagcaccacaacagtggccagttctgaaaccaacaccctttcaacaactcccgctgtcaccagcacacctgtgaccacttatgctcaagtcagttcatctcctacaactgctgacggtagcagcatgccaacctcaactcctagggaaggaaggcctccattaacaagtatacctgtcagcaccacaacagtggccagttctgaaatcaacaccctttcaacaactcttgctgacaccaggacacctgtgaccacttattctcaagccagttcatctcctacaactgctgatggtaccagcatgccaaccccagcttatagtgaaggaagcactccactaacaagtatgcctctcagcaccacgctggtggtcagttctgaggctagcactctttccacaactcctgttgacaccagcactcctgccaccacttctactgaaggcagttcatctcctacaactgcaggaggtaccagcatacaaacctcaactcctagtgaacggaccactccattagcaggtatgcctgtcagcactacgcttgtggtcagttctgagggtaacaccctttcaacaactcctgttgactccaaaactcaggtgaccaattctactgaagccagttcatctgcaaccgctgaaggtagcagcatgacaatctcagctcctagtgaaggaagtcctctactaacaagtatacctctcagcaccacgccggtggccagtcctgaggctagcaccctttcaacaactcctgttgactccaacagtcctgtgatcacttctactgaagtcagttcatctcctatacctactgaaggtaccagcatgcaaacctcaacttatagtgacagaagaactcctttaacaagtatgcctgtcagcaccacagtggtggccagttctgcaatcagcaccctttcaacaactcctgttgacaccagcacacctgtgaccaattctactgaagcccgttcatctcctacaacttctgaaggtaccagcatgccaacctcaactcctagtgaaggaagcactccattcacaagtatgcctgtcagcaccatgccggtagttacttctgaggctagcaccctttcagcaactcctgttgacaccagcacacctgtgaccacttctactgaagccacttcatctcctacaactgctgaaggtaccagcataccaacttcaactcttagtgaaggaacgactccattaacaagtatacctgtcagccacacgctggtggccaattctgaggttagcaccctttcaacaactcctgttgactccaacactcctttcactacttctactgaagccagttcacctcctcccactgctgaaggtaccagcatgccaacctcaacttctagtgaaggaaacactccattaacacgtatgcctgtcagcaccacaatggtggccagttttgaaacaagcacactttctacaactcctgctgacaccagcacacctgtgactacttattctcaagccggttcatctcctacaactgctgacgatactagcatgccaacctcaacttatagtgaaggaagcactccactaacaagtgtgcctgtcagcaccatgccggtggtcagttctgaggctagcacccattccacaactcctgttgacaccagcacacctgtcaccacttctactgaagccagttcatctcctacaactgctgaaggtaccagcataccaacctcacctcctagtgaaggaaccactccgttagcaagtatgcctgtcagcaccacgccggtggtcagttctgaggctggcaccctttccacaactcctgttgacaccagcacacctatgaccacttctactgaagccagttcatctcctacaactgctgaagatatcgtcgtgccaatctcaactgctagtgaaggaagtactctattaacaagtatacctgtcagcaccacgccagtggccagtcctgaggctagcaccctttcaacaactcctgttgactccaacagtcctgtggtcacttctactgaaatcagttcatctgctacatccgctgaaggtaccagcatgcctacctcaacttatagtgaaggaagcactccattaagaagtatgcctgtcagcaccaagccgttggccagttctgaggctagcactctttcaacaactcctgttgacaccagcatacctgtcaccacttctactgaaaccagttcatctcctacaactgcaaaagataccagcatgccaatctcaactcctagtgaagtaagtacttcattaacaagtatacttgtcagcaccatgccagtggccagttctgaggctagcaccctttcaacaactcctgttgacaccaggacacttgtgaccacttccactggaaccagttcatctcctacaactgctgaaggtagcagcatgccaacctcaactcctggtgaaagaagcactccattaacaaatatacttgtcagcaccacgctgttggccaattctgaggctagcaccctttcaacaactcctgttgacaccagcacacctgtcaccacttctgctgaagccagttcttctcctacaactgctgaaggtaccagcatgcgaatctcaactcctagtgatggaagtactccattaacaagtatacttgtcagcaccctgccagtggccagttctgaggctagcaccgtttcaacaactgctgttgacaccagcatacctgtcaccacttctactgaagccagttcctctcctacaactgctgaagttaccagcatgccaacctcaactcctagtgaaacaagtactccattaactagtatgcctgtcaaccacacgccagtggccagttctgaggctggcaccctttcaacaactcctgttgacaccagcacacctgtgaccacttctactaaagccagttcatctcctacaactgctgaaggtatcgtcgtgccaatctcaactgctagtgaaggaagtactctattaacaagtatacctgtcagcaccacgccggtggccagttctgaggctagcaccctttcaacaactcctgttgataccagcatacctgtcaccacttctactgaaggcagttcttctcctacaactgctgaaggtaccagcatgccaatctcaactcctagtgaagtaagtactccattaacaagtatacttgtcagcaccgtgccagtggccggttctgaggctagcaccctttcaacaactcctgttgacaccaggacacctgtcaccacttctgctgaagctagttcttctcctacaactgctgaaggtaccagcatgccaatctcaactcctggcgaaagaagaactccattaacaagtatgtctgtcagcaccatgccggtggccagttctgaggctagcaccctttcaagaactcctgctgacaccagcacacctgtgaccacttctactgaagccagttcctctcctacaactgctgaaggtaccggcataccaatctcaactcctagtgaaggaagtactccattaacaagtatacctgtcagcaccacgccagtggccattcctgaggctagcaccctttcaacaactcctgttgactccaacagtcctgtggtcacttctactgaagtcagttcatctcctacacctgctgaaggtaccagcatgccaatctcaacttatagtgaaggaagcactccattaacaggtgtgcctgtcagcaccacaccggtgaccagttctgcaatcagcaccctttcaacaactcctgttgacaccagcacacctgtgaccacttctactgaagcccattcatctcctacaacttctgaaggtaccagcatgccaacctcaactcctagtgaaggaagtactccattaacatatatgcctgtcagcaccatgctggtagtcagttctgaggatagcaccctttcagcaactcctgttgacaccagcacacctgtgaccacttctactgaagccacttcatctacaactgctgaaggtaccagcattccaacctcaactcctagtgaaggaatgactccattaactagtgtacctgtcagcaacacgccggtggccagttctgaggctagcatcctttcaacaactcctgttgactccaacactcctttgaccacttctactgaagccagttcatctcctcccactgctgaaggtaccagcatgccaacctcaactcctagtgaaggaagcactccattaacaagtatgcctgtcagcaccacaacggtggccagttctgaaacgagcaccctttcaacaactcctgctgacaccagcacacctgtgaccacttattctcaagccagttcatctcctccaattgctgacggtactagcatgccaacctcaacttatagtgaaggaagcactccactaacaaatatgtctttcagcaccacgccagtggtcagttctgaggctagcaccctttccacaactcctgttgacaccagcacacctgtcaccacttctactgaagccagtttatctcctacaactgctgaaggtaccagcataccaacctcaagtcctagtgaaggaaccactccattagcaagtatgcctgtcagcaccacgccggtggtcagttctgaggttaacaccctttcaacaactcctgtggactccaacactctggtgaccacttctactgaagccagttcatctcctacaatcgctgaaggtaccagcttgccaacctcaactactagtgaaggaagcactccattatcaattatgcctctcagtaccacgccggtggccagttctgaggctagcaccctttcaacaactcctgttgacaccagcacacctgtgaccacttcttctccaaccaattcatctcctacaactgctgaagttaccagcatgccaacatcaactgctggtgaaggaagcactccattaacaaatatgcctgtcagcaccacaccggtggccagttctgaggctagcaccctttcaacaactcctgttgactccaacacttttgttaccagttctagtcaagccagttcatctccagcaactcttcaggtcaccactatgcgtatgtctactccaagtgaaggaagctcttcattaacaactatgctcctcagcagcacatatgtgaccagttctgaggctagcacaccttccactccttctgttgacagaagcacacctgtgaccacttctactcagagcaattctactcctacacctcctgaagttatcaccctgccaatgtcaactcctagtgaagtaagcactccattaaccattatgcctgtcagcaccacatcggtgaccatttctgaggctggcacagcttcaacacttcctgttgacaccagcacacctgtgatcacttctacccaagtcagttcatctcctgtgactcctgaaggtaccaccatgccaatctggacgcctagtgaaggaagcactccattaacaactatgcctgtcagcaccacacgtgtgaccagctctgagggtagcaccctttcaacaccttctgttgtcaccagcacacctgtgaccacttctactgaagccatttcatcttctgcaactcttgacagcaccaccatgtctgtgtcaatgcccatggaaataagcacccttgggaccactattcttgtcagtaccacacctgttacgaggtttcctgagagtagcaccccttccataccatctgtttacaccagcatgtctatgaccactgcctctgaaggcagttcatctcctacaactcttgaaggcaccaccaccatgcctatgtcaactacgagtgaaagaagcactttattgacaactgtcctcatcagccctatatctgtgatgagtccttctgaggccagcacactttcaacacctcctggtgataccagcacacctttgctcacctctaccaaagccggttcattctccatacctgctgaagtcactaccatacgtatttcaattaccagtgaaagaagcactccattaacaactctccttgtcagcaccacacttccaactagctttcctggggccagcatagcttcgacacctcctcttgacacaagcacaacttttaccccttctactgacactgcctcaactcccacaattcctgtagccaccaccatatctgtatcagtgatcacagaaggaagcacacctgggacaaccatttttattcccagcactcctgtcaccagttctactgctgatgtctttcctgcaacaactggtgctgtatctacccctgtgataacttccactgaactaaacacaccatcaacctccagtagtagtaccaccacatctttttcaactactaaggaatttacaacacccgcaatgactactgcagctcccctcacatatgtgaccatgtctactgcccccagcacacccagaacaaccagcagaggctgcactacttctgcatcaacgctttctgcaaccagtacacctcacacctctacttctgtcaccacccgtcctgtgaccccttcatcagaatccagcaggccgtcaacaattacttctcacaccatcccacctacatttcctcctgctcactccagtacacctccaacaacctctgcctcctccacgactgtgaaccctgaggctgtcaccaccatgaccaccaggacaaaacccagcacacggaccacttccttccccacggtgaccaccaccgctgtccccacgaatactacaattaagagcaaccccacctcaactcctactgtgccaagaaccacaacatgctttggagatgggtgccagaatacggcctctcgctgcaagaatggaggcacctgggatgggctcaagtgccagtgtcccaacctctattatggggagttgtgtgaggaggtggtcagcagcattgacatagggccaccggagactatctctgcccaaatggaactgactgtgacagtgaccagtgtgaagttcaccgaagagctaaaaaaccactcttcccaggaattccaggagttcaaacagacattcacggaacagatgaatattgtgtattccgggatccctgagtatgtcggggtgaacatcacaaagctacgacatgatgtgtttcaacaccactggcacccaagtgcaaaacattacggtgacccagtacgaccctgaagaggactgccggaagatggccaaggaatatggagactacttcgtagtggagtaccgggaccagaagccatactgcatcagcccctgtgagcctggcttcagtgtctccaagaactgtaacctcggcaagtgccagatgtctctaagtggacctcagtgcctctgcgtgaccacggaaactcactggtacagtggggagacctgtaaccagggcacccagaagagtctggtgtacggcctcgtgggggcaggggtcgtgctgatgctgatcatcctggtagctctcctgatgctcgttttccgctccaagagagaggtgaaacggcaaaagtacagattgtctcagttatacaagtggcaagaagaggacagtggaccagctcctgggaccttccaaaacattggctttgacatctgccaagatgatgattccatccacctggagtccatctatagtaatttccagccctccttgagacacatagaccctgaaacaaagatccgaattcagaggcctcaggtaatgacgacatcattttaaggcatggagctgagaagtctgggagtgaggagatcccagtccggctaagcttggtggagcattttcccattgagagccttccatgggaactcaatgttcccattgtaagtacaggaaacaagccctgtacttaccaaggagaaagaggagagacagcagtgctgggagattctcaaatagaaacccgtggacgctccaatgggcttgtcatgatatcaggctaggctttcctgctcatttttcaaagacgctccagatttgagggtactctgactgcaacatctttcaccccattgatcgccaggattgatttggttgatctggctgagcaggcgggtgtccccgtcctccctcactgccccatatgtgtccctcctaaagctgcatgctcagttgaagaggacgagaggacgaccttctctgatagaggaggaccacgcttcagtcaaaggcatacaagtatctatctggacttccctgctagcacttccaaacaagctcagagatgttcctcccctcatctgcccgggttcagtaccatggacagcgccctcgacccgctgtttacaaccatgaccccttggacactggactgcatgcactttacatatcacaaaatgctctcataagaattattgcataccatcttcatgaaaaacacctgtatttaaatatagagcatttaccttttggta

SEQ ID NO: 6 - protein, 4262 aa, Homo sapiens
MPRPGTMALCLLTLVLSLLPPQAAAEQDLSVNRAVWDGGGCISQGDVLNRQCQQLSQHVRTGSAANTATGTTSTNVVEPRMYLSCSTNPEMTSIESSVTSDTPGVSSTRMTPTESRTTSESTSDSTTLFPSSTEDTSSPTTPEGTDVPMSTPSEESISSTMAFVSTAPLPSFEAYTSLTYKVDMSTPLTTSTQASSSPTTPESTTIPKSTNSEGSTPLTSMPASTMKVASSEAITLLTTPVEISTPVTISAQASSSPTTAEGPSLSNSAPSGGSTPLTRMPLSVMLVVSSEASTLSTTPAATNIPVITSTEASSSPTTAEGTSIPTSTYTEGSTPLTSTPASTMPVATSEMSTLSITPVDTSTLVTTSTEPSSLPTTAEATSMLTSTLSEGSTPLTNMPVSTILVASSEASTTSTIPVDSKTFVTTASEASSSPTTAEDTSIATSTPSEGSTPLTSMPVSTTPVASSEASNLSTTPVDSKTQVTTSTEASSSPPTAEVNSMPTSTPSEGSTPLTSMSVSTMPVASSEASTLSTTPVDTSTPVTTSSEASSSSTTPEGTSIPTSTPSEGSTPLTNMPVSTRLVVSSEASTTSTTPADSNTFVTTSSEASSSSTTAEGTSMPTSTYSERGTTITSMSVSTTLVASSEASTLSTTPVDSNTPVTTSTEATSSSTTAEGTSMPTSTYTEGSTPLTSMPVNTTLVASSEASTLSTTPVDTSTPVTTSTEASSSPTTADGASMPTSTPSEGSTPLTSMPVSKTLLTSSEASTLSTTPLDTSTHITTSTEASCSPTTTEGTSMPISTPSEGSPLLTSIPVSITPVTSPEASTLSTTPVDSNSPVTTSTEVSSSPTPAEGTSMPTSTYSEGRTPLTSMPVSTTLVATSAISTLSTTPVDTSTPVTNSTEARSSPTTSEGTSMPTSTPGEGSTPLTSMPDSTTPVVSSEARTLSATPVDTSTPVTTSTEATSSPTTAEGTSIPTSTPSEGTTPLTSTPVSHTLVANSEASTLSTTPVDSNTPLTTSTEASSPPPTAEGTSMPTSTPSEGSTPLTRMPVSTTMVASSETSTLSTTPADTSTPVTTYSQASSSSTTADGTSMPTSTYSEGSTPLTSVPVSTRLVVSSEASTLSTTPVDTSIPVTTSTEASSSPTTAEGTSIPTSPPSEGTTPLASMPVSTTLVVSSEANTLSTTPVDSKTQVATSTEASSPPPTAEVTSMPTSTPGERSTPLTSMPVRHTPVASSEASTLSTSPVDTSTPVTTSAETSSSPTTAEGTSLPTSTTSEGSTLLTSIPVSTTLVTSPEASTLLTTPVDTKGPVVTSNEVSSSPTPAEGTSMPTSTYSEGRTPLTSIPVNTTLVASSAISILSTTPVDNSTPVTTSTEACSSPTTSEGTSMPNSNPSEGTTPLTSIPVSTTPVVSSEASTLSATPVDTSTPGTTSAEATSSPTTAEGISIPTSTPSEGKTPLKSIPVSNTPVANSEASTLSTTPVDSNSPVVTSTAVSSSPTPAEGTSIAISTPSEGSTALTSIPVSTTTVASSEINSLSTTPAVTSTPVTTYSQASSSPTTADGTSMQTSTYSEGSTPLTSLPVSTMLVVSSEANTLSTTPIDSKTQVTASTEASSSTTAEGSSMTISTPSEGSPLLTSIPVSTTPVASPEASTLSTTPVDSNSPVITSTEVSSSPTPAEGTSMPTSTYTEGRTPLTSITVRTTPVASSAISTLSTTPVDNSTPVTTSTEARSSPTTSEGTSMPNSTPSEGTTPLTSIPVSTTPVLSSEASTLSATPIDTSTPVTTSTEATSSPTTAEGTSIPTSTLSEGMTPLTSTPVSHTLVANSEASTLSTTPVDSNSPVVTSTAVSSSPTPAEGTSIATSTPSEGSTALTSIPVSTTTVASSETNTLSTTPAVTSTPVTTYAQVSSSPTTADGSSMPTSTPREGRPPLTSIPVSTTTVASSEINTLSTTLADTRTPVTTYSQASSSPTTADGTSMPTPAYSEGSTPLTSMPLSTTLVVSSEASTLSTTPVDTSTPATTSTEGSSSPTTAGGTSIQTSTPSERTTPLAGMPVSTTLVVSSEGNTLSTTPVDSKTQVTNSTEASSSATAEGSSMTISAPSEGSPLLTSIPLSTTPVASPEASTLSTTPVDSNSPVITSTEVSSSPIPTEGTSMQTSTYSDRRTPLTSMPVSTTVVASSAISTLSTTPVDTSTPVTNSTEARSSPTTSEGTSMPTSTPSEGSTPFTSMPVSTMPVVTSEASTLSATPVDTSTPVTTSTEATSSPTTAEGTSIPTSTLSEGTTPLTSIPVSHTLVANSEVSTLSTTPVDSNTPFTTSTEASSPPPTAEGTSMPTSTSSEGNTPLTRMPVSTTMVASFETSTLSTTPADTSTPVTTYSQAGSSPTTADDTSMPTSTYSEGSTPLTSVPVSTMPVVSSEASTHSTTPVDTSTPVTTSTEASSSPTTAEGTSIPTSPPSEGTTPLASMPVSTTPVVSSEAGTLSTTPVDTSTPMTTSTEASSSPTTAEDIVVPISTASEGSTLLTSIPVSTTPVASPEASTLSTTPVDSNSPVVTSTEISSSATSAEGTSMPTSTYSEGSTPLRSMPVSTKPLASSEASTLSTTPVDTSIPVTTSTETSSSPTTAKDTSMPISTPSEVSTSLTSILVSTMPVASSEASTLSTTPVDTRTLVTTSTGTSSSPTTAEGSSMPTSTPGERSTPLTNILVSTTLLANSEASTLSTTPVDTSTPVTTSAEASSSPTTAEGTSMRISTPSDGSTPLTSILVSTLPVASSEASTVSTTAVDTSIPVTTSTEASSSPTTAEVTSMPTSTPSETSTPLTSMPVNHTPVASSEAGTLSTTPVDTSTPVTTSTKASSSPTTAEGIVVPISTASEGSTLLTSIPVSTTPVASSEASTLSTTPVDTSIPVTTSTEGSSSPTTAEGTSMPISTPSEVSTPLTSILVSTVPVAGSEASTLSTTPVDTRTPVTTSAEASSSPTTAEGTSMPISTPGERRTPLTSMSVSTMPVASSEASTLSRTPADTSTPVTTSTEASSSPTTAEGTGIPISTPSEGSTPLTSIPVSTTPVAIPEASTLSTTPVDSNSPVVTSTEVSSSPTPAEGTSMPISTYSEGSTPLTGVPVSTTPVTSSAISTLSTTPVDTSTPVTTSTEAHSSPTTSEGTSMPTSTPSEGSTPLTYMPVSTMLVVSSEDSTLSATPVDTSTPVTTSTEATSSTTAEGTSIPTSTPSEGMTPLTSVPVSNTPVASSEASILSTTPVDSNTPLTTSTEASSSPPTAEGTSMPTSTPSEGSTPLTSMPVSTTTVASSETSTLSTTPADTSTPVTTYSQASSSPPIADGTSMPTSTYSEGSTPLTNMSFSTTPVVSSEASTLSTTPVDTSTPVTTSTEASLSPTTAEGTSIPTSSPSEGTTPLASMPVSTTPVVSSEVNTLSTTPVDSNTLVTTSTEASSSPTIAEGTSLPTSTTSEGSTPLSIMPLSTTPVASSEASTLSTTPVDTSTPVTTSSPTNSSPTTAEVTSMPTSTAGEGSTPLTNMPVSTTPVASSEASTLSTTPVDSNTFVTSSSQASSSPATLQVTTMRMSTPSEGSSSLTTMLLSSTYVTSSEASTPSTPSVDRSTPVTTSTQSNSTPTPPEVITLPMSTPSEVSTPLTIMPVSTTSVTISEAGTASTLPVDTSTPVITSTQVSSSPVTPEGTTMPIWTPSEGSTPLTTMPVSTTRVTSSEGSTLSTPSVVTSTPVTTSTEAISSSATLDSTTMSVSMPMEISTLGTTILVSTTPVTRFPESSTPSIPSVYTSMSMTTASEGSSSPTTLEGTTTMPMSTTSERSTLLTTVLISPISVMSPSEASTLSTPPGDTSTPLLTSTKAGSFSIPAEVTTIRISITSERSTPLTTLLVSTTLPTSFPGASIASTPPLDTSTTFTPSTDTASTPTIPVATTISVSVITEGSTPGTTIFIPSTPVTSSTADVFPATTGAVSTPVITSTELNTPSTSSSSTTTSFSTTKEFTTPAMTTAAPLTYVTMSTAPSTPRTTSRGCTTSASTLSATSTPHTSTSVTTRPVTPSSESSRPSTITSHTIPPTFPPAHSSTPPTTSASSTTVNPEAVTTMTTRTKPSTRTTSFPTVTTTAVPTNTTIKSNPTSTPTVPRTTTCFGDGCQNTASRCKNGGTWDGLKCQCPNLYYGELCEEVVSSIDIGPPETISAQMELTVTVTSVKFTEELKNHSSQEFQEFKQTFTEQMNIVYSGIPEYVGVNITKLRHDVFQHHWHPSAKHYGDPVRP

SEQ ID NO: 7 - DNA, 3236 bp, Homo sapiens
aaagtctatacgcaataagtaagcccaaagaggcatgtttgcttggcgatgcccagcagataagccaggcaaacctcggtgtgatcgaagaagccaatttgagactcagcctagtccaggcaagctactggcacctgctgctctcaactaacctccacacaatggtgttcgcattttggaaggtctttctgatcctaagctgccttgcaggtcaggttagtgtggtgcaagtgaccatcccagacggtttcgtgaacgtgactgttggatctaatgtcactctcatctgcatctacaccaccactgtggcctcccgagaacagctttccatccagtggtctttcttccataagaaggagatggagccaatttctcacagctcgtgcctcagtactgagggtatggaggaaaaggcagtcagtcagtgtctaaaaatgacgcacgcaagagacgctcggggaagatgtagctggacctctgagatttacttttctcaaggtggacaagctgtagccatcgggcaatttaaagatcgaattacagggtccaacgatccaggtaatgcatctatcactatctcgcatatgcagccagcagacagtggaatttacatctgcgatgttaacaaccccccagactttctcggccaaaaccaaggcatcctcaacgtcagtgtgttagtgaaaccttctaagcccctttgtagcgttcaaggaagaccagaaactggccacactatttccctttcctgtctctctgcgcttggaacaccttcccctgtgtactactggcataaacttgagggaagagacatcgtgccagtgaaagaaaacttcaacccaaccaccgggattttggtcattggaaatctgacaaattttgaacaaggttattaccagtgtactgccatcaacagacttggcaatagttcctgcgaaatcgatctcacttcttcacatccagaagttggaatcattgttggggccttgattggtagcctggtaggtgccgccatcatcatctctgttgtgtgcttcgcaaggaataaggcaaaagcaaaggcaaaagaaagaaattctaagaccatcgcggaacttgagccaatgacaaagataaacccaaggggagaaagcgaagcaatgccaagagaagacgctacccaactagaagtaactctaccatcttccattcatgagactggccctgataccatccaagaaccagactatgagccaaagcctactcaggagcctgccccagagcctgccccaggatcagagcctatggcagtgcctgaccttgacatcgagctggagctggagccagaaacgcagtcggaattggagccagagccagagccagagccagagtcagagcctggggttgtagttgagcccttaagtgaagatgaaaagggagtggttaaggcataggctggtggcctaagtacagcattaatcattaaggaacccattactgccatttggaattcaaataacctaaccaacctccacctcctccttccattttgaccaaccttcttctaacaaggtgctcattcctactatgaatccagaataaacacgccaagataacagctaaatcagcaagggttcctgtattaccaatatagaatactaacaattttactaacacgtaagcataacaaatgacagggcaagtgatttctaacttagttgagttttgcaacagtacctgtgttgttatttcagaaaatattatttctctctttttaactactctttttttttattttagacagagtcttgctccgtcgcgcaggctgtgatcgtagtggtgcgatctcggctcactgcaacctccgctccctgggttcaagcgattctcctgcctgagcctcctgagtagctgggactacaggcacgtgccaccacgcccggctaattttttgtatttttagtagagatggggtttcacgttgttagccaggatggtctccatctcctgacctcatgatccgcccaccttggcctcccaaaatgctgggattacaggcatgagccactgcgcccggcctctttttagctactcttatgttccacatgcacatatgacaaggtggcattaattagattcaatattatttctaggaatagttcctcattcatttttatattgaccactaagaaaataattcatcagcattatctcatagattggaaaattttctccaaatacaatagaggagaatatgtaaagggtatacattaattggtacgtagcatttaaaatcaggtcttataattaatgcttcattcctcatattagatttcccaagaaatcaccctggtatccaatatctgagcatggcaaatttaaaaaataacacaatttcttgcctgtaaccctagcactttgggaggccgaggcaggtggatcacctgaggtcaggagttcgagaccagcctggccaacatggcgaaaccccttctctactaaaaatacaaaaattagctgggcgtggtagtgcatgcctgtaatcccagctacttgggaggctgaggcaggagaatcgcttgaacccaggaggtggaggttgcagtgagccgagattgtgccactgcactccaacctgggtgacagagtgagattccatctgaaaaacaaaaacaaaaacagaaaacaaacaaacaaaaaacaaaaaatccccacaactttgtcaaataatgtacaggcaaacactttcaaatataatttccttcagtgaatacaaaatgttgatatcataggtgatgtacaatttagttttgaatgagttattatgttatcactgtgtctgatgttatctactttgaaaggcagtccagaaaagtgttctaagtgaactcttaagatctattttagataatttcaactaattaaataacctgttttactgcctgtacattccacattaataaagcgataccaatcttatatgaatgctaatattactaaaatgcactgatatcacttcttcttcccctgttgaaaagctttctcatgatcatatttcacccacatctcaccttgaagaaacttacaggtagacttaccttttcacttgtggaattaatcatatttaaatcttactttaaggctcaataaataatactcataatgtctcattttagtgactcctaaggctagtccttttataaacaactttttctgacatagcatttatgtataataaaccagacatttaaagtgta

SEQ ID NO: 8 - protein, 423 aa, Homo sapiens
MVFAFWKVFLILSCLAGQVSVVQVTIPDGFVNVTVGSNVTLICIYTTTVASREQLSIQWSFFHKKEMEPISHSSCLSTEGMEEKAVSQCLKMTHARDARGRCSWTSEIYFSQGGQAVAIGQFKDRITGSNDPGNASITISHMQPADSGIYICDVNNPPDFLGQNQGILNVSVLVKPSKPLCSVQGRPETGHTISLSCLSALGTPSPVYYWHKLEGRDIVPVKENFNPTTGILVIGNLTNFEQGYYQCTAINRLGNSSCEIDLTSSHPEVGIIVGALIGSLVGAAIIISVVCFARNKAKAKAKERNSKTIAELEPMTKINPRGESEAMPREDATQLEVTLPSSIHETGPDTIQEPDYEPKPTQEPAPEPAPGSEPMAVPDLDIELELEPETQSELEPEPEPEPESEPGVVVEPLSEDEKGVVKA

SEQ ID NO: 9 - DNA, 3236 bp, Homo sapiens; identical to SEQ ID NO: 7

SEQ ID NO: 10 - protein, 423 aa, Homo sapiens; identical to SEQ ID NO: 8

SEQ ID NO: 11 - DNA, 2322 bp, Homo sapiens
atcattcggccctcagactgggctgggcaggtctgagagttagggaaagtccgttcccactgccctcggggagagaagaaaggagggggcaagggagaagctgctggtcggactcacaatgaaaacgctccttcttttgctgctggtgctcctggagctgggagaggcccaaggatcccttcacagggtgcccctcaggaggcatccgtccctcaagaagaagctgcgggcacggagccagctctctgagttctggaaatcccataatttggacatgatccagttcaccgagtcctgctcaatggaccagagtgccaaggaacccctcatcaactacttggatatggaatacttcggcactatctccattggctccccaccacagaacttcactgtcatcttcgacactggctcctccaacctctgggtcccctctgtgtactgcactagcccagcctgcaagacgcacagcaggttccagccttcccagtccagcacatacagccagccaggtcaatctttctccattcagtatggaaccgggagcttgtccgggatcattggagccgaccaagtctctgtggaaggactaaccgtggttggccagcagtttggagaaagtgtcacagagccaggccagacctttgtggatgcagagtttgatggaattctgggcctgggatacccctccttggctgtgggaggagtgactccagtatttgacaacatgatggctcagaacctggtggacttgccgatgttttctgtctacatgagcagtaacccagaaggtggtgcggggagcgagctgatttttggaggctacgaccactcccatttctctgggagcctgaattgggtcccagtcaccaagcaagcttactggcagattgcactggataacatccaggtgggaggcactgttatgttctgctccgagggctgccaggccattgtggacacagggacttccctcatcactggcccttccgacaagattaagcagctgcaaaacgccattggggcagcccccgtggatggagaatatgctgtggagtgtgccaaccttaacgtcatgccggatgtcaccttcaccattaacggagtcccctataccctcagcccaactgcctacaccctactggacttcgtggatggaatgcagttctgcagcagtggctttcaaggacttgacatccaccctccagctgggcccctctggatcctgggggatgtcttcattcgacagttttactcagtctttgaccgtgggaataaccgtgtgggactggccccagcagtcccctaaggaggggccttgtgtctgtgcctgcctgtctgacagaccttgaatatgttaggctggggcattctttacacctacaaaaagttattttccagagaatgtagctgtttccagggttgcaacttgaattaagaccaaacagaacatgagaatacacacacacacacacatatacacacacacacacttcacacatacacaccactcccaccaccgtcatgatggaggaattacgttatacattcatattttgtattgatttttgattatgaaaatcaaaaattttcacatttgattatgaaaatctccaaacatatgcacaagcagagatcatggtataataaatccctttgcaactccactcagccctgacaacccatccacacacggccaggcctgtttatctacactgctgcccactcctctctccagctccacatgctgtacctggatcattctgaagcaaattccgagcattacatcattttgtccataaatatttctaacatccttaaatatacaatcggaattcaagcatctcccattgtcccacaaatgtttggctgtttttgtagttggattgtttgtattaggattcaagcaaggcccatatattgcatttatttgaaatgtctgtaagtctctttccatctacagagtttagcacatttgaacgttgctggttgaaatcccgaggtgtcatttgacatggttctctgaacttatctttcctataaaatggtagttagatctggaggtctgattttgtggcaaaaatacttcctaggtggtgctgggtacttcttgttgcatcctgtcaggaggcagataatgctggtgcctctctattggtaatgttaagactgctgggtgggtttggagttcttggctttaatcattcattacaaagttcagcattttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 396 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 13 - DNA, 2228 bp, Homo sapiens
atcattcggccctcagactgggctgggcaggtctgagagttagggaaagtccgttcccactgccctcggggagagaagaaaggagggggcaagggagaagctgctggtcggactcacaatgaaaacgctccttcttttgctgctggtgctcctggagctgggagaggcccaaggatcccttcacagggtgcccctcaggaggcatccgtccctcaagaagaagctgcgggcacggagccagctctctgagttctggaaatcccataatttggacatgatccagttcaccgagtcctgctcaatggaccagagtgccaaggaacccctcatcaactacttggatatggaatacttcggcactatctccattggctccccaccacagaacttcactgtcatcttcgacactggctcctccaacctctgggtcccctctgtgtactgcactagcccagcctgcaagacgcacagcaggttccagccttcccagtccagcacatacagccagccaggtcaatctttctccattcagtatggaaccgggagcttgtccgggatcattggagccgaccaagtctctgtggaaggactaaccgtggttggccagcagtttggagaaagtgtcacagagccaggccagacctttgtggatgcagagtttgatggaattctgggcctgggatacccctccttggctgtgggaggagtgactccagtatttgacaacatgatggctcagaacctggtggacttgccgatgttttctgtctacatgagcagtaacccagaaggtggtgcggggagcgagctgatttttggaggctacgaccactcccatttctctgggagcctgaattgggtcccagtcaccaagcaagcttactggcagattgcactggataacatccaggtgggaggcactgttatgttctgctccgagggctgccaggccattgtggacacagggacttccctcatcactggcccttccgacaagattaagcagctgcaaaacgccattggggcagcccccgtggatggagaatatgctgtggagtgtgccaaccttaacgtcatgccggatgtcaccttcaccattaacggagtcccctataccctcagcccaactgcctacaccctactggacttcgtggatggaatgcagttctgcagcagtggctttcaaggacttgacatccaccctccagctgggcccctctggatcctgggggatgtcttcattcgacagttttactcagtctttgaccgtgggaataaccgtgtgggactggccccagcagtcccctaaggaggggccttgtgtctgtgcctgcctgtctgacagaccttgaatatgttaggctggggcattctttacacctacaaaaagttattttccagagaatgtagctgtttccagggttgcaacttgaattaagaccaaacagaacatgagaatacacacacacacacacatatacacacacacacacttcacacatacacaccactcccaccaccgtcatgatggaggaattacgttatacattcatattttgtattgatttttgattatgaaaatcaaaaattttcacatttgattatgaaaatctccaaacatatgcacaagcagagatcatggtataataaatccctttgcaactccactcagccctgacaacccatccacacacggccaggcctgtttatctacactgctgcccactcctctctccagctccacatgctgtacctggatcattctgaagcaaattccgagcattacatcattttgtccataaatatttctaacatccttaaatatacaatcggaattcaagcatctcccattgtcccacaaatgtttggctgtttttgtagttggattgtttgtattaggattcaagcaaggcccatatattgcatttatttgaaatgtctgtaagtctctttccatctacagagtttagcacatttgaacgttgctggttgaaatcccgaggtgtcatttgacatggttctctgaacttatctttcctataaaatggtagttagatctggaggtctgattttgtggcaaaaatacttcctaggtggtgctgggtacttcttgttgcatcctgtcaggaggcagataatgctggtgcctctctattggtaatgttaagactgctgggtgggtttggagttcttggctttaatcattcattacaaagttcagcatttta

SEQ ID NO: 14 - protein, 396 aa, Homo sapiens; identical to SEQ ID NO: 12

SEQ ID NO: 15 - DNA, 717 bp, Homo sapiens
cacggtggaagggctggggccacggggcagagaagaaaggttatctctgcttgttggacaaacagaggggagattataaaacatacccggcagtggacaccatgcattctgcaagccaccctggggtgcagctgagctagacatgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaagagaggctggttccagaggatgcatctggctcaccgggtgttccgaaaccaaagaagaaacttcgccttatcagcttcatacttcatgaaatcctgggttttcttaaccatcttttcctcattttcaatggtttaacatataatttctttaaataaaacccttaaaatctgctaaaaaaaaaaaa

SEQ ID NO: 16 - protein, 129 aa, Homo sapiens
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 17 - DNA, 737 bp, Homo sapiens
acagctgcctcttgcctcctcttcgcctccacggtggaagggctggggccacggggcagagaagaaaggttatctctgcttgttggacaaacagaggggagattataaaacatacccggcagtggacaccatgcattctgcaagccaccctggggtgcagctgagctagacatgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaagagaggctggttccagaggatgcatctggctcaccgggtgttccgaaaccaaagaagaaacttcgccttatcagcttcatacttcatgaaatcctgggttttcttaaccatcttttcctcattttcaatggtttaacatataatttctttaaataaaacccttaaaatctgctaaa

SEQ ID NO: 18 - protein, 129 aa, Homo sapiens; identical to SEQ ID NO: 16